SEQUENCE LISTING (40 sequences)

SEQ ID NO: 1 - protein, 139 aa, Plasmodium falciparum
EKKVIHGCNFSSNVSSKHTFTDSLDISLVDDSAHISCNVHLSEPKYNHLVGLNCPGDIIPDCFFQVYQPESEELEPSNIVYLDSQINIGDIEYYEDAEGDDKIKLFGIVGSIPKTTSFTCICKKDKKSAYMTVTIDSAG

SEQ ID NO: 2 - protein, 139 aa, synthetic construct
EKSVIAGCNFASNKNSKYKFAKSLDISQVDDSKHVRCEVHLSHPEYNHIVGLNCPGDIIPDCFFQVYHNSAEELEPSNIVYLEDQLNIANIEYYEDREGDDKIKMFAIIGSIPEPTSFTCICKKDKKSAYMTVFIDSSG

SEQ ID NO: 3 - protein, 139 aa, synthetic construct
EKKVIAGCNFASNKASKYKFTKSLDISLVDDSKHIRCEVHLSHPKYNHIVGLNCPGDIIPDCFFQVYQNSAEELEPSNIVYLESQLNIADIEYYEDREGDDKIKMFAIVGSIPKPTSFTCICKKDKKSAYMTVFIDSAG

SEQ ID NO: 4 - protein, 139 aa, synthetic construct
EKSVIHGCNFASNKNSKHKFAKSLDISQVDDSKHVRCEVELSHKYYNHIVGMNCPGDIIPDCFFQVYHQSSEELEPSNIVYLDDVINIDDIEYYEDREGDDKIKLFKIVGSIPKPTSFTCICKKDKKSAYMTVKIDSSG

SEQ ID NO: 5 - protein, 139 aa, synthetic construct
EKKVIHGCNFASNVASKHTFTKSLDISLVDDSAHISCNVHLSEKKYNHLVGMNCPGDIIPDCFFQVYQQESEELEPSNIVYLDSQINIGDIEYYEDAEGDDKIKLFKIVGSIPKTTSFTCICKKDKKSAYMTVTIDSAG

SEQ ID NO: 6 - protein, 139 aa, synthetic construct
EKSTIRGCNFASNKNSKWQFDNSLDISQVDDSAHIRCNVHLSYPKYNHIVGLNCPGDIIPDCFFQVYQPSSEELEPSNIVYLDSVINIDNIEYYEDREGDDKIKMFKIVGSIPKPTSFTCICKKDKKSAYMTVEIDSSG

SEQ ID NO: 7 - protein, 139 aa, synthetic construct
EKKVIAGCNFSANKASKYKFAKSLDISQVDDSKHIRCEVHLSHPKYNHIVGLNCPGDIIPDCFFQVYQNSAEELEPSNIVYLESQLNIADIEYYEDREGDDKIKMFAIVGSIPKPTSFTCICKKDKKSAYMTVFIDSAG

SEQ ID NO: 8 - protein, 139 aa, synthetic construct
EKKVIAGCNFASNVASKHTFTKSLDISLVDDSAHISCEVHLSEPKYNHLVGLNCPGDIIPDCFFQVYQNSAEELEPSNIVYLESQLNIADIEYYEDREGDDKIKMFAIVGSIPKPTSFTCICKKDKKSAYMTVFIDSAG

SEQ ID NO: 9 - protein, 139 aa, synthetic construct
EKKVIHGCNFASNVASKHTFTDSLDISLVDDSAHISCEVHLSEPKYNHLVGLNCPGDIIPDCFFQVYQNSAEELEPSNIVYLESQLNIGDIEYYEDAEGDDKIKMFAIVGSIPKPTSFTCICKKDKKSAYMTVTIDSAG

SEQ ID NO: 10 - protein, 139 aa, synthetic construct
EKKVIHGCNFASNVASKHTFTDSLDISLVDDSAHISCEVHLSEPKYNHLVGLNCPGDIIPDCFFQVYQNEAEELEPSNIVYLDSQLNIGDIEYYEDAEGDDKIKMFAIVGSIPKTTSFTCICKKDKKSAYMTVTIDSAG

SEQ ID NO: 11 - protein, 139 aa, synthetic construct
EKKVIHGCNFASNVASKHTFTDSLDISLVDDSAHISCNVHLSEPKYNHLVGLNCPGDIIPDCFFQVYQNEAEELEPSNIVYLDSQINIGDIEYYEDAEGDDKIKMFGIVGSIPKTTSFTCICKKDKKSAYMTVTIDSAG

SEQ ID NO: 12 - protein, 139 aa, synthetic construct
EKKVIHGCNFASNVASKHTFTDSLDISLVDDSAHISCNVHLSEPKYNHLVGLNCPGDIIPDCFFQVYQNEAEELEPSNIVYLDSQINIGDIEYYEDAEGDDKIKLFGIVGSIPKTTSFTCICKKDKKSAYMTVTIDSAG

SEQ ID NO: 13 - protein, 139 aa, synthetic construct
EKKVIHGCNFASNKASKHKFAKSLDISQVDDSKHIRCEVELSHKKYNHIVGMNCPGDIIPDCFFQVYQQSSEELEPSNIVYLDSVINIDDIEYYEDREGDDKIKLFKIVGSIPKPTSFTCICKKDKKSAYMTVTIDSAG

SEQ ID NO: 14 - protein, 139 aa, synthetic construct
EKKVIHGCNFASNKASKHKFTKSLDISLVDDSKHIRCEVELSHKKYNHIVGMNCPGDIIPDCFFQVYQQSSEELEPSNIVYLDSVINIDDIEYYEDREGDDKIKLFKIVGSIPKPTSFTCICKKDKKSAYMTVTIDSAG

SEQ ID NO: 15 - protein, 139 aa, synthetic construct
EKKVIHGCNFASNVASKHTFTKSLDISLVDDSAHISCEVHLSHKKYNHIVGMNCPGDIIPDCFFQVYQQSSEELEPSNIVYLDSVINIDDIEYYEDREGDDKIKLFKIVGSIPKTTSFTCICKKDKKSAYMTVTIDSAG

SEQ ID NO: 16 - protein, 139 aa, synthetic construct
EKKVIHGCNFASNVASKHTFTDSLDISLVDDSAHISCNVHLSEKKYNHLVGMNCPGDIIPDCFFQVYQQESEELEPSNIVYLDSQINIGDIEYYEDAEGDDKIKLFKIVGSIPKTTSFTCICKKDKKSAYMTVTIDSAG

SEQ ID NO: 17 - protein, 139 aa, synthetic construct
EKKVIHGCNFASNVASKHTFTDSLDISLVDDSAHISCNVHLSEPKYNHLVGMNCPGDIIPDCFFQVYQPESEELEPSNIVYLDSQINIGDIEYYEDAEGDDKIKLFKIVGSIPKTTSFTCICKKDKKSAYMTVTIDSAG

SEQ ID NO: 18 - protein, 320 aa, synthetic construct
EKSVIAGCNFASNKNSKYKFAKSLDISQVDDSKHVRCEVHLSHPEYNHIVGLNCPGDIIPDCFFQVYHNSAEELEPSNIVYLEDQLNIANIEYYEDREGDDKIKMFAIIGSIPEPTSFTCICKKDKKSAYMTVFIDSSGGTGSGGGGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 19 - protein, 320 aa, synthetic construct
EKKVIAGCNFASNKASKYKFTKSLDISLVDDSKHIRCEVHLSHPKYNHIVGLNCPGDIIPDCFFQVYQNSAEELEPSNIVYLESQLNIADIEYYEDREGDDKIKMFAIVGSIPKPTSFTCICKKDKKSAYMTVFIDSAGGTGSGGGGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 20 - protein, 320 aa, synthetic construct
EKSVIHGCNFASNKNSKHKFAKSLDISQVDDSKHVRCEVELSHKYYNHIVGMNCPGDIIPDCFFQVYHQSSEELEPSNIVYLDDVINIDDIEYYEDREGDDKIKLFKIVGSIPKPTSFTCICKKDKKSAYMTVKIDSSGGTGSGGGGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 21 - protein, 320 aa, synthetic construct
EKKVIHGCNFASNVASKHTFTKSLDISLVDDSAHISCNVHLSEKKYNHLVGMNCPGDIIPDCFFQVYQQESEELEPSNIVYLDSQINIGDIEYYEDAEGDDKIKLFKIVGSIPKTTSFTCICKKDKKSAYMTVTIDSAGGTGSGGGGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 22 - protein, 167 aa, synthetic construct
MLSKDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 23 - protein, 163 aa, synthetic construct
DIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 24 - protein, 173 aa, synthetic construct
ESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 25 - protein, 8 aa, synthetic construct
GTGSGGGG

SEQ ID NO: 26 - protein, 4 aa, synthetic construct
GSGG

SEQ ID NO: 27 - protein, 448 aa, Plasmodium falciparum
MMLYISAKKAQVAFILYIVLVLRIISGNNDFCKPSSLNSEISGFIGYKCNFSNEGVHNLKPDMRERRSIFCTIHSYFIYDKIRLIIPKKSSSPEFKILPEKCFQKVYTDYENRVETDISELGLIEYEIEENDTNPNYNERTITISPFSPKDIEFFCFCDNTEKVISSIEGRSAMVHVRVLKYPHNILFTNLTNDLFTYLPKTYNESNFVSNVLEVELNDGELFVLACELINKKCFQEGKEKALYKSNKIIYHKNLTIFKAPFYVTSKDVNTECTCKFKNNNYKIVLKPKYEKKVIHGCNFSSNVSSKHTFTDSLDISLVDDSAHISCNVHLSEPKYNHLVGLNCPGDIIPDCFFQVYQPESEELEPSNIVYLDSQINIGDIEYYEDAEGDDKIKLFGIVGSIPKTTSFTCICKKDKKSAYMTVTIDSAYYGFLAKTFIFLIVAILLYI

SEQ ID NO: 28 - protein, 7 aa, synthetic construct
ALWXFGS

SEQ ID NO: 29 - protein, 6 aa, synthetic construct
ALWYFG

SEQ ID NO: 30 - protein, 8 aa, synthetic construct
ALWAYFGS

SEQ ID NO: 31 - protein, 400 aa, Plasmodium falciparum
MMRKLAILSVSSFLFVEALFQEYQCYGSSSNTRVLNELNYDNAGTNLYNELEMNYYGKQENWYSLKKNSRSLGENDDGNNNNGDNGREGKDEDKRDGNNEDNEKLRKPKHKKLKQPADGNPDPNANPNVDPNANPNVDPNANPNVDPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVFNVVNSSIGLIMVLSFLFLN

SEQ ID NO: 32 - protein, 108 aa, synthetic construct
NPDPNANPNVDPNANPNANPNANPNANPNANPNAEPSDKHIKEYLNKIQNSLSTEWSPCSVTCGNGIQVRIKPGSANKPKDELDYANDIEKKICKMEKCSSVFNVVNS

SEQ ID NO: 33 - protein, 250 aa, synthetic construct
NPDPNANPNVDPNANPNANPNANPNANPNANPNAEPSDKHIKEYLNKIQNSLSTEWSPCSVTCGNGIQVRIKPGSANKPKDELDYANDIEKKICKMEKCSSVFNVVNSGGSEKKVIHGCNFASNVASKHTFTKSLDISLVDDSAHISCNVHLSEKKYNHLVGMNCPGDIIPDCFFQVYQQESEELEPSNIVYLDSQINIGDIEYYEDAEGDDKIKLFKIVGSIPKTTSFTCICKKDKKSAYMTVTIDSAG

SEQ ID NO: 34 - protein, 247 aa, synthetic construct
EKKVIHGCNFASNVASKHTFTKSLDISLVDDSAHISCNVHLSEKKYNHLVGMNCPGDIIPDCFFQVYQQESEELEPSNIVYLDSQINIGDIEYYEDAEGDDKIKLFKIVGSIPKTTSFTCICKKDKKSAYMTVTIDSAGNPDPNANPNVDPNANPNANPNANPNANPNANPNAEPSDKHIKEYLNKIQNSLSTEWSPCSVTCGNGIQVRIKPGSANKPKDELDYANDIEKKICKMEKCSSVFNVVNS

SEQ ID NO: 35 - protein, 433 aa, synthetic construct
NPDPNANPNVDPNANPNANPNANPNANPNANPNAEPSDKHIKEYLNKIQNSLSTEWSPCSVTCGNGIQVRIKPGSANKPKDELDYANDIEKKICKMEKCSSVFNVVNSGGSEKKVIHGCNFASNVASKHTFTKSLDISLVDDSAHISCNVHLSEKKYNHLVGMNCPGDIIPDCFFQVYQQESEELEPSNIVYLDSQINIGDIEYYEDAEGDDKIKLFKIVGSIPKTTSFTCICKKDKKSAYMTVTIDSAGGTGSGGGGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 36 - protein, 430 aa, synthetic construct
EKKVIHGCNFASNVASKHTFTKSLDISLVDDSAHISCNVHLSEKKYNHLVGMNCPGDIIPDCFFQVYQQESEELEPSNIVYLDSQINIGDIEYYEDAEGDDKIKLFKIVGSIPKTTSFTCICKKDKKSAYMTVTIDSAGNPDPNANPNVDPNANPNANPNANPNANPNANPNAEPSDKHIKEYLNKIQNSLSTEWSPCSVTCGNGIQVRIKPGSANKPKDELDYANDIEKKICKMEKCSSVFNVVNSGTGSGGGGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 37 - protein, 12 aa, Plasmodium falciparum
NPDPNANPNVDP

SEQ ID NO: 38 - protein, 74 aa, Plasmodium falciparum
EPSDKHIKEYLNKIQNSLSTEWSPCSVTCGNGIQVRIKPGSANKPKDELDYANDIEKKICKMEKCSSVFNVVNS

SEQ ID NO: 39 - protein, 4 aa, Plasmodium falciparum
NANP

SEQ ID NO: 40 - protein, 175 aa, synthetic construct
ESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

(19) United States
(12) Patent Application Publication
(10) Pub. No.: US 2026/0207725 A1
(43) Pub. Date: Jul. 23, 2026

(54) STABILIZED PFS48/45 PROTEINS AND USES THEREOF

(71) Applicant: The United States of America, as represented by the Secretary, Department of Health and Human, Bethesda, MD (US)

(72) Inventors: Niraj Harish TOLIA, Bethesda, MD (US); Thayne Henderson DICKEY, Bethesda, MD (US); Richi GUPTA, Bethesda, MD (US); Dashuang SHI, Bethesda, MD (US)

(21) Appl. No.: 19/142,034

(22) PCT Filed: Dec. 22, 2023
(86) PCT No.: PCT/US2023/085849

§ 371 Date: Jun. 20, 2025

Related U.S. Application Data
(60) Provisional application No. 63/476,897, filed on Dec. 22, 2022.

(51) Int. Cl. A61K 39/015; A61P 37/04; C07K 14/00

(52) U.S. Cl. CPC A61K 39/015; A61P 37/04; C07K 14/00

(57) ABSTRACT

Disclosed herein are modified proteins that may be used to protect an individual against, or treat an individual for, organisms that cause malaria. Specifically, the modified proteins comprise a modified 6C domain from Plasmodium spp P48/45 protein. Such modified proteins retain an epitope that elicits neutralizing antibodies against the P48/45 protein. Also disclosed are nucleic acid molecules encoding the modified proteins, nanoparticles displaying the modified protein, as well as methods of using the modified proteins and nanoparticles.

### CROSS-REFERENCE TO RELATED APPLICATIONS

This application claims the benefit of priority of U.S. Provisional Patent Application No. 63/476,897, filed Dec. 22, 2022, which is incorporated herein by reference in its entirety.

### STATEMENT REGARDING FEDERALLY SPONSORED RESEARCH & DEVELOPMENT

This invention was made with U.S. government support from the National Institutes of Health. The U.S. government may have certain rights in the disclosed invention.

### SEQUENCE LISTING

This application contains a sequence listing in paper format and in computer readable format, the contents of which are hereby incorporated by reference.

### BACKGROUND OF THE INVENTION

Vaccines that disrupt the Plasmodium parasite lifecycle would be powerful tools in combatting malaria. Progress towards malaria control has largely stalled in the last decade and has reversed in some endemic areas highlighting the need for durable interventions that include vaccines. The recent approval of the partially effective RTS,S vaccine demonstrates both the utility of a vaccine in malaria control as well as the need for additional improvements in malaria vaccine development. One strategy for improving malaria vaccines is to target additional stages of the parasite life cycle such as transmission from human to mosquito.

The Pfs48/45 protein is a promising malaria transmission-blocking vaccine (TBV) candidate. Pfs48/45 is present on the surface of sexual stage parasites transferred from human to mosquito during a blood meal and in the mosquito prior to fertilization, with peak surface expression on gametes and zygotes. Pfs48/45 is required for efficient parasite fertilization and oocyst formation in the mosquito, and serum from animals immunized with Pfs48/45 blocks oocyst development. Human infection elicits antibodies to Pfs48/45, suggesting vaccine-induced immunity could be boosted by natural infection. The precise function of Pfs48/45 remains unknown, although an interaction with another TBV candidate, Pfs230, has been proposed.

Pfs48/45 contains three 6-cys domains (D1-3) and a C-terminal GPI anchor (FIG. 1A). Functions for each 6-cys domain have yet to be assigned, including the putative interaction site with Pfs230, limiting the ability to target biologically critical surfaces. Instead, monoclonal antibodies have been isolated, and their binding has been mapped on the protein. Antibody mapping studies found that antibodies to the N-terminal domain (D1) have poor transmission-reducing activity (TRA), antibodies to D2 have limited TRA, and antibodies to D3 can have potent TRA. The high-resolution crystal structures of a potently neutralizing antibody, 85RF45.1, and its humanized counterpart, TB31F, were solved in complex with D3, defining a potently neutralizing epitope. This epitope contains only rare polymorphisms that do not substantially disrupt mAb binding, suggesting that a vaccine that elicits antibodies to this epitope could be broadly neutralizing.

The crystal structure of D3 also sheds light on stability and production challenges for Pfs48/45 that have limited vaccine development. The crystallized Pfs48/45 protein was produced in a eukaryotic expression system and expression required preservation of an NxS/T amino acid motif recognized by the oligosaccharyltransferase complex in these systems. Indeed, an N-acetyl-glucosamine (NAG) was observed at position 303 in a loop of the protein, and NAG303 formed visible stabilizing contacts with a neighboring loop. There is no evidence that native Pfs48/45 is glycosylated and N-linked glycosylation in malaria proteins is generally limited, meaning that NAG303 may be a non-native mechanism of antigen stabilization. Consistent with this hypothesis, NAG303 lies at a surface predicted to be proximal to the N-terminal D2, which was not included in the crystallization construct. Together, these findings suggest that D3 requires stabilization for production, which can be achieved by glycosylation. However, the large glycans appended by eukaryotic systems other than Plasmodium do not reflect the native protein and may obscure neutralizing epitopes on the protein.

Stabilizer for Protein Expression and Epitope Design (SPEEDesign) is a ROSETTA-based computational and in vitro screening pipeline that designs antigens resulting in potent and durable vaccines. The objectives of SPEEDesign are to improve the protective efficacy of a given antigen by: 1) focusing the immune response towards potently neutralizing antibody epitopes on the antigen; 2) reducing or eliminating immune responses to poorly neutralizing and/or immunodominant epitopes within the antigen; 3) optimizing the thermal stability of the antigen to increase its durability in vivo following immunization; 4) promoting conformational states of a protein antigen that may be hidden, for example by removing the glycan on pfs48/45 D3 that may mask neutralizing epitopes. SPEEDesign has proven successful in the development of improved SARS-COV-2 antigens. Details of the SPEEDesign method are disclosed in WO2022/178545, which is incorporated herein by reference in its entirety.

Non-glycosylated Pfs48/45 D3 can be produced in bacterial systems, but the protein requires alternative means of stabilization and careful optimization to achieve proper disulfide bond formation. The protein can be expressed in E. coli, but requires fusion to maltose binding protein and co-expression of four chaperones. The protein can also be produced in L. lactis but requires fusion to Pfs230 or GLURP to produce high yields of properly folded protein. The GLURP-fusion protein (R0.6C) is a promising pre-clinical vaccine candidate and several years of work have led to a reproducible purification process, however, the potential of a Pfs48/45 D3 vaccine has not been completely evaluated because the domain cannot be produced in isolation. Here is disclosed a method for creation of a stand-alone Pfs48/45 D3 antigen suitable for TBV development. A SPEEDesign computational design process was used to create immunogens suitable for expression in a eukaryotic system without requiring glycosylation. These immunogens can be expressed at yields much greater than the glycosylated wildtype (WT) protein; they have increased thermostability, and they retain the potent transmission-blocking epitope. Immunization of rats with these immunogens also elicited transmission blocking antibodies. Further optimization of the immunogens resulted in a clinically relevant nanoparticle vaccine that elicits potent TRA in rats with two low-dose immunizations.

### SUMMARY OF THE DISCLOSURE

One aspect of the disclosure is a modified 6C domain from a 48/45 protein (P48/45) of a Plasmodium spp., wherein the amino acid sequence of the modified 6C domain has at least about 70% homology over the entire length of the sequence of a wild-type (wt) P48/45 6C domain, wherein the amino acid sequence of the modified 6C domain comprises one or more mutations, relative to the wt 6C domain amino acid sequence, that increase the stability of the modified 6C domain relative to the stability of the wt 6C domain, wherein the modified 6C domain retains a 85RF45.1 epitope such that Fab85RF45.1 binds the modified 6C domain with high affinity, and wherein the one or more mutations are at an amino acid position selected from the group consisting of an amino acid position corresponding to S11 of SEQ ID NO:1, an amino acid position corresponding to S15 of SEQ ID NO:1, an amino acid position corresponding to D22 of SEQ ID NO:1, and an amino acid position corresponding to P69 of SEQ ID NO:1. The modified 6C domain of claim 1, wherein the modified 6C domain is derived from a wt 6C domain from a Plasmodium (P.) species that causes malaria. In certain aspects, the modified 6C domain is derived from a wt 6C domain from a Plasmodium species selected from the group consisting of P. falciparum (Pfs), P. vivax (Pvx), P. malariae (Pml), and P. ovale (Pov). The wt 6C domain comprises an amino acid sequence at least 85% identical, at least 90% identical, at least 95% identical, at last 97% identical, or 100% identical, to SEQ ID NO:1. In certain aspects, the modified 6C domain may comprise a Tₘ of at least, at least 52 C, at least 54 C, at least 56 C, at least 58 C, at least 60° C., at least 65° C., or at least 70° C. In certain aspects, the contact residues of the 85RF45.1 epitope in the modified 6C domain may comprise at least 10 amino, optionally at last 15, optionally at least 20, optionally at least 24 acid residues selected from the group consisting of D31, P55, G56, D57, 158, 159, P60, D61, F64, Q65, L74, E75, P76, S77, 179, Y81, E95, D100, K102, K104, 1121, K123, K124, D125, K126, and S128 of SEQ ID NO:1. The contact residues of the 85RF45.1 epitope in the modified 6C domain may comprise at least 10, optionally at least 15, optionally at least 20, optionally at least 24, or all, amino acid residues selected from the group consisting of: an aspartic acid at a position corresponding to position 31 of SEQ ID NO:1 [D321], a proline at a position corresponding to position 55 of SEQ ID NO:1 [P345], a glycine at a position corresponding to position 56 of SEQ ID NO:1 [G346], an aspartic acid at a position corresponding to position 57 of SEQ ID NO:1 [D347], an isoleucine at a position corresponding to position 58 of SEQ ID NO:1 [1348], a isoleucine at a position corresponding to position 59 of SEQ ID NO:1 [1349], a proline at a position corresponding to position 60 of SEQ ID NO:1 [P350], an aspartic acid at a position corresponding to position 61 of SEQ ID NO:1 [D351], a phenylalanine at a position corresponding to position 64 of SEQ ID NO:1 [F354], a glutamine at a position corresponding to position 65 of SEQ ID NO:1 [Q355], a leucine at a position corresponding to position 74 of SEQ ID NO:1 [L364], a glutamic acid at a position corresponding to position 75 of SEQ ID NO:1 [E365], a proline at a position corresponding to position 76 of SEQ ID NO:1 [P366], a serine at a position corresponding to position 77 of SEQ ID NO:1 [P367], an isoleucine at a position corresponding to position 79 of SEQ ID NO:1 [1369], a tyrosine at apposition corresponding to position 81 of SEQ ID NO: 1 [Y371], a glutamic acid at a position corresponding to position 95 of SEQ ID NO:1 [E385], an aspartic acid a position corresponding to position 100 of SEQ ID NO:1 [D390], a lysine at a position corroding to position 102 of SEQ ID NO:1 [K392], a lysine at a position corresponding to position 104 of SEQ ID NO:1 [K394], an isoleucine at a position corresponding to position 121 of SEQ ID NO:1 [1411], a lysine at a position corresponding to position 123 of SEQ ID NO: 1 [K413], a lysine at a position corresponding to position 124 of SEQ ID NO:1 [K414], an aspartic acid at a position corresponding to position 125 of SEQ ID NO:1 [D415], a lysine at a position corresponding to position 126 of SEQ ID NO:1 [K416], and, a serine at a position corresponding to position 128 of SEQ ID NO:1 [S418]. In certain aspects, the one or more mutations are made at one or more positions selected from the group consisting of: an amino acid position corresponding to position 3,6, 11, 14, 15, 18, 19, 21, 22, 28, 33, 35, 36, 38, 40, 43, 44, 45, 49, 52, 68, 69, 70, 71, 83, 84, 85, 86, 89, 90, 97, 105, 107, 109, 114, 115, 134, adn134, of SEQ ID NO:1. In certain aspects, the modified 6C domain comprise a mutation or sequence disclosed herein.

One aspect of the disclosure is an isolated protein comprising a modified 6C domain of the disclosure. In certain aspect, the isolated protein comprises a full-length Plasmodium 48/45 protein in which the 6C domain comprise a modified 6C domain of the disclosure.

One aspect of the disclosure is an isolated protein comprising an amino acid sequence having at least about 70% homology over the entire length of SEQ IID NO: 1, wherein the amino acid sequence comprises one or more mutations, relative to SEQ ID NO:1 that increase the stability of the acid relative to the stability of a protein comprising SEQ ID NO:1, wherein the one or more mutations are at one or more positions selected form the group consisting of an amino acid position corresponding to S11 of SEQ ID NO:1, an amino acid position corresponding to S15 of SEQ ID NO:1, an amino acid position corresponding to D22 of SEQ ID NO:1, and an amino acid position corresponding to P69 of SEQ ID NO:1. In certain aspects, the amino acid sequence may comprise at least 10 amino, optionally at least 15, optionally at least 20, optionally at least 24 acid residues selected from the group consisting of: an aspartic acid at a position corresponding to position 31 of SEQ ID NO:1 [D321], a proline at a position corresponding to position 55 of SEQ ID NO:1 [P345], a glycine at a position corresponding to position 56 of SEQ ID NO:1 [G346], an aspartic acid at a position corresponding to position 57 of SEQ ID NO:1 [D347], a isoleucine at a position corresponding to position 58 of SEQ ID NO:1 [1348], an isoleucine at a position corresponding to position 59 of SEQ ID NO:1 [1349], a proline at a position corresponding to position 60 of SEQ ID NO:1 [P350], an aspartic acid at a position corresponding to position 61 of SEQ ID NO:1 [D351], a phenylalanine at a position corresponding to position 64 of SEQ ID NO:1 [F354], a glutamine at a position corresponding to position 65 of SEQ ID NO:1 [Q355], a leucine at a position corresponding to position 74 of SEQ ID NO:1 [L364], a glutamic acid at a position corresponding to position 75 of SEQ ID NO:1 [E365], a proline at a position corresponding to position 76 of SEQ ID NO:1 [P366], a serine at a position corresponding to position 77 of SEQ ID NO:1 [P367], an isoleucine at a position corresponding to position 79 of SEQ ID NO:1 [1369], a tyrosine at apposition corresponding to position 81 of SEQ ID NO: 1 [Y371], a glutamic acid at a position corresponding to position 95 of SEQ ID NO:1 [E385], an aspartic acid a position corresponding to position 100 of SEQ ID NO:1 [D390], a lysine at a position corroding to position 102 of SEQ ID NO:1 [K392], a lysine at a position corresponding to position 104 of SEQ ID NO:1 [K394], an isoleucine at a position corresponding to position 121 of SEQ ID NO:1 [1411], a lysine at a position corresponding to position 123 of SEQ ID NO: 1 [K413], a lysine at a position corresponding to position 124 of SEQ ID NO:1 [K414], an aspartic acid at a position corresponding to position 125 of SEQ ID NO:1 [D415], a lysine at a position corresponding to position 126 of SEQ ID NO:1 [K416], and, a serine at a position corresponding to position 128 of SEQ ID NO:1 [S418]. In certain aspects, the one or more mutations are made at one or more positions selected from the group consisting of: 3,6, 11, 14, 15, 18, 19, 21, 22, 28, 33, 35, 36, 38, 40, 43, 44, 45, 49, 52, 68, 69, 70, 71, 83, 84, 85, 86, 89, 90, 97, 105, 107, 109, 114, 115, 134, adn134, of SEQ ID NO:1. In certain aspects, the amino acid sequence may lack an N-glycosylation signal sequence. In certain aspects, the isolated protein may comprise at least about 80%, at least about 85%, at least about 90%, at least about 92%, at least about 94%, at least about 96%, at least about 98%, or at least about 99%, or 100%, homology, or identity, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO: 12, SEQ ID NO:13, SEQ ID NO:14, SEQ ID NO:15, SEQ ID NO:16, and SEQ ID NO: 17. In certain aspects, the amino acid sequence may comprise a mutation or sequence disclosed herein.

One aspect of the disclosure is a fusion protein comprising a monomeric, self-assembling subunit protein joined to a modified 6C domain of the disclosure, an isolated protein of the disclosure, an immunogen of the disclosure, a modified CSP of the disclosure, or a fusion protein of the disclosure. In certain aspects, the monomeric, self-assembling subunit protein may ferritin, dihydrolipoyl acetyltransferase (E2P), Lumazine Synthase (LuS), hepatitis B surface antigen (HBsAg), or human papilloma virus protein LI (HPV LI). In certain aspects, the fusion protein may comprise at least 25, at least 50, or at least 10 contiguous amino acid residues from ferritin, dihydrolipoyl acetyltransferase (E2P), Lumazine Synthase (LuS), hepatitis B surface antigen (HBsAg), or human papilloma virus protein LI (HPV LI). In certain aspects, the monomeric, self-assembling subunit protein may comprise an amino acid sequence having at least 90%, at least 85%, at least 90%, at least 95%, at least 97%, or 100%, homology or identity, with ferritin, dihydrolipoyl acetyltransferase (E2P), Lumazine Synthase (LuS), hepatitis B surface antigen (HBsAg), or human papilloma virus protein LI (HPV LI). In certain aspects, the self-assembling monomeric subunit is joined directly to the modified 6C domain or the isolated protein of the disclosure, or it may be joined through a linker.

One aspect of the disclosure is a nucleic acid molecule encoding a modified 6C domain of the disclosure, an isolated protein of the disclosure, an immunogen of the disclosure, a modified CSP of the disclosure, or a fusion protein of the disclosure. In certain aspects, the nucleic acid molecule may be operably linked to a promoter

One aspect of the disclosure is an expression vector comprising a nucleic acid molecule of the disclosure. The expression vector may be a plasmid or a viral vector.

One aspect of the disclosure is a cell comprising a nucleic acid molecule or an expression vector of the disclosure.

One aspect of the disclosure is a nanoparticle comprising a modified 6C domain of the disclosure, an isolated protein of the disclosure, an immunogen of the disclosure, a modified CSP of the disclosure, or a fusion protein of the disclosure.

One aspect of the disclosure is a composition comprising a modified 6C domain of the disclosure, an isolated protein of the disclosure, an immunogen of the disclosure, a modified CSP of the disclosure, a fusion protein of the disclosure, a nucleic acid molecule of the disclosure, an expression vector of the disclosure, a cell of the disclosure, or a nanoparticle of the disclosure. The composition may comprise a buffer, a pharmaceutically acceptable excipient, a diluent, a preservative, a stabilizer, an immune stimulant, or an adjuvant.

One aspect of the disclosure is a vaccine composition comprising a modified 6C domain of the disclosure, an isolated protein of the disclosure, an immunogen of the disclosure, a modified CSP of the disclosure, a fusion protein of the disclosure, a nucleic acid molecule of the disclosure, an expression vector of the disclosure, a cell of the disclosure, a nanoparticle of the disclosure, or a composition of the disclosure.

One aspect of the disclosure is a method to elicit an immune response against a Plasmodium spp. in an individual, comprising administering to the individual a modified 6C domain of the disclosure, an isolated protein of the disclosure, an immunogen of the disclosure, a modified CSP of the disclosure, a fusion protein of the disclosure, a nucleic acid molecule of the disclosure, an expression vector of the disclosure, a cell of the disclosure, a nanoparticle of the disclosure, or a vaccine composition of the disclosure.

One aspect of the disclosure is a method to protect an individual against malaria, comprising administering to the individual a modified 6C domain of the disclosure, an isolated protein of the disclosure, an immunogen of the disclosure, a modified CSP of the disclosure, a fusion protein of the disclosure, a nucleic acid molecule of the disclosure, an expression vector of the disclosure, a cell of the disclosure, a nanoparticle of the disclosure, or a vaccine composition of the disclosure.

One aspect of the disclosure is a method to vaccinate an individual against malaria, comprising administering to the individual a modified 6C domain of the disclosure, an isolated protein of the disclosure, an immunogen of the disclosure, a modified CSP of the disclosure, a fusion protein of the disclosure, a nucleic acid molecule of the disclosure, an expression vector of the disclosure, a cell of the disclosure, a nanoparticle of the disclosure, or a vaccine composition of the disclosure.

One aspect of the disclosure is use of a modified 6C domain of the disclosure, an isolated protein of the disclosure, a fusion protein of the disclosure, a nucleic acid molecule of the disclosure, an expression vector of the disclosure, a cell of the disclosure, a nanoparticle of the disclosure, or a vaccine composition of the disclosure, for the preventing infection of an individual by Plasmodium.

One aspect of the disclosure is use of a modified 6C domain of the disclosure, an isolated protein of the disclosure, an immunogen of the disclosure, a modified CSP of the disclosure, a fusion protein of the disclosure, a nucleic acid molecule of the disclosure, an expression vector of the disclosure, a cell of the disclosure, a nanoparticle of the disclosure, or a vaccine composition of the disclosure, for the treatment of malaria.

One aspect of the disclosure is a method to detect anti-P48/45 antibodies in a sample, comprising: contacting the sample with a modified 6C domain of the disclosure, an isolated protein of the disclosure, a fusion protein of the disclosure, a nanoparticle of the disclosure, or a composition of the disclosure; and detecting the complex, if any, thereby detecting the presence of the anti-P48/45 antibody.

One aspect of the disclosure is a method of producing a modified 6C domain of the disclosure, comprising incubating the nucleic acid molecule of the disclosure under conditions suitable for expression of an encoded protein from the nucleic acid molecule, wherein the nucleic acid molecule encodes the modified 6C domain.

One aspect of the disclosure is a method of producing isolated protein of the disclosure, comprising incubating a nucleic acid molecule of the disclosure under conditions suitable for expression of an encoded protein from the nucleic acid molecule, wherein the nucleic acid molecule encodes the isolated protein.

One aspect of the disclosure is a method of producing a fusion protein of the disclosure, comprising incubating a nucleic acid molecule of the disclosure under conditions suitable for expression of an encoded protein from the nucleic acid molecule, wherein the nucleic acid molecule encodes the fusion protein.

One aspect of the disclosure is a method of producing a nanoparticle of the disclosure, comprising a) incubating a cell comprising a nucleic acid molecule encoding a fusion protein of the disclosure, wherein the incubation conditions are suitable for expression of the encoded fusion protein; and b) recovering the nanoparticle.

One aspect of the disclosure is a kit comprising a modified 6C domain of the disclosure, an isolated protein of the disclosure, an immunogen of the disclosure, a modified CSP of the disclosure, a fusion protein of the disclosure, a nucleic acid molecule of the disclosure, an expression vector of the disclosure, a cell of the disclosure, a nanoparticle of the disclosure, a composition of the disclosure, or a vaccine composition of the disclosure.

### BRIEF DESCRIPTION OF THE DRAWINGS

FIGS. 1A-1F illustrate the design of a Pfs48/45 D3 antigen with improved yield and thermostability. FIG. 1A shows an overview of Pfs48/45 domain architecture. FIG. 1B shows the crystal structure of Pfs48/45 D3 (grey) in complex with the neutralizing mAb 85RF45.1 (blue) (PDB ID: 6E62). The predicted interface with D2 (red) was heavily designed while the neutralizing epitope (cyan) was retained. The N-glycan is shown in pink. FIG. 1C shows the reactivity of cell-free supernatant with neutralizing mAb TB31F for each transfected candidate. FIG. 1D shows size-exclusion chromatography profile of lead immunogens and WT Pfs48/45 after nickel purification.

FIG. 1E-SDS-PAGE analysis of purified lead immunogens. FIG. 1F shows a summary of melting temperature (Tₘ) and purification yields for lead immunogens and WT Pfs48/45.

FIG. 2 shows size-exclusion chromatogram of nickel purified protein illustrating that monomeric WT D3 cannot be purified after mutation of the NxS/T motif.

FIG. 3 shows representative DSF traces of lead immunogens and

### WT Pfs48/45.

FIGS. 4A-D show representative BLI traces of TB31F mAb binding to WT D3 and lead immunogens. Top curves contain 30 nM antigen with a 2-fold dilution series to 0.469 nM.

FIGS. 5A-5C show immunization of rats with Pfs48/45 immunogens elicits transmission reducing activity (TRA). FIG. 5A illustrates the immunization and blood draw regimen. FIG. 5B shows IgG antibody titers to WT Pfs48/45 D3. Median values and 95% confidence intervals are displayed. FIG. 5C shows TRA of serum from immunized rats with median values displayed. Dashed line indicates 0% TRA relative to adjuvant only serum.

FIG. 6 shows that immunogens 15 and 17 elicit TRA in pooled serum from immunized rats. Dashed line indicates 0% TRA relative to adjuvant only serum.

FIGS. 7A-7H show creation of nanoparticles displaying optimized immunogens. FIG. 7A illustrates the antigen optimization process first reverting designed amino acids (red) back to WT identity (grey), followed by display on a 24-copy ferritin nanoparticle (blue). FIG. 7B shows screening of optimized immunogen monomers. Nickel purification yields are reported for all constructs. Tₘ was measured for those constructs with sufficient yields. Immunogens 15-2 and 17-4 were identified as optimized leads for nanoparticle display. FIGS. 7C & 7D shows size exclusion chromatogram following Ni purification of immunogens derived from the parent immunogen 15 (FIG. 7C) and immunogen 17 (FIG. 17D). FIGS. 7E & 7F show differential scanning fluorimetry of the high-yield optimized immunogens based on parent immunogen 15 (FIG. 7E) and immunogen 17 (FIG. 7F). Final immunogens (15-2 and 17-4) are shown in bold. 7G shows SDS-PAGE analysis of purified immunogens fused with ferritin. 7H shows size-exclusion chromatogram of purified particles after freeze thaw, indicating formation of a stable particle. FIG. 7I shows negative-stain EM micrographs of purified nanoparticles displaying optimized immunogens. The 2D class averages are shown below each micrograph.

FIG. 8 shows sequence alignment of designed immunogens and an independently derived stabilized immunogen 6C, mAgE2.

FIGS. 9A-9C show two low-dose immunizations with a Pfs48/45 D3 immunogen nanoparticle elicits high TRA. FIG. 9A illustrates immunization and blood draw schedule for Wistar rats. FIG. 9B shows IgG antibody titers to WT Pfs48/45 D3. Medians and 95% confidence intervals are displayed. Dashed line indicates the limit of detection. P-values displayed were calculated using a Kruskal-Wallis test followed by a Dunn's comparison to adjuvant only, corrected for multiple comparisons. FIG. 9C shows TRA of serum from immunized rats with median values indicated. Dashed line indicates 0% TRA relative to adjuvant only serum. P-value displayed was calculated using a two-tailed Mann-Whitney test.

FIG. 10 shows that Immunogen 17-4-ferritin elicits high TRA in rats. SMFAs were performed after pooling serum from individual rats. Dashed line indicates 0% TRA relative to adjuvant only serum.

FIG. 11 shows that the quality of antibodies improves through the antigen design process. Antibody quality is quantified by normalizing % TRA to the levels of anti-Pfs48/45 IgG in each animal. Animals with negative % TRA values are plotted at the lowest normalized value (dashed line).

FIG. 12 shows an alignment of the amino acid sequence of wt P48/45 6C domain with the modified 6C domains of the disclosure. The modified amino acid residues in each immunogen are highlighted.

FIG. 13 shows the mutations introduced into the wt amino acid sequence of wt P48/45 6C domain to produce each of the designated immunogens.

FIG. 14 shows an exemplary sequence of the circumsporozoite protein (CSP) of Plasmodium falciparum. The highlighted region is the leader sequence. The underlined region is the junction region. The bolded amino acids indicate which amino acids may be joined together to produce an exemplary modified CSP of the disclosure.

FIG. 15 Shows antibody titers resulting from immunization of mice with a 17-4-CSP fusion protein, a CSP-17-4 fusion protein, nanoparticles produced from fusion proteins comprising either CSP (CSP-17-4-ferritin) or 17-4 (17-4-CSP-ferritin) at the N-terminus, nanoparticles displaying 17-4 or nanoparticles displaying CSP.

FIG. 16 the percent of immunized mice surviving at day 64 following challenge with 1,000 PbPfCSP sporozoites.

FIGS. 17A & 17B show titer of anti-CSP antibodies in rabbits at various times following immunization with a 17-4-CSP fusion protein, a CSP-17-4 fusion protein, nanoparticles produced from fusion proteins comprising either CSP (CSP-17-4-ferritin) or 17-4 (17-4-CSP-ferritin) at the N-terminus, nanoparticles displaying 17-4 or nanoparticles displaying CSP.

FIGS. 18A & 18B show titer of anti-Pfs48/45 antibodies in rabbits at various times following immunization with a 17-4-CSP fusion protein, a CSP-17-4 fusion protein, nanoparticles produced from fusion proteins comprising either CSP (CSP-17-4-ferritin) or 17-4 (17-4-CSP-ferritin) at the N-terminus, nanoparticles displaying 17-4 or nanoparticles displaying CSP.

### DETAILED DESCRIPTION OF THE INVENTION

The disclosure relates to a novel anti-malarial vaccine. More specifically, disclosed herein are novel protein sequences that can be used to protect against, or treat, malaria in an individual. The disclosed sequences are not found in nature but are derived from the amino acid sequence of the Plasmodium falciparum (Pfs) Pfs48/45 protein, and in particular, the third domain (aka., PFS48/45 6C or the 6C domain) of Pfs48/45. Pfs48/45 6C has been established as candidate transmission blocking vaccine. However, due to production challenges, such as the requirement for glycosylation, development of a PFs48/45-based vaccine has been challenging. The present inventors have discovered that, surprisingly, minor modification of the protein sequence of Pfs48/45 6C results in a non-glycosylated, stabilized immunogen that retains a potent transmission blocking epitope and that has improved characteristics for vaccine manufacture. Thus, an invention of the disclosure may generally be practice by producing a Plasmodium 48/45 6C domain comprising mutations, such as those disclosed herein, that stabilize the 6C domain. In certain aspects, the modified 6C domain may be incorporated into nanoparticles that display the modified 6C domain. The modified 6C domain, or a nanoparticle displaying the modified domain, may be administered to an individual to elicit an immune response against Plasmodium sp. or spp, thereby protecting the individuals against, or treating them for, malaria.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the claims.

It must be noted that as used herein and in the appended claims, the singular forms “a,” “an,” and “the” include plural referents unless the context clearly dictates otherwise.

For example, a nucleic acid molecule refers to one or more nucleic acid molecules. As such, the terms “a”, “an”, “one or more” and “at least one” can be used interchangeably.

Similarly, the terms “comprising”, “including” and “having” can be used interchangeably. As used herein, the term “comprising” may be replaced with “consisting of” or with “consisting essentially of” in particular aspect, as desired. As used herein, the phrase “consisting essentially of” limits the scope of a claim to the specified materials or steps and those that do not materially affect the basic and novel characteristic(s) of the claimed invention.

It is further noted that the claims may be drafted to exclude any optional element. As such, this statement is intended to serve as antecedent basis for use of such exclusive terminology as “solely.” “only” and the like in connection with the recitation of claim elements, or use of a “negative” limitation.

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

As used herein, the term “about”, when used in relation to percent identity, means that the recited numerical value is approximate and small variations would not significantly affect the practice of the disclosed embodiments. Where a percent value is used, unless indicated otherwise by the context, the term “about” means the numerical value can vary by +5% and remain within the scope of the disclosed embodiments.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments are specifically embraced by the present invention and are disclosed herein just as if each and every combination was individually and explicitly disclosed. In addition, all sub-combinations are also specifically embraced by the present invention and are disclosed herein just as if each and every such sub-combination was individually and explicitly disclosed herein.

One aspect of the disclosure is modified 6C domain from a Plasmodium 48/45 protein (P48/45), wherein the amino acid sequence of the modified 6C domain has at least about 70%, at least about 72%, at least about 74%, at least about 75%, at least about 85%, at least about 90%, at least about 92%, at least about 94%, at least about 96%, at least about 98%, or at least about 99%, homology, or identity, over the entire length of the sequence of a wild-type (wt) Plasmodium 6C domain, wherein the amino acid sequence of the modified 6C domain may comprise one or more mutations relative to the amino acid sequence of the wt 6C domain that increase the stability of the modified 6C domain relative to the stability of the wt 6C domain, and wherein the modified 6C domain retains a mAb 85RF45.1 epitope such that mAb 85RF45.1, or a humanized version thereof, binds the modified 6C domain with high affinity. In certain aspects, the at least one of the one or more mutations are at an amino acid position selected from the group consisting of an amino acid position corresponding to S11 of SEQ ID NO:1, an amino acid position corresponding to S15 of SEQ ID NO:1, an amino acid position corresponding to D22 of SEQ ID NO:1, and an amino acid position corresponding to P69 of SEQ ID NO:1. In certain asepcts, the modified 6C domain comprises five or more mutations, wherein at least five of the mutations are at amino acid positions selected from the group consisting of an amino acid position corresponding to S11 of SEQ ID NO:1, an amino acid position corresponding to S15 of SEQ ID NO:1, an amino acid position corresponding to D22 of SEQ ID NO:1, an amino acid position corresponding to P44 of SEQ ID NO:1, an amino acid position corresponding to L52 of SEQ ID NO:1, an amino acid position corresponding to P69 of SEQ ID NO:1, and an amino acid position corresponding to G107 of SEQ ID NO:1. In certain aspects, the modified 6C domain comprises seven or more mutations, wherein at least seven of the mutations are at amino acid positions selected from the group consisting of an amino acid position corresponding to S11 of SEQ ID NO:1, an amino acid position corresponding to S15 of SEQ ID NO:1, an amino acid position corresponding to D22 of SEQ ID NO:1, an amino acid position corresponding to P44 of SEQ ID NO:1, an amino acid position corresponding to L52 of SEQ ID NO:1, an amino acid position corresponding to P69 of SEQ ID NO:1, and an amino acid position corresponding to G107 of SEQ ID NO:1.

As used herein, Plasmodium refers to the genus Plasmodium and encompasses all species of the genus. The P48/45 protein, and portions thereof, used in practicing aspects of the disclosure may be from any species of Plasmodium that causes illness, such as malaria. Examples of suitable Plasmodium species include, but are not limited to, P. falciparum (Pfs), P. vivax (Pvs), P. malariae (Pms), and P. ovale (Pov).

As used herein, the Plasmodium 48/45 protein (P48/45) refers to the six-cysteine, sexual stage-specific surface antigen of Plasmodium spp., one example of which is UniProt entry Q816T1. The 6C domain of P48/45 refers to the domain III (D3) of P48/45, which, as used herein refers to amino acid residues 291-429 of UniProt ID. Q8I6T1 (SEQ ID NO:27). The sequence of an exemplary 6C domain is represented by SEQ ID NO:1, which is the wild-type (wt) sequence of the 6C domain from Pfs. In certain aspects, the P48/45 may be from a Plasmodium (P.) species selected from the group consisting of P. falciparum (Pfs), P. vivax (Pvs), P. malariae (Pms), and P. ovale (Pov). In certain aspects, the P48/45 protein may be from Pfs or Pms. In certain aspects, the P48/45 6C domain may be from Pfs or Pms. In certain aspects, the P48/45 may be from Pfs. In certain aspects, the P48/45 6C domain may be from Pfs. In certain aspects, a wt Pfs48/45 6C domain may comprise, or consist of, SEQ ID NO:1.

A “modified 6C domain” refers to a 6C domain, the amino acid sequence of which has been modified from the wt 6C amino acid sequence by altering the sequence of the wt amino acid sequence by introducing mutations (e.g, substitutions, deletions, insertion) as specific amino acid positions in the wt 6C amino acid sequence to produce the modified 6C domain, Thus, the modified 6C domain may be described as being derived from the wt 6C domain. In certain aspects, a modified 6C domain may be produced as an isolated protein comprising the altered amino acid sequence.

As described above, a modified 6C domain of the disclosure has improved stability relative to the stability of a wt 6C domain. As used herein, stability refers to the ability to isolate high yields (e.g., >5 mg/L) of properly folded 6C domain, which is a function of the melting temperature (Tₘ) of the isolated 6C domain. Isolated wt 6C domain has a relatively low Tₘ and thus, purification of the wt 6C domain results in low yields (e.g., <3 mg/L). Modified 6C domains of the disclosure have a sufficiently high Tₘ such that high yields of properly folded modified 6C domain may be isolated from the production system. Thus, modified 6C domains of the disclosure have a Tₘ greater than the Tₘ of wt 6C domain and, consequently, the yield of modified 6C domain that may be obtained my purification is significantly greater than the yield of wt 6C domain that may be isolated using the same purification methodology. In certain aspects, the modified 6C domain has a Tₘ of at least 60° C., at least 62° C., at last 64° C., at least 66° C., at least 68° C., or at least 70° C. In certain aspects, the yield of modified 6C domain obtained may be at least twice (2×), at least 3×, at least 5×, at least 10×, at least 20×, or at least 25× the yield obtained using wt 6C domain, when the 6C domains are purified using the same purification conditions and methods. In certain aspects, at least 6 mg/L, at least about 8 mg/L, at least about 10 mg/L, at least about 20 mg/L, at least about 25 mg/L, at least about 50 mg/L or at least about 75 mg/L of modified 6C domain may be isolated.

As used herein, “properly folded” means that the modified 6C domain adopts a folded, tertiary structure that is identical, or nearly so, to the folded, tertiary structure of the wt 6C domain. In certain aspects, the “closeness’ of the modified 6C domain tertiary structure and the wt 6C tertiary structure may be compared by determining the free energy value of the modified 6C domain and the wt 6C domain. In certain aspects, the “closeness’ of the modified 6C domain tertiary structure and the wt 6C tertiary structure may be compared by measuring binding of an antibody to an epitope present in the 6C domain. In certain aspects, the antibody may be Fab85Rf45.1, which binds a “85RF45.1 epitope” present in the 6C domain with high affinity.

As used herein, “epitope” refers to the location on a protein (antigen) that is bound by an antibody. Amino acid residues (specificity determining residues (SDRs) in the antibody complementary determining regions (CDRs), interact with specific amino acid residues in the epitope, called contact residues, resulting in binding of the antibody to the epitope. An epitope may comprise contiguous amino acid residues that interact with the antibody, discontiguous amino acid residues that interact with the antibody, or combinations thereof. In epitopes comprising discontiguous amino acid residues, folding of the protein brings the contact residues into proximity to one another, thereby allowing the specificity determining residues in the antibody to bind contact residues from parts of the antigen that are far apart in the linear amino acid sequence of the antigen.

As used herein, “85RF45.1 epitope” is an epitope recognized (i.e., bound) by the monoclonal antibody mAb 85RF45.1. The mAb 85RF45.1 epitope is normally present in wt P48/45 6C domain and Fab85RF45.1 binds the 85RF45 epitope in the P48/45 6C domain with high affinity (K_{D}>10⁻⁹). Because P48/45 is expressed on gametocytes, binding of Fab85Rf45.1 prevents fertilization in the mosquito midgut, thereby blocking transmission of the parasite. In certain aspects, mAb 85RF45.1 binds the modified 6C domain with a K_{D} of at least 10⁻⁶, 10⁻⁷, 10⁻⁸, 10⁻⁹, 10⁻¹⁰, 10⁻¹¹, or 10⁻¹². In certain aspects, the contact residues of the 85RF45.1 epitope comprise one or more amino acid residues selected from the group consisting of D31, P55, G56, D57, 158, 159, P60, D61, F64, Q65, L74, E75, P76, S77, 179, Y81, E95, D100, K102, K104, 1121, K123, K124, D125, K126, and S128 of SEQ ID NO:1.

In certain aspects, the contact residues of the 85RF45.1 epitope comprise, or consist of, at least 10 amino acid residues, at least 15 amino acid residues, at least 20 amino acid residues, at least 21 amino acid residues, at least 22 amino acid residues, at least 23 amino acid residues, at least 24 amino acid residues, or at least 25 amino acid residues selected from the group consisting of an aspartic acid at a position corresponding to position 31 of SEQ ID NO:1 [D321], a proline at a position corresponding to position 55 of SEQ ID NO:1 [P345], a glycine at a position corresponding to position 56 of SEQ ID NO:1 [G346], an aspartic acid at a position corresponding to position 57 of SEQ ID NO:1 [D347], a isoleucine at a position corresponding to position 58 of SEQ ID NO:1 [1348], a isoleucine at a position corresponding to position 59 of SEQ ID NO:1 [1349], a proline at a position corresponding to position 60 of SEQ ID NO:1 [P350], an aspartic acid at a position corresponding to position 61 of SEQ ID NO:1 [D351], a phenylalanine at a position corresponding to position 64 of SEQ ID NO:1 [F354], a glutamine at a position corresponding to position 65 of SEQ ID NO:1 [Q355], a leucine at a position corresponding to position 74 of SEQ ID NO:1 [L364], a glutamic acid at a position corresponding to position 75 of SEQ ID NO:1 [E365], a proline at a position corresponding to position 76 of SEQ ID NO:1 [P366], a serine at a position corresponding to position 77 of SEQ ID NO:1 [P367], an isoleucine at a position corresponding to position 79 of SEQ ID NO:1 [1369], a tyrosine at apposition corresponding to position 81 of SEQ ID NO: 1 [Y371], a glutamic acid at a position corresponding to position 95 of SEQ ID NO:1 [E385], an aspartic acid a position corresponding to position 100 of SEQ ID NO:1 [D390], a lysine at a position corroding to position 102 of SEQ ID NO:1 [K392], a lysine at a position corresponding to position 104 of SEQ ID NO:1 [K394], an isoleucine at a position corresponding to position 121 of SEQ ID NO:1 [1411], a lysine at a position corresponding to position 123 of SEQ ID NO: 1 [K413], a lysine at a position corresponding to position 124 of SEQ ID NO:1 [K414], an aspartic acid at a position corresponding to position 125 of SEQ ID NO:1 [D415], a lysine at a position corresponding to position 126 of SEQ ID NO:1 [K416], and a serine at a position corresponding to position 128 of SEQ ID NO: 1 [S418]. In certain aspects, the contact residues of the 85RF45.1 epitope comprise or consist of, at least 10 amino acid residues, at least 15 amino acid residues, at least 20 amino acid residues, at least 21 amino acid residues, at least 22 amino acid residues, at least 23 amino acid residues, at least 24 amino acid residues, or at least 25 amino acid residues selected from the group consisting of D31, P55, G56, D57, 158, 159, P60, D61, F64, Q65, L74, E75, P76, S77, 179, Y81, E95, D100, K102, K104, 1121, K123, K124, D125, K126, and S128 of SEQ ID NO:1 In certain aspects, the contact residues of the 85RF45.1 epitope comprise, or consist of, D31, P55, G56, D57, 158, 159, P60, D61, F64, Q65, L74, E75, P76, S77, 179, Y81, K102, K104, 1121, K123, K124, D125, K126, and S128 of SEQ ID NO:1. In certain aspects, the contact residues may also, optionally, comprise E95 and/or D100, of SEQ ID NO:1. In certain asepcts, the mutations in the modified 6C domain are not at a position comprising a contact residue for mAb 85RF45.1, or a humanized version thereof.

As used herein, “corresponding” refers to the position in a first amino acid sequence relative to the position of a second amino acid residue in a reference sequence, when the first amino acid sequence and the reference amino acid sequence are aligned to yield the maximum percent identity. For example, when the amino acid sequences ALWXFGS (SEQ ID NO:28) and ALWYFG (SEQ ID NO:29) are aligned, the position of X in SEQ ID NO:28 (i.e., position 4) corresponds to the position of Y, which is position 4, in SEQ ID NO:29. It will be understood by those of skill in the art that, due to insertions, deletions, truncations and substitution mutations, two sequences being aligned may not be exactly the same length or share 100% identity. Thus, corresponding positions in the two sequences may not be in the exact same numerical position in the linear sequence. In such instances, the first amino acid position may be shifted by one or two amino acid positions in either direction. For example, when the amino acid sequences ALWXFGS (SEQ ID NO:28) and ALWAYFGS (SEQ ID NO: 30) are aligned, the position of X in SEQ ID NO:28 (i.e., position 4) corresponds to the position of Y, which is position 5 in SEQ ID NO:30. The concept of corresponding amino acid residues is understood by those of skill in the art. Certain disclosures herein refer to an amino acid position as corresponding to an amino acid position in a SEQ ID NO (e.g., SEQ ID NO:1). In certain disclosures, the recitation also contains a bolded amino acid and position number (e.g., D321). It should be understood that the bolded amino acid/position designation refers to the amino acid position in the full length Pfs P48/45 protein (SEQ ID NO:27).

In certain aspects, a modified 6C domain of the disclosure may comprise at least 10 amino acid residues, at least 15 amino acid residues, at least 20 amino acid residues, at least 21 amino acid residues, at least 22 amino acid residues, at least 23 amino acid residues, at least 24 amino acid residues, or at least 25 amino acid residues selected from the group consisting of an aspartic acid at a position corresponding to position 31 of SEQ ID NO:1 [D321], a proline at a position corresponding to position 55 of SEQ ID NO:1 [P345], a glycine at a position corresponding to position 56 of SEQ ID NO:1 [G346], an aspartic acid at a position corresponding to position 57 of SEQ ID NO:1 [D347], a isoleucine at a position corresponding to position 58 of SEQ ID NO:1 [1348], a isoleucine at a position corresponding to position 59 of SEQ ID NO:1 [1349], a proline at a position corresponding to position 60 of SEQ ID NO:1 [P350], an aspartic acid at a position corresponding to position 61 of SEQ ID NO:1 [D351], a phenylalanine at a position corresponding to position 64 of SEQ ID NO:1 [F354], a glutamine at a position corresponding to position 65 of SEQ ID NO:1 [Q355], a leucine at a position corresponding to position 74 of SEQ ID NO:1 [L364], a glutamic acid at a position corresponding to position 75 of SEQ ID NO:1 [E365], a proline at a position corresponding to position 76 of SEQ ID NO:1 [P366], a serine at a position corresponding to position 77 of SEQ ID NO:1 [P367], an isoleucine at a position corresponding to position 79 of SEQ ID NO:1 [1369], a tyrosine at apposition corresponding to position 81 of SEQ ID NO: 3 [Y371], a glutamic acid at a position corresponding to position 95 of SEQ ID NO:1 [E385], an aspartic acid a position corresponding to position 100 of SEQ ID NO:1 [D390], a lysine at a position corroding to position 102 of SEQ ID NO:1 [K392], a lysine at a position corresponding to position 104 of SEQ ID NO:1 [K394], an isoleucine at a position corresponding to position 121 of SEQ ID NO:1 [1411], a lysine at a position corresponding to position 123 of SEQ ID NO: 1 [K413], a lysine at a position corresponding to position 124 of SEQ ID NO:1 [K414], an aspartic acid at a position corresponding to position 125 of SEQ ID NO:1 [D415], a lysine at a position corresponding to position 126 of SEQ ID NO:1 [K416], and a serine at a position corresponding to position 128 of SEQ ID NO:1 [S418].

In certain aspects, a modified 6C domain of the disclosure may comprise an aspartic acid at a position corresponding to position 31 of SEQ ID NO:1 [D321], a proline at a position corresponding to position 55 of SEQ ID NO:1 [P345], a glycine at a position corresponding to position 56 of SEQ ID NO:1 [G346], an aspartic acid at a position corresponding to position 57 of SEQ ID NO:1 [D347], a isoleucine at a position corresponding to position 58 of SEQ ID NO:1 [1348], a isoleucine at a position corresponding to position 59 of SEQ ID NO:1 [1349], a proline at a position corresponding to position 60 of SEQ ID NO:1 [P350], an aspartic acid at a position corresponding to position 61 of SEQ ID NO:1 [D351], a phenylalanine at a position corresponding to position 64 of SEQ ID NO:1 [F354], a glutamine at a position corresponding to position 65 of SEQ ID NO:1 [Q355], a leucine at a position corresponding to position 74 of SEQ ID NO:1 [L364], a glutamic acid at a position corresponding to position 75 of SEQ ID NO:1 [E365], a proline at a position corresponding to position 76 of SEQ ID NO:1 [P366], a serine at a position corresponding to position 77 of SEQ ID NO:1 [P367], an isoleucine at a position corresponding to position 79 of SEQ ID NO:1 [1369], a tyrosine at apposition corresponding to position 81 of SEQ ID NO:1 [Y371], a lysine at a position corroding to position 102 of [K392], a lysine at a position corresponding to position 104 of SEQ ID NO: 1 [K394], an isoleucine at a position corresponding to position 121 of SEQ ID NO:1 [I411], a lysine at a position corresponding to position 123 of SEQ ID NO:1 [K413], a lysine at a position corresponding to position 124 of SEQ ID NO:1 [K414], an aspartic acid at a position corresponding to position 125 of SEQ ID NO:1 [D415], a lysine at a position corresponding to position 126 of SEQ ID NO:1 [K416], and a serine at a position corresponding to position 128 of SEQ ID NO:1 [S418]. In certain aspects, a modified 6C domain of the disclosure may also, optionally, comprise a glutamic acid at a position corresponding to position 95 of SEQ ID NO:1 [E385] and/or an aspartic acid a position corresponding to position 100 of SEQ ID NO:1 [D390].

As stated above, modified 6C domains of the disclosure comprise one or more mutations, relative to the amino acid sequence of the wt 6C domain, that increase the stability of the modified 6C domain relative to the stability of the wt 6C domain. Such mutations may comprise insertions, deletions, and substitutions, which may comprise conservative substitutions. In certain aspects, the mutations comprise substitution mutations, which may comprise conservative substitution mutations. The one or more mutations may be made, at any positions in the 6C domain, so long as the one or more mutations do not significantly alter the ability of mAb 85RF45.1 to bind the 85RF451 epitope in the modified 6C domain. Preferably, mutations are not made at a significant number of positions, or at any position, containing a contact residue, although mutations may be made at a few (e.g., 1, 2 or 3) positions containing contact residues without significantly affecting binding of Fab85Rf45′0.1 to the 85Rf45.1 epitope. As used herein, “significantly affect binding” means that the amino acid change reduces the K_{d} of Fab85Rf45′0.1 for the 85Rf45.1 epitope by at least 10^{-0.5} or at least 10⁻¹.

In certain aspects, the modified 6C domain does not contain a mutation in at least 10 amino acid positions, in at least 15 an amino acid positions, in at least at least 20 an amino acid positions, in at least 21 an amino acid positions, in at least 22 an amino acid positions, in at least 23 amino acid positions, or in at least 24 positions selected from the group consisting of an amino acid position corresponding to position 31 of SEQ ID NO:1, an amino acid position corresponding to position 55 of SEQ ID NO: 1, an amino acid position corresponding to position 56 of SEQ ID NO:1, an amino acid position corresponding to position 57 of SEQ ID NO:1, an amino acid position corresponding to position 58 of SEQ ID NO:1, an amino acid position corresponding to position 59 of SEQ ID NO:1, an amino acid position corresponding to position 60 of SEQ ID NO:1, an amino acid position corresponding to position 61 of SEQ ID NO:1, an amino acid position corresponding to position 64 of SEQ ID NO:1, an amino acid position corresponding to position 65 of SEQ ID NO:1, an amino acid position corresponding to position 74 of SEQ ID NO:1, an amino acid position corresponding to position 77 of SEQ ID NO:1, an amino acid position corresponding to position 79 of SEQ ID NO:1, an amino acid position corresponding to position 80 of SEQ ID NO:1, an amino acid position corresponding to position 81 of SEQ ID NO:1, an amino acid position corresponding to position 90 of SEQ ID NO:1, an amino acid position corresponding to position 102 of SEQ ID NO:1, an amino acid position corresponding to position 104 of SEQ ID NO:1, an amino acid position corresponding to position 110 of SEQ ID NO:1, an amino acid position corresponding to position 121 of SEQ ID NO:1, an amino acid position corresponding to position 123 of SEQ ID NO:1, an amino acid position corresponding to position 124 of SEQ ID NO:1, an amino acid position corresponding to position 125 of SEQ ID NO:1, an amino acid position corresponding to position 126 of SEQ ID NO:1, and an amino acid position corresponding to position 128 of SEQ ID NO:1. Optionally, the modified 6C domain does not contain a mutation at an amino acid position corresponding to position 95 of SEQ ID NO:1 and/or an amino acid position corresponding to position 100 of SEQ ID NO:1.

In certain aspects, the modified 6C domain may comprise a mutation at one or more amino acid positions selected from the group consisting of an amino acid position corresponding to position 3 of SEQ ID NO:1, an amino acid position corresponding to position 6 of SEQ ID NO:1, an amino acid position corresponding to position 11 of SEQ ID NO:1, an amino acid position corresponding to position 14 of SEQ ID NO:1, an amino acid position corresponding to position 15 of SEQ ID NO:1, an amino acid position corresponding to position 18 of SEQ ID NO:1, an amino acid position corresponding to position 19 of SEQ ID NO:1, an amino acid position corresponding to position 21 of SEQ ID NO:1, an amino acid position corresponding to position 22 of SEQ ID NO:1, an amino acid position corresponding to position 28 of SEQ ID NO:1, an amino acid position corresponding to position 33 of SEQ ID NO:1, an amino acid position corresponding to position 35 of SEQ ID NO:1, an amino acid position corresponding to position 36 of SEQ ID NO:1, an amino acid position corresponding to position 38 of SEQ ID NO:1, an amino acid position corresponding to position 40 of SEQ ID NO:1, an amino acid position corresponding to position 43 of SEQ ID NO:1, an amino acid position corresponding to position 44 of SEQ ID NO:1, an amino acid position corresponding to position 45 of SEQ ID NO:1, an amino acid position corresponding to position 49 of SEQ ID NO:1, an amino acid position corresponding to position 52 of SEQ ID NO:1, an amino acid position corresponding to position 68 of SEQ ID NO:1, an amino acid position corresponding to position 69 of SEQ ID NO:1, an amino acid position corresponding to position 70 of SEQ ID NO:1, an amino acid position corresponding to position 71 of SEQ ID NO:1, an amino acid position corresponding to position 83 of SEQ ID NO:1, an amino acid position corresponding to position 84 of SEQ ID NO:1, an amino acid position corresponding to position 85 of SEQ ID NO:1, an amino acid position corresponding to position 86 of SEQ ID NO:1, an amino acid position corresponding to position 89 of SEQ ID NO:1, an amino acid position corresponding to position 90 of SEQ ID NO:1, an amino acid position corresponding to position 97 of SEQ ID NO:1, an amino acid position corresponding to position 105 of SEQ ID NO:1, an amino acid position corresponding to position 107 of SEQ ID NO:1, an amino acid position corresponding to position 109 of SEQ ID NO:1, an amino acid position corresponding to position 114 of SEQ ID NO:1, an amino acid position corresponding to position 115 of SEQ ID NO:1, an amino acid position corresponding to position 134 of SEQ ID NO:1, and an amino acid position corresponding to position 138 of SEQ ID NO:1.

### Immunogen 15

In certain aspects, the modified 6C domain may comprise a mutation at one or more amino acid positions selected from the group consisting of an amino acid position corresponding to position 3 of SEQ ID NO:1, an amino acid position corresponding to position 6 of SEQ ID NO:1, an amino acid position corresponding to position 11 of SEQ ID NO:1, an amino acid position corresponding to position 14 of SEQ ID NO:1, an amino acid position corresponding to position 15 of SEQ ID NO:1, an amino acid position corresponding to position 18 of SEQ ID NO:1, an amino acid position corresponding to position 19 of SEQ ID NO:1, an amino acid position corresponding to position 21 of SEQ ID NO:1, an amino acid position corresponding to position 22 of SEQ ID NO:1, an amino acid position corresponding to position 28 of SEQ ID NO:1, an amino acid position corresponding to position 33 of SEQ ID NO:1, an amino acid position corresponding to position 35 of SEQ ID NO:1, an amino acid position corresponding to position 36 of SEQ ID NO:1, an amino acid position corresponding to position 38 of SEQ ID NO:1, an amino acid position corresponding to position 43 of SEQ ID NO:1, an amino acid position corresponding to position 45 of SEQ ID NO:1, an amino acid position corresponding to position 49 of SEQ ID NO:1, an amino acid position corresponding to position 68 of SEQ ID NO:1, an amino acid position corresponding to position 69 of SEQ ID NO:1, an amino acid position corresponding to position 70 of SEQ ID NO:1, an amino acid position corresponding to position 71 of SEQ ID NO:1, an amino acid position corresponding to position 83 of SEQ ID NO:1, an amino acid position corresponding to position 84 of SEQ ID NO:1, an amino acid position corresponding to position 86 of SEQ ID NO:1, an amino acid position corresponding to position 89 of SEQ ID NO:1, an amino acid position corresponding to position 90 of SEQ ID NO:1, an amino acid position corresponding to position 97 of SEQ ID NO:1, an amino acid position corresponding to position 105 of SEQ ID NO:1, an amino acid position corresponding to position 107 of SEQ ID NO:1, an amino acid position corresponding to position 109 of SEQ ID NO:1, an amino acid position corresponding to position 114 of SEQ ID NO:1, an amino acid position corresponding to position 115 of SEQ ID NO:1, an amino acid position corresponding to position 134 of SEQ ID NO:1, and an amino acid position corresponding to position 138 of SEQ ID NO:1.

In certain aspects, the modified 6C domain may comprise one or more amino acids selected from the group consisting of a serine at a position corresponding to position 3 of SEQ ID NO:1, an alanine at a position corresponding to position 6 of SEQ ID NO:1, an alanine at a position corresponding to position 11 of SEQ ID NO: 1, a lysine at a position corresponding to position 14 of SEQ ID NO:1, an asparagine at a position corresponding to position 15 of SEQ ID NO:1, a tyrosine at a position corresponding to position 18 of SEQ ID NO:1, a lysine at a position corresponding to position 19 of SEQ ID NO:1, an alanine at a position corresponding to position 21 of SEQ ID NO:1, a lysine at a position corresponding to position 22 of SEQ ID NO: 1, a glutamine at a position corresponding to position 28 of SEQ ID NO:1, a lysine at a position corresponding to position 33 of SEQ ID NO:1, a valine at a position corresponding to position 35 of SEQ ID NO:1, an as arginine at a position corresponding to position 36 of SEQ ID NO:1, a glutamic acid at a position corresponding to position 38 of SEQ ID NO:1, a histidine at a position corresponding to position 43 of SEQ ID NO: 1, a glutamic acid at a position corresponding to position 45 of SEQ ID NO:1, an isoleucine at a position corresponding to position 49 of SEQ ID NO:1, histidine at a position corresponding to position 68 of SEQ ID NO:1, an asparagine at a position corresponding to position 69 of SEQ ID NO:1, a serine at a position corresponding to position 70 of SEQ ID NO:1, an alanine at a position corresponding to position 71 of SEQ ID NO:1, a glutamic acid at a position corresponding to position 83 of SEQ ID NO: 1, as aspartic acid at a position corresponding to position 84 of SEQ ID NO:1, a leucine at a position corresponding to position 86 of SEQ ID NO:1, an alanine at a position corresponding to position 89 of SEQ ID NO:1, an asparagine at a position corresponding to position 90 of SEQ ID NO:1, an arginine at a position corresponding to position 97 of SEQ ID NO:1, a methionine at a position corresponding to position 105 of SEQ ID NO:1, an alanine at a position corresponding to position 107 of SEQ ID NO:1, an isoleucine at a position corresponding to position 109 of SEQ ID NO:1, a glutamic acid at a position corresponding to position 114 of SEQ ID NO:1, a proline at a position corresponding to position 115 of SEQ ID NO:1, a phenylalanine at a position corresponding to position 134 of SEQ ID NO:1, and a serine at a position corresponding to position 138 of SEQ ID NO:1.

In certain aspects, the modified 6C domain may comprise at least 10 amino acid residues, at least 15 amino acid residues, at least 20 amino acid residues, at least 25 amino acid residues, at least 30 amino acid residues, at least 21 amino acid residues, at least 32 amino acid residues, or at least 33 amino acid residues selected from the group consisting of S3, A6, A11, K14, N15, Y18, K19, A21, K22, Q28, K33, V35, R36, E38, H43, E45, 149, H68, N69, S70, A71, E83, D84, L86, A89, N90, R97, M105, A107, 1109, E114, P115, E134, and S138, of SEQ ID NO:2. In certain aspects, the modified 6C domain may comprise S3, A6, A11, K14, N15, Y18, K19, A21, K22, Q28, K33, V35, R36, E38, H43, E45, 149, H68, N69, S70, A71, E83, D84, L86, A89, N90, R97, M105, A107, 1109, E114, P115, E134, and S138, of SEQ ID NO:2, and, optionally, E95 and/or D100, of SEQ ID NO:2. In certain aspects, the modified 6C domain may comprise one or more amino acid residues selected from the group consisting of a serine at position 3, an alanine at position 6, an alanine at position 11, a lysine at position 14, an asparagine at position 15, a tyrosine at position 18, a lysine at position 19, an alanine at position 21, a lysine at position 22, a glutamine at position 28, a lysine at position 33, a valine at position 35, an as arginine at position 36, a glutamic acid at position 38, a histidine at position 43, a glutamic acid at position 45, an isoleucine at position 49, histidine at position 68, an asparagine at position 69, a serine at position 70, an alanine at position 71, a glutamic acid at position 83, as aspartic acid at position 84, a leucine at position 86, an alanine at position 89, an asparagine at position 90, an arginine at position 97, a methionine at position 105, an alanine at position 107, an isoleucine at position 109, a glutamic acid at position 114, a proline at position 115, a phenylalanine at position 134, and a serine at position 138. In certain aspects, the amino acid sequence may comprise a serine at position 3, an alanine at position 6, an alanine at position 11, a lysine at position 14, an asparagine at position 15, a tyrosine at position 18, a lysine at position 19, an alanine at position 21, a lysine at position 22, a glutamine at position 28, a lysine at position 33, a valine at position 35, an as arginine at position 36, a glutamic acid at position 38, a histidine at position 43, a glutamic acid at position 45, an isoleucine at position 49, histidine at position 68, an asparagine at position 69, a serine at position 70, an alanine at position 71, a glutamic acid at position 83, as aspartic acid at position 84, a leucine at position 86, an alanine at position 89, an asparagine at position 90, an arginine at position 97, a methionine at position 105, an alanine at position 107, an isoleucine at position 109, a glutamic acid at position 114, a proline at position 115, a phenylalanine at position 134, and a serine at position 138. In certain aspects, the amino acid sequence lacks an n-glycosylation signal. In certain aspects, the amino acid sequence may comprise SEQ ID NO:2.

### Immunogen 15-2

In certain aspects, the modified 6C domain may comprise a mutation at one or more amino acid positions selected from the group consisting of an amino acid position corresponding to position 6 of SEQ ID NO:1, an amino acid position corresponding to position 11 of SEQ ID NO:1, an amino acid position corresponding to position 14 of SEQ ID NO:1, an amino acid position corresponding to position 15 of SEQ ID NO:1, an amino acid position corresponding to position 18 of SEQ ID NO:1, an amino acid position corresponding to position 19 of SEQ ID NO:1, an amino acid position corresponding to position 22 of SEQ ID NO:1, an amino acid position corresponding to position 33 of SEQ ID NO:1, an amino acid position corresponding to position 36 of SEQ ID NO:1, an amino acid position corresponding to position 38 of SEQ ID NO:1, an amino acid position corresponding to position 43 of SEQ ID NO:1, an amino acid position corresponding to position 49 of SEQ ID NO:1, an amino acid position corresponding to position 69 of SEQ ID NO:1, an amino acid position corresponding to position 70 of SEQ ID NO:1, an amino acid position corresponding to position 71 of SEQ ID NO:1, an amino acid position corresponding to position 83 of SEQ ID NO:1, an amino acid position corresponding to position 86 of SEQ ID NO:1, an amino acid position corresponding to position 89 of SEQ ID NO:1, an amino acid position corresponding to position 97 of SEQ ID NO:1, an amino acid position corresponding to position 105 of SEQ ID NO:1, an amino acid position corresponding to position 107 of SEQ ID NO:1, an amino acid position corresponding to position 115 of SEQ ID NO:1, and an amino acid position corresponding to position 134 of SEQ ID NO: 1.

In certain aspects, the modified 6C domain may comprise one or more amino acids selected from the group consisting of an alanine at a position corresponding to position 6 of SEQ ID NO:1, an alanine at a position corresponding to position 11 of SEQ ID NO:1, a lysine at a position corresponding to position 14 of SEQ ID NO: 1, an alanine at a position corresponding to position 15 of SEQ ID NO:1, a tyrosine at a position corresponding to position 18 of SEQ ID NO:1, a lysine at a position corresponding to position 19 of SEQ ID NO:1, a lysine at a position corresponding to position 22 of SEQ ID NO:1, a lysine at a position corresponding to position 33 of SEQ ID NO: 1, an arginine at a position corresponding to position 36 of SEQ ID NO:1, a glutamic acid at a position corresponding to position 38 of SEQ ID NO:1, a histidine at a position corresponding to position 43 of SEQ ID NO:1, an isoleucine at a position corresponding to position 49 of SEQ ID NO:1, an asparagine at a position corresponding to position 69 of SEQ ID NO:1, a serine at a position corresponding to position 70 of SEQ ID NO:1, an alanine at a position corresponding to position 71 of SEQ ID NO:1, a glutamic acid at a position corresponding to position 83 of SEQ ID NO:1, a leucine at a position corresponding to position 86 of SEQ ID NO:1, an alanine at a position corresponding to position 89 of SEQ ID NO:1, an arginine at a position corresponding to position 97 of SEQ ID NO:1, a methionine at a position corresponding to position 105 of SEQ ID NO:1, an alanine at a position corresponding to position 107 of SEQ ID NO:1, a proline at a position corresponding to position 115 of SEQ ID NO:1, and a phenylalanine at a position corresponding to position 134 of SEQ ID NO:1.

In certain aspects, the modified 6C domain may comprise at least 10 amino acid residues, at least 15 amino acid residues, at least 20 amino acid residues, at least 21 amino acid residues, or at least 22 amino acid residues selected from the group consisting of one or more amino acid residues selected from the group consisting of A6, A11, K14, A15, Y18, K19, K22, K33, R36, E38, H43, 149, N69, S70, A71, E83, L86, A89, R97, M105, A107, P115, F134, of SEQ ID NO:3. In certain aspects, the modified 6C domain may comprise A6, A11, K14, A15, Y18, K19, K22, K33, R36, E38, H43, 149, N69, S70, A71, E83, L86, A89, R97, M105, A107, P115, F134, of SEQ ID NO:3. In certain aspects, the modified 6C domain may comprise one or more amino acids selected from the group consisting of an alanine at position 6, an alanine at position 11, a lysine at position 14, an alanine at position 15, a tyrosine at position 18, a lysine at position 19, a lysine at position 22, a lysine at position 33, an arginine at position 36, a glutamic acid at position 38, a histidine at position 43, an isoleucine at position 49, an asparagine at position 69, a serine at position 70, an alanine at position 71, a glutamic acid at a position corresponding to position 83, a leucine at position 86, an alanine at position 89, an arginine at position 97, a methionine at position 105, an alanine at position 107, a proline at position 115, and a phenylalanine at position 134. In certain aspects, the amino acid sequence may comprise an alanine at position 6, an alanine at position 11, a lysine at position 14, an alanine at position 15, a tyrosine at position 18, a lysine at position 19, a lysine at position 22, a lysine at position 33, an arginine at position 36, a glutamic acid at position 38, a histidine at position 43, an isoleucine at position 49, an asparagine at position 69, a serine at position 70, an alanine at position 71, a glutamic acid at a position corresponding to position 83, a leucine at position 86, an alanine at position 89, an arginine at position 97, a methionine at position 105, an alanine at position 107, a proline at position 115, and a phenylalanine at position 134. In certain aspects, the modified 6C domain may comprise SEQ ID NO:3.

### Immunogen 17

In certain aspects, the modified 6C domain may comprise a mutation at one or more amino acid positions selected from the group consisting of an amino acid position corresponding to position 3 of SEQ ID NO:1, an amino acid position corresponding to position 11 of SEQ ID NO:1, an amino acid position corresponding to position 14 of SEQ ID NO:1, an amino acid position corresponding to position 15 of SEQ ID NO:1, an amino acid position corresponding to position 19 of SEQ ID NO:1, an amino acid position corresponding to position 21 of SEQ ID NO:1, an amino acid position corresponding to position 22 of SEQ ID NO:1, an amino acid position corresponding to position 28 of SEQ ID NO:1, an amino acid position corresponding to position 33 of SEQ ID NO:1, an amino acid position corresponding to position 35 of SEQ ID NO:1, an amino acid position corresponding to position 36 of SEQ ID NO:1, an amino acid position corresponding to position 38 of SEQ ID NO:1, an amino acid position corresponding to position 40 of SEQ ID NO:1, an amino acid position corresponding to position 43 of SEQ ID NO:1, an amino acid position corresponding to position 44 of SEQ ID NO:1, an amino acid position corresponding to position 45 of SEQ ID NO:1, an amino acid position corresponding to position 49 of SEQ ID NO:1, an amino acid position corresponding to position 52 of SEQ ID NO:1,an amino acid position corresponding to position 68 of SEQ ID NO:1, an amino acid position corresponding to position 69 of SEQ ID NO:1, an amino acid position corresponding to position 70 of SEQ ID NO:1, an amino acid position corresponding to position 84 of SEQ ID NO:1, an amino acid position corresponding to position 85 of SEQ ID NO:1, an amino acid position corresponding to position 89 of SEQ ID NO:1, an amino acid position corresponding to position 97 of SEQ ID NO:1, an amino acid position corresponding to position 107 of SEQ ID NO:1, an amino acid position corresponding to position 115 of SEQ ID NO:1, an amino acid position corresponding to position 134 of SEQ ID NO:1, and an amino acid position corresponding to position 138 of SEQ ID NO: 1.

In certain aspects, the modified 6C domain may comprise one or more amino acids selected from the group consisting of a serine at a position corresponding to position 3 of SEQ ID NO:1, an alanine at a position corresponding to position 11 of SEQ ID NO:1, a lysine at a position corresponding to position 14 of SEQ ID NO: 1, an asparagine at a position corresponding to position 15 of SEQ ID NO:1, a lysine at a position corresponding to position 19 of SEQ ID NO:1, an alanine at a position corresponding to position 21 of SEQ ID NO:1, a lysine at a position corresponding to position 22 of SEQ ID NO:1, a glutamine at a position corresponding to position 28 of SEQ ID NO:1, a lysine at a position corresponding to position 33 of SEQ ID NO: 1, a valine at a position corresponding to position 35 of SEQ ID NO:1, an arginine at a position corresponding to position 36 of SEQ ID NO:1, a glutamic acid at a position corresponding to position 38 of SEQ ID NO:1, a glutamic acid at a position corresponding to position 40 of SEQ ID NO:1, a histidine at a position corresponding to position 43 of SEQ ID NO:1, a lysine at a position corresponding to position 44 of SEQ ID NO: 1, a tyrosine at a position corresponding to position 45 of SEQ ID NO:1, an isoleucine at a position corresponding to position 49 of SEQ ID NO:1, a methionine at a position corresponding to position 52 of SEQ ID NO:1, a histidine at a position corresponding to position 68 of SEQ ID NO:1, a glutamine at a position corresponding to position 69 of SEQ ID NO:1, a serine at a position corresponding to position 70 of SEQ ID NO: 1, as aspartic acid at a position corresponding to position 84 of SEQ ID NO:1, a valine at a position corresponding to position 85 of SEQ ID NO:1, an aspartic acid at a position corresponding to position 89 of SEQ ID NO:1, an arginine at a position corresponding to position 97 of SEQ ID NO:1, a lysine at a position corresponding to position 107 of SEQ ID NO:1, a proline at a position corresponding to position 115 of SEQ ID NO:1, a lysine at a position corresponding to position 134 of SEQ ID NO:1, and a serine at a position corresponding to position 138 of SEQ ID NO:1.

In certain aspects, the modified 6C domain may comprise at least 10 amino acid residues, at least 15 amino acid residues, at least 20 amino acid residues, at least 25 amino acid residues, at least 26 amino acid residues, at least 27 amino acid residues, or at least 28 amino acid residues selected from the group consisting of S3, A11, K14, N15, K19, A21, K22, Q28, K33, V35, R36, E38, E40, H43, K44, Y45, 149, M52, H68, Q69, S70, D84, V85, D89, R97, K107, P115, K134, S138, of SEQ ID NO:4. In certain aspects, the modified 6C domain may comprise S3, A11, K14, N15, K19, A21, K22, Q28, K33, V35, R36, E38, E40, H43, K44, Y45, 149, M52, H68, Q69, S70, D84, V85, D89, R97, K107, P115, K134, S138, of SEQ ID NO:4. In certain aspects, the amino acid sequence may comprise one or more amino acids selected from the group consisting of a serine at position 3, an alanine at position 11, a lysine at position 14, an asparagine at position 15, a lysine at position 19, an alanine at position 21, a lysine at position 22, a glutamine at position 28, a lysine at position 33, a valine at position 35, an arginine at position 36, a glutamic acid at position 38, a glutamic acid at position 40, a histidine at position 43, a lysine at position 44, a tyrosine at position 45, an isoleucine at position 49, a methionine at position 52, a histidine at position 68, a glutamine at position 69, a serine at position 70, as aspartic acid at position 84, a valine at position 85, an aspartic acid at position 89, an arginine at position 97, a lysine at position 107, a proline at position 115, a lysine at position 134, and a serine at position 138. In certain aspects, the amino acid sequence may comprise a serine at position 3, an alanine at position 11, a lysine at position 14, an asparagine at position 15, a lysine at position 19, an alanine at position 21, a lysine at position 22, a glutamine at position 28, a lysine at position 33, a valine at position 35, an arginine at position 36, a glutamic acid at position 38, a glutamic acid at position 40, a histidine at position 43, a lysine at position 44, a tyrosine at position 45, an isoleucine at position 49, a methionine at position 52, a histidine at position 68, a glutamine at position 69, a serine at position 70, as aspartic acid at position 84, a valine at position 85, an aspartic acid at position 89, an arginine at position 97, a lysine at position 107, a proline at position 115, a lysine at position 134, and a serine at position 138. In certain aspects, the amino acid sequence may comprise SEQ ID NO:4.

### Immunogen 17-4

In certain aspects, the modified 6C domain may comprise mutations at one or more amino acid positions selected from the group consisting of an amino acid position corresponding to position 11 of SEQ ID NO:1, an amino acid position corresponding to position 15 of SEQ ID NO:1, an amino acid position corresponding to position 22 of SEQ ID NO:1, an amino acid position corresponding to position 44 of SEQ ID NO:1, an amino acid position corresponding to position 52 of SEQ ID NO:1, an amino acid position corresponding to position 69 of SEQ ID NO:1, and an amino acid position corresponding to position 107 of SEQ ID NO:1.

In certain aspects, the modified 6C domain may comprise one or more amino acid positions selected from the group consisting of an alanine at a position corresponding to position 11 of SEQ ID NO:1, an alanine at a position corresponding to position 15 of SEQ ID NO:1, a lysine at a position corresponding to position 22 of SEQ ID NO: 1, a lysine at a position corresponding to position 44 of SEQ ID NO:1, a methionine at a position corresponding to position 52 of SEQ ID NO:1, a glutamine at a position corresponding to position 69 of SEQ ID NO:1, and a lysine at a position corresponding to position 107 of SEQ ID NO:1.

In certain aspects, the modified 6C domain may comprise one or more amino acid residues selected form the group consisting of A11, A15, K22, K44, M52, Q69, and K107, of SEQ ID NO:5. In certain aspects, the modified 6C domain may comprise A11, A15, K22, K44, M52, Q69, and K107, of SEQ ID NO:5. In certain aspects, the modified 6C domain may comprise one or more amino acid residues selected from the group consisting of an alanine position 11, an alanine at position 15, a lysine at position 22, a lysine at position 44, a methionine at position 52, a glutamine at position 69, and a lysine at position 107. In certain aspects, the modified 6C domain may comprise an alanine position 11, an alanine at position 15, a lysine at position 22, a lysine at position 44, a methionine at position 52, a glutamine at position 69, and a lysine at position 107.

### Immunogen 27

In certain aspects, the modified 6C domain may comprise a mutation at one or more amino acid positions selected from the group consisting of an amino acid position corresponding to position 3 of SEQ ID NO:1, an amino acid position corresponding to position 4 of SEQ ID NO:1, an amino acid position corresponding to position 6 of SEQ ID NO:1, an amino acid position corresponding to position 11 of SEQ ID NO: 1, an amino acid position corresponding to position 14 of SEQ ID NO:1, an amino acid position corresponding to position 15 of SEQ ID NO:1, an amino acid position corresponding to position 18 of SEQ ID NO:1, an amino acid position corresponding to position 19 of SEQ ID NO:1, an amino acid position corresponding to position 21 of SEQ ID NO:1, an amino acid position corresponding to position 22 of SEQ ID NO:1, an amino acid position corresponding to position 28 of SEQ ID NO:1, an amino acid position corresponding to position 36 of SEQ ID NO:1, an amino acid position corresponding to position 43 of SEQ ID NO:1, an amino acid position corresponding to position 49 of SEQ ID NO:1, an amino acid position corresponding to position 70 of SEQ ID NO:1, an amino acid position corresponding to position 85 of SEQ ID NO:1, an amino acid position corresponding to position 89 of SEQ ID NO:1, an amino acid position corresponding to position 90 of SEQ ID NO:1, an amino acid position corresponding to position 97 of SEQ ID NO:1, an amino acid position corresponding to position 105 of SEQ ID NO:1, an amino acid position corresponding to position 107 of SEQ ID NO:1, an amino acid position corresponding to position 115 of SEQ ID NO:1, an amino acid position corresponding to position 134 of SEQ ID NO:1, and an amino acid position corresponding to position 138 of SEQ ID NO:1.

In certain aspects, the modified 6C domain may comprise one or more amino acids selected from the group consisting of a serine at a position corresponding to position 3 of SEQ ID NO:1, a threonine at a position corresponding to position 4 of SEQ ID NO:1, an arginine at a position corresponding to position 6 of SEQ ID NO: 1, an alanine at a position corresponding to position 11 of SEQ ID NO:1, a lysine at a position corresponding to position 14 of SEQ ID NO:1, an asparagine at a position corresponding to position 15 of SEQ ID NO:1, a tryptophan at a position corresponding to position 18 of SEQ ID NO:1, a glutamine at a position corresponding to position 19 of SEQ ID NO:1, an aspartic acid at a position corresponding to position 21 of SEQ ID NO: 1, an asparagine at a position corresponding to position 22 of SEQ ID NO:1, a glutamine at a position corresponding to position 28 of SEQ ID NO:1, an arginine at a position corresponding to position 36 of SEQ ID NO:1, a tyrosine at a position corresponding to position 43 of SEQ ID NO:1, an isoleucine at a position corresponding to position 49 of SEQ ID NO:1, a serine at a position corresponding to position 70 of SEQ ID NO:1, as aspartic acid at a position corresponding to position 84 of SEQ ID NO: 1, a valine at a position corresponding to position 85 of SEQ ID NO:1, an aspartic acid at a position corresponding to position 89 of SEQ ID NO:1, an asparagine at a position corresponding to position 90 of SEQ ID NO:1,an arginine at a position corresponding to position 97 of SEQ ID NO:1, a methionine at a position corresponding to position 105 of SEQ ID NO:1, a lysine at a position corresponding to position 107 of SEQ ID NO:1, a proline at a position corresponding to position 115 of SEQ ID NO:1, a lysine at a position corresponding to position 134 of SEQ ID NO:1, and a serine at a position corresponding to position 138 of SEQ ID NO:1.

In certain aspects, the modified 6C domain may comprise at least 10 amino acid residues, at least 15 amino acid residues, at least 20 amino acid residues, at least 21 amino acid residues, at least 22 amino acid residues, or at least 23 residues selected from the group consisting of S3, T4, R6, A11, K14, N15, W18, Q19, D21, N22, Q28, R36, Y43, 149, S70, V85, D89, N90, R97, M105, K107, P115, E134, and S138, of SEQ ID NO:6. In certain aspects, the modified 6C domain may comprise S3, T4, R6, A11, K14, N15, W18, Q19, D21, N22, Q28, R36, Y43, 149, S70, V85, D89, N90, R97, M105, K107, P115, E134, and S138, of SEQ ID NO:6. In certain aspects, the amino acid sequence may comprise one or more amino acids selected from the group consisting of a serine at position 3, a threonine at position 4, an arginine at position 6, an alanine at position 11, a lysine at position 14, an asparagine at position 15, a tryptophan at position 18, a glutamine at position 19, an aspartic acid at position 21, an asparagine at position 22, a glutamine at position 28, a tyrosine at position 43, an isoleucine at position 49, a serine at position 70, a valine at position 85, an aspartic acid at position 89, an asparagine at position 90, an arginine at position 97, aa methionine at position 105, lysine at position 107, a proline at position 115, a glutamic acid at position 134, and a serine at position 138. In certain aspects, the amino acid sequence may comprise a serine at position 3, a threonine at position 4, an arginine at position 6, an alanine at position 11, a lysine at position 14, an asparagine at position 15, a tryptophan at position 18, a glutamine at position 19, an aspartic acid at position 21, an asparagine at position 22, a glutamine at position 28, a tyrosine at position 43, an isoleucine at position 49, a serine at position 70, a valine at position 85, an aspartic acid at position 89, an asparagine at position 90, an arginine at position 97, aa methionine at position 105, lysine at position 107, a proline at position 115, a glutamic acid at position 134, and a serine at position 138. In certain aspects, the amino acid sequence may comprise SEQ ID NO:6.

### Immunogen 15-1.5

In certain aspects, the modified 6C domain may comprise a mutation at one or more amino acid positions selected from the group consisting of an amino acid position corresponding to position 6 of SEQ ID NO:1, an amino acid position corresponding to position 12 of SEQ ID NO:1, an amino acid position corresponding to position 14 of SEQ ID NO:1, an amino acid position corresponding to position 15 of SEQ ID NO:1, an amino acid position corresponding to position 18 of SEQ ID NO:1, an amino acid position corresponding to position 19 of SEQ ID NO:1, an amino acid position corresponding to position 21 of SEQ ID NO:1, an amino acid position corresponding to position 22 of SEQ ID NO:1, an amino acid position corresponding to position 28 of SEQ ID NO:1, an amino acid position corresponding to position 33 of SEQ ID NO:1, an amino acid position corresponding to position 36 of SEQ ID NO:1, an amino acid position corresponding to position 38 of SEQ ID NO:1, an amino acid position corresponding to position 43 of SEQ ID NO:1, an amino acid position corresponding to position 49 of SEQ ID NO:1, an amino acid position corresponding to position 69 of SEQ ID NO:1, an amino acid position corresponding to position 70 of SEQ ID NO:1, an amino acid position corresponding to position 71 of SEQ ID NO:1, an amino acid position corresponding to position 83 of SEQ ID NO:1, an amino acid position corresponding to position 86 of SEQ ID NO:1, an amino acid position corresponding to position 89 of SEQ ID NO:1, an amino acid position corresponding to position 97 of SEQ ID NO:1, an amino acid position corresponding to position 105 of SEQ ID NO:1, an amino acid position corresponding to position 107 of SEQ ID NO:1, an amino acid position corresponding to position 115 of SEQ ID NO:1, and an amino acid position corresponding to position 134 of SEQ ID NO:1.

In certain aspects, the modified 6C domain may comprise one or more amino acids selected from the group consisting of an alanine at a position corresponding to position 6 of SEQ ID NO:1, an alanine at a position corresponding to position 12 of SEQ ID NO:1, a lysine at a position corresponding to position 14 of SEQ ID NO: 1, an alanine at a position corresponding to position 15 of SEQ ID NO:1, a tyrosine at a position corresponding to position 18 of SEQ ID NO:1, a lysine at a position corresponding to position 19 of SEQ ID NO:1, an alanine at a position corresponding to position 21 of SEQ ID NO:1, a lysine at a position corresponding to position 22 of SEQ ID NO: 1, a glutamine at a position corresponding to position 28 of SEQ ID NO:1, a lysine at a position corresponding to position 33 of SEQ ID NO:1, an arginine at a position corresponding to position 36 of SEQ ID NO:1, a glutamic acid at a position corresponding to position 38 of SEQ ID NO:1, a histidine at a position corresponding to position 43 of SEQ ID NO:1, an isoleucine at a position corresponding to position 49 of SEQ ID NO:1, an asparagine at a position corresponding to position 69 of SEQ ID NO:1, a serine at a position corresponding to position 70 of SEQ ID NO:1, an alanine at a position corresponding to position 71 of SEQ ID NO:1, glutamic acid at a position corresponding to position 83 of SEQ ID NO:1, a leucine at a position corresponding to position 86 of SEQ ID NO:1, an alanine at a position corresponding to position 89 of SEQ ID NO:1, an arginine at a position corresponding to position 97 of SEQ ID NO:1, a methionine at a position corresponding to position 105 of SEQ ID NO:1, an alanine at a position corresponding to position 107 of SEQ ID NO:1, a proline at a position corresponding to position 115 of SEQ ID NO:1, and a phenylalanine at a position corresponding to position 134 of SEQ ID NO:1.

In certain aspects, the modified 6C domain may comprise at least 10 amino acid residues, at least 15 amino acid residues, at least 20 amino acid residues, at least 21 amino acid residues, at least 22 amino acid residues, at least 23 amino acid residues, or at least 24 amino acid residues selected from the group consisting of A6, A12, K14, A15, Y18, K19, A21, K22, Q28, K33, R36, E38, H43, 149, N69, S70, A71, E83, L86, A89, R97, M105, A107, P115, and F134, of SEQ ID NO:7. In certain aspects, the modified 6C domain may comprise A6, A12, K14, A15, Y18, K19, A21, K22, Q28, K33, R36, E38, H43, 149, N69, S70, A71, E83, L86, A89, R97, M105, A107, P115, and F134, of SEQ ID NO:7. In certain aspects, the amino acid sequence may comprise one or more amino acids selected from the group consisting of an alanine at position 6, an alanine at position 12, a lysine at position 14, an alanine at position 15, a tyrosine at position 18, a lysine at position 19, an alanine at position 21, a lysine at position 22, a glutamine at position 28, a lysine at position 33, an arginine at position 36, a glutamic acid at position 38, a histidine at position 43, an isoleucine at position 49, an asparagine at position 69, a serine at position 70, an alanine at position 71, a glutamic acid at position 83, a leucine at position 86, an alanine at position 89, an arginine at position 97, a methionine at position 105, lysine at position 107, a proline at position 115, and a phenylalanine at position 134. In certain aspects, the amino acid sequence may comprise an alanine at position 6, an alanine at position 12, a lysine at position 14, an alanine at position 15, a tyrosine at position 18, a lysine at position 19, an alanine at position 21, a lysine at position 22, a glutamine at position 28, a lysine at position 33, an arginine at position 36, a glutamic acid at position 38, a histidine at position 43, an isoleucine at position 49, an asparagine at position 69, a serine at position 70, an alanine at position 71, a glutamic acid at position 83, a leucine at position 86, an alanine at position 89, an arginine at position 97, a methionine at position 105, lysine at position 107, a proline at position 115, and a phenylalanine at position 134. In certain aspects, the amino acid sequence may comprise SEQ ID NO:7.

### Immunogen 15-3

In certain aspects, the modified 6C domain may comprise a mutation at one or more amino acid positions selected from the group consisting of an amino acid position corresponding to position 6 of SEQ ID NO:1, an amino acid position corresponding to position 11 of SEQ ID NO:1, an amino acid position corresponding to position 15 of SEQ ID NO:1, an amino acid position corresponding to position 38 of SEQ ID NO:1, an amino acid position corresponding to position 69 of SEQ ID NO:1, an amino acid position corresponding to position 70 of SEQ ID NO:1, an amino acid position corresponding to position 71 of SEQ ID NO:1, an amino acid position corresponding to position 83 of SEQ ID NO:1,an amino acid position corresponding to position 89 of SEQ ID NO:1, an amino acid position corresponding to position 97 of SEQ ID NO:1, an amino acid position corresponding to position 105 of SEQ ID NO:1, an amino acid position corresponding to position 107 of SEQ ID NO:1, an amino acid position corresponding to position 115 of SEQ ID NO:1, and an amino acid position corresponding to position 134 of SEQ ID NO:1.

In certain aspects, the modified 6C domain may comprise one or more amino acids selected from the group consisting of an alanine at a position corresponding to position 6 of SEQ ID NO:1, an alanine at a position corresponding to position 11 of SEQ ID NO:1, an alanine at a position corresponding to position 15 of SEQ ID NO:1, a lysine at a position corresponding to position 22 of SEQ ID NO:1, a glutamic acid at a position corresponding to position 38 of SEQ ID NO:1, an asparagine at a position corresponding to position 69 of SEQ ID NO:1, a serine at a position corresponding to position 70 of SEQ ID NO:1, an alanine at a position corresponding to position 71 of SEQ ID NO:1, glutamic acid at a position corresponding to position 83 of SEQ ID NO:1, an alanine at a position corresponding to position 89 of SEQ ID NO:1, an arginine at a position corresponding to position 97 of SEQ ID NO:1, a methionine at a position corresponding to position 105 of SEQ ID NO:1, an alanine at a position corresponding to position 107 of SEQ ID NO:1, a proline at a position corresponding to position 115 of SEQ ID NO:1, and a phenylalanine at a position corresponding to position 134 of SEQ ID NO:1.

In certain aspects, the modified 6C domain may comprise at least 10 amino acid residues, at least 10 amino acid residues, at least 12 amino acid residues, at least 13 amino acid residues, or at least 15 amino acid residues selected from the group consisting of A6, A11, A15, K22, E38, N69, S70, A71, E83, A89, R97, M105, A107, P115, and F134, of SEQ ID NO:8. In certain aspects, the modified 6C domain may comprise A6, A11, A15K22, E38, N69, S70, A71, E83, A89, R97, M105, A107, P115, and F134, of SEQ ID NO:8. In certain aspects, the amino acid sequence may comprise one or more amino acids selected from the group consisting of an alanine at position 6, an alanine at position 11, an alanine at position 15, a lysine at position 22, a glutamic acid at position 38, an asparagine at position 69, a serine at position 70, an alanine at position 71, a glutamic acid at position 83, an alanine at position 89, an arginine at position 97, a methionine at position 105, an alanine at position 107, a proline at position 115, and a phenylalanine at position 134. In certain aspects, the amino acid sequence may comprise an alanine at position 6, an alanine at position 11, an alanine at position 15, a lysine at position 22, a glutamic acid at position 38, an asparagine at position 69, a serine at position 70, an alanine at position 71, a glutamic acid at position 83, an alanine at position 89, an arginine at position 97, a methionine at position 105, an alanine at position 107, a proline at position 115, and a phenylalanine at position 134. In certain aspects, the amino acid sequence may comprise SEQ ID NO:8.

### Immunogen 15-4

In certain aspects, the modified 6C domain may comprise a mutation at one or more amino acid positions selected from the group consisting of an amino acid position corresponding to position 11 of SEQ ID NO:1, an amino acid position corresponding to position 15 of SEQ ID NO:1, an amino acid position corresponding to position 38 of SEQ ID NO:1, an amino acid position corresponding to position 69 of SEQ ID NO:1, an amino acid position corresponding to position 70 of SEQ ID NO:1, an amino acid position corresponding to position 71 of SEQ ID NO:1, an amino acid position corresponding to position 83 of SEQ ID NO:1, an amino acid position corresponding to position 86 of SEQ ID NO:1, an amino acid position corresponding to position 105 of SEQ ID NO:1, an amino acid position corresponding to position 107 of SEQ ID NO:1, and an amino acid position corresponding to position 115 of SEQ ID NO:1.

In certain aspects, the modified 6C domain may comprise one or more amino acids selected from the group consisting of an alanine at a position corresponding to position 11 of SEQ ID NO:1, an alanine at a position corresponding to position 15 of SEQ ID NO:1, a glutamic acid at a position corresponding to position 38 of SEQ ID NO:1, an asparagine at a position corresponding to position 69 of SEQ ID NO: 1, a serine at a position corresponding to position 70 of SEQ ID NO:1, an alanine at a position corresponding to position 71 of SEQ ID NO:1, glutamic acid at a position corresponding to position 83 of SEQ ID NO:1, a leucine at a position corresponding to position 86 of SEQ ID NO:1, an arginine at a position corresponding to position 97 of SEQ ID NO:1, a methionine at a position corresponding to position 105 of SEQ ID NO: 1, an alanine at a position corresponding to position 107 of SEQ ID NO:1, and a proline at a position corresponding to position 115 of SEQ ID NO:1.

In certain aspects, the modified 6C domain may comprise at least 10 amino acid residues, at least 8 amino acid residues, at least 9 amino acid residues, or at least 10 amino acid residues selected from the group consisting of A11, A15, E38, N69, S70, A71, E83, L86, M105, A107, and P115, of SEQ ID NO:9. In certain aspects, the modified 6C domain may comprise A11, A15, E38, N69, S70, A71, E83, L86, M105, A107, and P115, of SEQ ID NO:9. In certain aspects, the amino acid sequence may comprise one or more amino acids selected from the group consisting of an alanine at position 11, an alanine at position 15, a glutamic acid at position 38, an asparagine at position 69, a serine at position 70, an alanine at position 71, a glutamic acid at position 83, a leucine at position 86, a methionine at position 105, an alanine at position 107, and a proline at position 115. In certain aspects, the amino acid sequence may comprise an alanine at position 11, an alanine at position 15, a glutamic acid at position 38, an asparagine at position 69, a serine at position 70, an alanine at position 71, a glutamic acid at position 83, a leucine at position 86, a methionine at position 105, an alanine at position 107, and a proline at position 115. In certain aspects, the amino acid sequence may comprise SEQ ID NO:9.

### Immunogen 15-5

In certain aspects, the modified 6C domain may comprise a mutation at one or more amino acid positions selected from the group consisting of an amino acid position corresponding to position 11 of SEQ ID NO:1, an amino acid position corresponding to position 15 of SEQ ID NO:1, an amino acid position corresponding to position 38 of SEQ ID NO:1, an amino acid position corresponding to position 69 of SEQ ID NO:1, an amino acid position corresponding to position 71 of SEQ ID NO:1, an amino acid position corresponding to position 86 of SEQ ID NO:1, an amino acid position corresponding to position 105 of SEQ ID NO:1, and an amino acid position corresponding to position 107 of SEQ ID NO:1.

In certain aspects, the modified 6C domain may comprise one or more amino acids selected from the group consisting of an alanine at a position corresponding to position 11 of SEQ ID NO:1, an alanine at a position corresponding to position 15 of SEQ ID NO:1, a glutamic acid at a position corresponding to position 38 of SEQ ID NO:1, an asparagine at a position corresponding to position 69 of SEQ ID NO: 1, an alanine at a position corresponding to position 71 of SEQ ID NO:1, a leucine at a position corresponding to position 86 of SEQ ID NO:1, an arginine at a position corresponding to position 97 of SEQ ID NO:1, a methionine at a position corresponding to position 105 of SEQ ID NO:1, and an alanine at a position corresponding to position 107 of SEQ ID NO:1.

In certain aspects, the modified 6C domain may comprise at least 10 amino acid residues, at least 4 amino acid residues, at least 5 amino acid residues, at least 6 amino acid residues, or at least 7 amino acid residues selected from the group consisting of A11, A15, E38, N69, A71, L86, M105, and A107, of SEQ ID NO: 10. In certain aspects, the modified 6C domain may comprise A11, A15, E38, N69, A71, L86, M105, A107, of SEQ ID NO:10. In certain aspects, the amino acid sequence may comprise one or more amino acids selected from the group consisting of an alanine at position 11, an alanine at position 15, a glutamic acid at position 38, an asparagine at position 69, an alanine at position 71, a leucine at position 86, a methionine at position 105, and an alanine at position 107. In certain aspects, the amino acid sequence may comprise an alanine at position 11, an alanine at position 15, a glutamic acid at position 38, an asparagine at position 69, an alanine at position 71, a leucine at position 86, a methionine at position 105, and an alanine at position 107. In certain aspects, the amino acid sequence may comprise SEQ ID NO:10.

### Immunogen 15-6

In certain aspects, the modified 6C domain may comprise a mutation at one or more amino acid positions selected from the group consisting of an amino acid position corresponding to position 11 of SEQ ID NO:1, an amino acid position corresponding to position 15 of SEQ ID NO:1, an amino acid position corresponding to position 69 of SEQ ID NO:1, an amino acid position corresponding to position 71 of SEQ ID NO:1, and an amino acid position corresponding to position 105 of SEQ ID NO:1.

In certain aspects, the modified 6C domain may comprise one or more amino acids selected from the group consisting of an alanine at a position corresponding to position 11 of SEQ ID NO:1, an alanine at a position corresponding to position 15 of SEQ ID NO:1, an asparagine at a position corresponding to position 69 of SEQ ID NO:1, an alanine at a position corresponding to position 71 of SEQ ID NO:1, and a methionine at a position corresponding to position 105 of SEQ ID NO:1.

In certain aspects, the modified 6C domain may comprise at least 10 amino acid residues, at least 3 amino acid residues, or at least 4 amino acid residues selected from the group consisting of A11, A15, N69, A71, and M105, of SEQ ID NO: 11. In certain aspects, the modified 6C domain may comprise A11, A15, N69, A71, and M105, of SEQ ID NO:11. In certain aspects, the amino acid sequence may comprise one or more amino acids selected from the group consisting of an alanine at position 11, an alanine at position 15, an asparagine at position 69, an alanine at position 71, and a methionine at position 105. In certain aspects, the amino acid sequence may comprise an alanine at position 11, an alanine at position 15, an asparagine at position 69, an alanine at position 71, and a methionine at position 105. In certain aspects, the amino acid sequence may comprise SEQ ID NO:11.

### Immunogen 15-7

In certain aspects, the modified 6C domain may comprise a mutation at one or more amino acid positions selected from the group consisting of an amino acid position corresponding to position 11 of SEQ ID NO:1, an amino acid position corresponding to position 15 of SEQ ID NO:1, an amino acid position corresponding to position 69 of SEQ ID NO:1, and an amino acid position corresponding to position 71 of SEQ ID NO:1.

In certain aspects, the modified 6C domain may comprise one or more amino acids selected from the group consisting of an alanine at a position corresponding to position 11 of SEQ ID NO:1, an alanine at a position corresponding to position 15 of SEQ ID NO:1, an asparagine at a position corresponding to position 69 of SEQ ID NO:1, and an alanine at a position corresponding to position 71 of SEQ ID NO: 1.

In certain aspects, the modified 6C domain may comprise at least 10 amino acid residues, at least 2 amino acid residues, or at least 3 amino acid residues selected from the group consisting of A11, A15, N69, and A71, of SEQ ID NO: 11. In certain aspects, the modified 6C domain may comprise A11, A15, N69, and A71, of SEQ ID NO:12. In certain aspects, the amino acid sequence may comprise one or more amino acids selected from the group consisting of an alanine at position 11, an alanine at position 15, an asparagine at position 69, and an alanine at position 71. In certain aspects, the amino acid sequence may comprise an alanine at position 11, an alanine at position 15, an asparagine at position 69, and an alanine at position 71. In certain aspects, the amino acid sequence may comprise SEQ ID NO:12.

### Immunogen 17-1.5

In certain aspects, the modified 6C domain may comprise a mutation at one or more amino acid positions selected from the group consisting of an amino acid position corresponding to position 11 of SEQ ID NO:1, an amino acid position corresponding to position 14 of SEQ ID NO:1, an amino acid position corresponding to position 15 of SEQ ID NO:1, an amino acid position corresponding to position 16 of SEQ ID NO:1, an amino acid position corresponding to position 19 of SEQ ID NO:1, an amino acid position corresponding to position 21 of SEQ ID NO:1, an amino acid position corresponding to position 22 of SEQ ID NO:1, an amino acid position corresponding to position 28 of SEQ ID NO:1, an amino acid position corresponding to position 33 of SEQ ID NO:1, an amino acid position corresponding to position 36 of SEQ ID NO:1, an amino acid position corresponding to position 38 of SEQ ID NO:1, an amino acid position corresponding to position 40 of SEQ ID NO:1, an amino acid position corresponding to position 43 of SEQ ID NO:1, an amino acid position corresponding to position 44 of SEQ ID NO:1, an amino acid position corresponding to position 49 of SEQ ID NO:1, an amino acid position corresponding to position 52 of SEQ ID NO:1, an amino acid position corresponding to position 69 of SEQ ID NO:1, an amino acid position corresponding to position 70 of SEQ ID NO:1, an amino acid position corresponding to position 85 of SEQ ID NO:1, an amino acid position corresponding to position 89 of SEQ ID NO:1, an amino acid position corresponding to position 97 of SEQ ID NO:1, an amino acid position corresponding to position 107 of SEQ ID NO:1, and an amino acid position corresponding to position 115 of SEQ ID NO:1.

In certain aspects, the modified 6C domain may comprise one or more amino acids selected from the group consisting of an alanine at a position corresponding to position 11 of SEQ ID NO:1, a lysine at a position corresponding to position 14 of SEQ ID NO:1, an alanine at a position corresponding to position 15 of SEQ ID NO:1, a serine at a position corresponding to position 16 of SEQ ID NO:1, a lysine at a position corresponding to position 19 of SEQ ID NO:1, an alanine at a position corresponding to position 21 of SEQ ID NO:1, a lysine at a position corresponding to position 22 of SEQ ID NO:1, a glutamine at a position corresponding to position 28 of SEQ ID NO:1, a lysine at a position corresponding to position 33 of SEQ ID NO: 1, an arginine at a position corresponding to position 36 of SEQ ID NO:1, a glutamic acid at a position corresponding to position 38 of SEQ ID NO:1, a glutamic acid at a position corresponding to position 40 of SEQ ID NO:1, a histidine at a position corresponding to position 43 of SEQ ID NO:1, a lysine at a position corresponding to position 44 of SEQ ID NO:1, an isoleucine at a position corresponding to position 49 of SEQ ID NO:1, a methionine at a position corresponding to position 52 of SEQ ID NO:1, a glutamine at a position corresponding to position 69 of SEQ ID NO:1, a serine at a position corresponding to position 70 of SEQ ID NO:1, a valine at a position corresponding to position 85 of SEQ ID NO:1, an aspartic acid at a position corresponding to position 89 of SEQ ID NO:1, an arginine at a position corresponding to position 97 of SEQ ID NO:1, a lysine at a position corresponding to position 107 of SEQ ID NO: 1, and a proline at a position corresponding to position 115 of SEQ ID NO:1.

In certain aspects, the modified 6C domain may comprise at least 10 amino acid residues, at least 18 amino acid residues, at least 20 amino acid residues, at least 21 amino acid residues, or at least 22 amino acid residues, selected from the group consisting of A11, K14, A15, S16, K19, A21, K22, Q28, K33, R36, E38, E40, H43, K44, 149, M52, Q69, S70, V85, D89, R97, K107, and P115, of SEQ ID NO: 13. In certain aspects, the modified 6C domain may comprise A11, K14, A15, S16, K19, A21, K22, Q28, K33, R36, E38, E40, H43, K44, 149, M52, Q69, S70, V85, D89, R97, K107, and P115, of SEQ ID NO:13. In certain aspects, the amino acid sequence may comprise one or more amino acids selected from the group consisting of an alanine at position 11, a lysine at position 14, an alanine at position 15, serine at position 16, a lysine at position 19, an alanine at position 21, a lysine at position 22, a glutamine at position 28, a lysine at position 33, an arginine at position 36, a glutamic acid at position 38, a glutamic acid at position 40, a histidine at position 43, a lysine at position 44, an isoleucine at position 49, a methionine at position 52, a glutamine at position 69, a serine at position 70, a valine at position 85, an aspartic acid at position 89, an arginine at position 97, a lysine at position 107, and a proline at position 115. In certain aspects, the amino acid sequence may comprise an alanine at position 11, a lysine at position 14, an alanine at position 15, serine at position 16, a lysine at position 19, an alanine at position 21, a lysine at position 22, a glutamine at position 28, a lysine at position 33, an arginine at position 36, a glutamic acid at position 38, a glutamic acid at position 40, a histidine at position 43, a lysine at position 44, an isoleucine at position 49, a methionine at position 52, a glutamine at position 69, a serine at position 70, a valine at position 85, an aspartic acid at position 89, an arginine at position 97, a lysine at position 107, and a proline at position 115. In certain aspects, the amino acid sequence may comprise SEQ ID NO:13.

### Immunogen 17-2

In certain aspects, the modified 6C domain may comprise a mutation at one or more amino acid positions selected from the group consisting of an amino acid position corresponding to position 11 of SEQ ID NO:1, an amino acid position corresponding to position 14 of SEQ ID NO:1, an amino acid position corresponding to position 15 of SEQ ID NO:1, an amino acid position corresponding to position 19 of SEQ ID NO:1, an amino acid position corresponding to position 22 of SEQ ID NO:1, an amino acid position corresponding to position 33 of SEQ ID NO:1, an amino acid position corresponding to position 36 of SEQ ID NO:1, an amino acid position corresponding to position 38 of SEQ ID NO:1, an amino acid position corresponding to position 40 of SEQ ID NO:1, an amino acid position corresponding to position 43 of SEQ ID NO:1, an amino acid position corresponding to position 44 of SEQ ID NO:1, an amino acid position corresponding to position 69 of SEQ ID NO:1, an amino acid position corresponding to position 70 of SEQ ID NO:1, an amino acid position corresponding to position 85 of SEQ ID NO:1, an amino acid position corresponding to position 89 of SEQ ID NO:1, an amino acid position corresponding to position 97 of SEQ ID NO:1, an amino acid position corresponding to position 107 of SEQ ID NO:1, and an amino acid position corresponding to position 115 of SEQ ID NO: 1.

In certain aspects, the modified 6C domain may comprise one or more amino acids selected from the group consisting of an alanine at a position corresponding to position 11 of SEQ ID NO:1, a lysine at a position corresponding to position 14 of SEQ ID NO:1, an alanine at a position corresponding to position 15 of SEQ ID NO:1, a lysine at a position corresponding to position 19 of SEQ ID NO:1, a lysine at a position corresponding to position 22 of SEQ ID NO:1, a lysine at a position corresponding to position 33 of SEQ ID NO:1, an arginine at a position corresponding to position 36 of SEQ ID NO:1, a glutamic acid at a position corresponding to position 38 of SEQ ID NO:1, a glutamic acid at a position corresponding to position 40 of SEQ ID NO: 1, a histidine at a position corresponding to position 43 of SEQ ID NO:1, a lysine at a position corresponding to position 44 of SEQ ID NO:1, a glutamine at a position corresponding to position 69 of SEQ ID NO:1, a serine at a position corresponding to position 70 of SEQ ID NO:1, a valine at a position corresponding to position 85 of SEQ ID NO: 1, an aspartic acid at a position corresponding to position 89 of SEQ ID NO:1, an arginine at a position corresponding to position 97 of SEQ ID NO:1, a lysine at a position corresponding to position 107 of SEQ ID NO:1, and a proline at a position corresponding to position 115 of SEQ ID NO:1.

In certain aspects, the modified 6C domain may comprise at least 10 amino acid residues, at least 15 amino acid residues, at least 16 amino acid residues, or at least 17 amino acid residues selected from the group consisting of A11, K14, A15, K19, K22, K33, R36, E38, E40, H43, K44, Q69, S70, V85, D89, R97, K107, and P115, of SEQ ID NO:14. In certain aspects, the modified 6C domain may comprise A11, K14, A15, K19, K22, K33, R36, E38, E40, H43, K44, Q69, S70, V85, D89, R97, K107, and P115, of SEQ ID NO:14. In certain aspects, the amino acid sequence may comprise one or more amino acids selected from the group consisting of an alanine at position 11, a lysine at position 14, an alanine at position 15, a lysine at position 19, a lysine at position 22, a lysine at position 33, an arginine at position 36, a glutamic acid at position 38, a glutamic acid at position 40, a histidine at position 43, a lysine at position 44, a glutamine at position 69, a serine at position 70, a valine at position 85, an aspartic acid at position 89, an arginine at position 97, a lysine at position 107, and a proline at position 115. In certain aspects, the amino acid sequence may comprise an alanine at position 11, a lysine at position 14, an alanine at position 15, a lysine at position 19, a lysine at position 22, a lysine at position 33, an arginine at position 36, a glutamic acid at position 38, a glutamic acid at position 40, a histidine at position 43, a lysine at position 44, a glutamine at position 69, a serine at position 70, a valine at position 85, an aspartic acid at position 89, an arginine at position 97, a lysine at position 107, and a proline at position 115. In certain aspects, the amino acid sequence may comprise SEQ ID NO:14.

### Immunogen 17-3

In certain aspects, the modified 6C domain may comprise a mutation at one or more amino acid positions selected from the group consisting of an amino acid position corresponding to position 11 of SEQ ID NO:1, an amino acid position corresponding to position 15 of SEQ ID NO:1, an amino acid position corresponding to position 22 of SEQ ID NO:1, an amino acid position corresponding to position 38 of SEQ ID NO:1, an amino acid position corresponding to position 43 of SEQ ID NO:1, an amino acid position corresponding to position 44 of SEQ ID NO:1, an amino acid position corresponding to position 49 of SEQ ID NO:1, an amino acid position corresponding to position 52 of SEQ ID NO:1, an amino acid position corresponding to position 69 of SEQ ID NO:1, an amino acid position corresponding to position 70 of SEQ ID NO:1, an amino acid position corresponding to position 85 of SEQ ID NO:1, an amino acid position corresponding to position 89 of SEQ ID NO:1, an amino acid position corresponding to position 97 of SEQ ID NO:1, and an amino acid position corresponding to position 107 of SEQ ID NO:1.

In certain aspects, the modified 6C domain may comprise one or more amino acids selected from the group consisting of an alanine at a position corresponding to position 11 of SEQ ID NO:1, an alanine at a position corresponding to position 15 of SEQ ID NO:1, a lysine at a position corresponding to position 22 of SEQ ID NO: 1, a glutamic acid at a position corresponding to position 38 of SEQ ID NO:1, a histidine at a position corresponding to position 43 of SEQ ID NO:1, a lysine at a position corresponding to position 44 of SEQ ID NO:1, an isoleucine at apposition corresponding to position 49 of SEQ ID NO:1, a methionine at apposition corresponding to position 52 of SEQ ID NO:1, a glutamine at a position corresponding to position 69 of SEQ ID NO:1, a serine at a position corresponding to position 70 of SEQ ID NO:1, a valine at a position corresponding to position 85 of SEQ ID NO:1, an aspartic acid at a position corresponding to position 89 of SEQ ID NO:1, an arginine at a position corresponding to position 97 of SEQ ID NO:1, and a lysine at a position corresponding to position 107 of SEQ ID NO:1.

In certain aspects, the modified 6C domain may comprise at least 10 amino acid residues, at least 10 amino acid residues, at least 11 amino acid residues, at least 12 amino acid residues, or at least 13 amino acid residues selected from the group consisting of A11, A15, K22, E38, H43, K44, 149, M52, Q69, S70, V85, D89, R97, and K107, of SEQ ID NO:15. In certain aspects, the modified 6C domain may comprise A11, A15, K22, E38, H43, K44, 149, M52, Q69, S70, V85, D89, R97, and K107, of SEQ ID NO:15. In certain aspects, the amino acid sequence may comprise one or more amino acids selected from the group consisting of an alanine at position 11, an alanine at position 15, a lysine at position 22, a glutamic acid at position 38, a histidine at position 43, a lysine at position 44, an isoleucine at position 49, a methionine at position 52, a glutamine at position 69, a serine at position 70, a valine at position 85, an aspartic acid at position 89, an arginine at position 97, and a lysine at position 107. In certain aspects, the amino acid sequence may comprise an alanine at position 11, an alanine at position 15, a lysine at position 22, a glutamic acid at position 38, a histidine at position 43, a lysine at position 44, an isoleucine at position 49, a methionine at position 52, a glutamine at position 69, a serine at position 70, a valine at position 85, an aspartic acid at position 89, an arginine at position 97, and a lysine at position 107. In certain aspects, the amino acid sequence may comprise SEQ ID NO:15.

### Immunogen 17-5

In certain aspects, the modified 6C domain may comprise a mutation at one or more amino acid positions selected from the group consisting of an amino acid position corresponding to position 11 of SEQ ID NO:1, an amino acid position corresponding to position 15 of SEQ ID NO:1, an amino acid position corresponding to position 44 of SEQ ID NO:1, an amino acid position corresponding to position 52 of SEQ ID NO:1, an amino acid position corresponding to position 69 of SEQ ID NO:1, and an amino acid position corresponding to position 107 of SEQ ID NO: 1.

In certain aspects, the modified 6C domain may comprise one or more amino acids selected from the group consisting of an alanine at a position corresponding to position 11 of SEQ ID NO:1, an alanine at a position corresponding to position 15 of SEQ ID NO:1, a lysine at a position corresponding to position 44 of SEQ ID NO: 1, a methionine at apposition corresponding to position 52 of SEQ ID NO:1, a glutamine at a position corresponding to position 69 of SEQ ID NO:1, and a lysine at a position corresponding to position 107 of SEQ ID NO:1.

In certain aspects, the modified 6C domain may comprise at least 10 amino acid residues, at least 3 amino acid residues, at least 4 amino acid residues, or at least 5 amino acid residues, selected from the group consisting of A11, A15, K44, M52, Q69, and K107, of SEQ ID NO:16. In certain aspects, the modified 6C domain may comprise A11, A15, K44, M52, Q69, and K107, of SEQ ID NO: 16. In certain aspects, the amino acid sequence may comprise one or more amino acids selected from the group consisting of an alanine at position 11, an alanine at position 15, a lysine at position 44, a methionine at position 52, a glutamine at position 69, and a lysine at position 107. In certain aspects, the amino acid sequence may comprise an alanine at position 11, an alanine at position 15, a lysine at position 44, a methionine at position 52, a glutamine at position 69, and a lysine at position 107. In certain aspects, the amino acid sequence may comprise SEQ ID NO:16.

### Immunogen 17-6

In certain aspects, the modified 6C domain may comprise a mutation at one or more amino acid positions selected from the group consisting of an amino acid position corresponding to position 11 of SEQ ID NO:1, an amino acid position corresponding to position 15 of SEQ ID NO:1, an amino acid position corresponding to position 52 of SEQ ID NO:1, and an amino acid position corresponding to position 107 of SEQ ID NO:1.

In certain aspects, the modified 6C domain may comprise one or more amino acids selected from the group consisting of an alanine at a position corresponding to position 11 of SEQ ID NO:1, an alanine at a position corresponding to position 15 of SEQ ID NO:1, a methionine at apposition corresponding to position 52 of SEQ ID NO:1, and a lysine at a position corresponding to position 107 of SEQ ID NO:1.

In certain aspects, the modified 6C domain may comprise at least 10 amino acid residues, at least 2 amino acid residues, or at least 3 amino acid residues, selected from the group consisting of A11, A15, M52, and K107, of SEQ ID NO: 17. In certain aspects, the modified 6C domain may comprise A11, A15, M52, and K107, of SEQ ID NO:17. In certain aspects, the amino acid sequence may comprise one or more amino acids selected from the group consisting of an alanine at position 11, an alanine at position 15, a methionine at position 52, and a lysine at position 107. In certain aspects, the amino acid sequence may comprise SEQ ID NO:17.

In certain aspects, a modified 6C domain of the disclosure lacks an N-glycosylation signal and therefore, when expressed, is not glycosylated. In certain aspects, the modified 6C domain lacks an N-glycosylation signal due to mutation of a naturally occurring N-glycosylation signal in the sequence of the 6C domain. In certain aspects, one or more amino acid position corresponding amino acid positions 9-11 of SEQ ID NO:1 may comprise a mutation relative to SEQ ID NO:1. In certain aspects, one or more amino acid position corresponding amino acid positions 13-15 of SEQ ID NO:1 may comprise a mutation relative to SEQ ID NO:1.

Another protein expressed by Plasmodium spp. that may be used as an immunogen is the circumsporozoite protein (CSP). CSP is the dominant protein expressed on the surface of Plasmodium sporozoites and it plays a crucial role in the invasion by sporozoites of hepatocytes. The CSP consists of a conserved N-terminal region with a charged protease-cleavage site known as region I, a central diverse repeat region of 25-49 NANP repeats, and a conserved C-terminal region comprising a short, conserved region III and a thrombospondin-like type 1 repeat (TSR) domain. The CSP also contains a junctional region that joins the N-terminal region to the central diverse repeat region. An exemplary CSP is shown in FIG. 15, which also illustrates the relationship between the various regions. In certain aspects, an immunogen of the disclosure may comprise a modified CSP protein, wherein the modified CSP protein comprises a CSP junction region, or a portion thereof, preferably comprising the nAB CIS43 epitope (e.g., SEQ ID NO:37), joined to a NANP (SEQ ID NO:39) repeat region, the repeat region comprising a plurality of consecutive NANP amino acid sequences, wherein NANP repeat region is joined to a portion of the conserved C-terminal region, and wherein the modified CSP lacks the N-terminal region of a wild-type (wt) CSP protein. In certain aspects, the carboxy terminal end of the junction region, or a portion thereof, is joined to the amino terminal end of the NANP repeat region. In certain aspects the carboxy terminal end of the NANP repeat region is joined to the amino terminal end of the portion of the conserved C-terminal region. In certain aspects, the junction region, or a portion thereof, preferably comprising the nAB CIS43 epitope, comprises an amino acid sequence corresponding to amino acids 120-131 of SEQ ID NO:31. In certain asepcts, the junction region, or a portion thereof, used to produce a modified CSP may comprise, consist of, consist essentially of, amino acids 120-131 of SEQ ID NO:31. In certain aspects, the junction region comprises, or a portion thereof, consists of, or consists essentially of, amino acid sequence at least 83%, or 100%, identical to SEQ ID NO: 37. In certain aspects, the NANP repeat region comprises between three and seven consecutive NANP amino acid sequences. In certain aspects, the NANP repeat region comprises between four and six consecutive NANP amino acid sequences. In certain aspects, the NANP repeat region comprises five consecutive NANP amino acid sequences. In certain aspects, the portion of the conserved-C terminal region comprises one or more T-cell epitopes present in the wt CSP protein. In certain aspects, the portion of the conserved-C terminal region comprises a thrombospondin-like type 1 repeat (TSR) domain. In certain aspects, the portion of the conserved-C terminal region may comprise an amino acid sequence corresponding to amino acids 313-386 of SEQ ID NO:31. In certain aspects, the conserved-C terminal region may comprise, consist of, or consist essentially of, amino acid sequence at least 90% %, at least 95%, at least 97%, or 100%, identical to SEQ ID NO:38. In certain aspects, the modified CSP lacks an amino acid sequence corresponding to amino acids 1-119, or 18-119, of SEQ ID NO:31. In certain aspects, the junction region, or a portion thereof, preferably comprising the nAB CIS43 epitope, may be joined directly to NANP repeat region, or the two regions may be joined by a linker. In certain aspects, the NANP repeat region may be joined directly to the portion of the conserved C-terminal region, or the two regions may be joined by a linker.

In certain aspects, a modified CSP of the disclosure comprises, consists of, or consists essentially of, a junction region, or a portion thereof (e.g., SEQ ID NO: 37), joined a NANP repeat region, which is joined to a conserved C-terminal region, wherein the junction region, or a portion thereof, preferably comprising the nAB CIS43 epitope, (e.g., SEQ ID NO:37), comprises, consists of, or consist essentially of, an amino acid sequence at least 83%, or 100%, identical to SEQ ID NO:37, wherein the NANP repeat region consists, or consists essentially of, three, four, five, six, or seven consecutive NANP amino acid sequences, and wherein the conserved-C-terminal region comprises, consists of, or consists essentially of, an amino acid sequence at least 90% %, at least 95%, at least 97%, or 100%, identical to SEQ ID NO:38. In certain aspects, the modified CSP lacks an amino acid sequence corresponding to amino acids 1-135, or 18-135, of SEQ ID NO:31.

In certain aspects, a modified CSP of the disclosure comprises, consists of, or consists essentially of, an amino acid sequence at least at least 90% %, at least 95%, at least 97%, or at least 99% identical to SEQ ID NO:32, wherein the modified CSP comprises a first amino acid sequence at least 83%, or 100% identical to SEQ ID NO:37 joined to a second amino acid sequence consisting of, or consisting essentially of, three, four, five, six or seven consecutive NANP sequences, wherein the second amino acid sequence is joined to a third amino acid sequence comprising, consisting of, or consisting essentially of, an amino acid sequence at least 90% %, at least 95%, at least 97%, or 100%, identical to SEQ ID NO:38. In certain aspects, a modified CSP of the disclosure comprises, consists of, or consists essentially of SEQ ID NO:32.

One aspect of the disclosure is an isolated protein comprising a modified 6C domain of the disclosure. In certain aspects, the modified 6C domain may comprise an amino acid sequence having at least about 70%, at least about 72%, at least about 74%, at least about 75%, at least about 80%, at least about 85%, at least about 90%, at least about 92%, at least about 94%, at least about 96%, at least about 98%, or at least about 99%, homology over the entire length of a 6C domain from a Plasmodium Plasmodium 48/45 protein, wherein the amino acid sequence comprises one or more mutations relative to the wt Plasmodium 6C amino acid sequence, that increase the stability of the recombinant protein relative to the stability of a second isolated protein comprising the amino acid sequence of the wt Plasmodium 6C domain, wherein the recombinant protein comprise a 85RF45.1 epitope, and wherein the contact residues of the 85RF45.1 epitope are from the amino acid sequence. In certain aspects, the isolated protein may comprise a full-length P48/45 protein, in which the 6C domain may comprise a modified 6C domain of the disclosure.

One aspect of the disclosure is an isolated protein comprising an amino acid sequence having at least about 80%, at least about 85%, at least about 90%, at least about 92%, at least about 94%, at least about 96%, at least about 98%, or at least about 99%, or 100%, homology, or identity, over the entire length of SEQ ID NO:2, SEQ ID NO:3, SEQ ID NO:4, SEQ ID NO:5, SEQ ID NO:6, SEQ ID NO:7, SEQ ID NO: 8, SEQ ID NO:9, SEQ ID NO:10, SEQ ID NO: 11, SEQ ID NO:12, SEQ ID NO: 13, SEQ ID NO:14, SEQ ID NO:15, SEQ ID NO:16, and SEQ ID NO:17.

In certain aspects, the amino acid sequence may comprise at least 10 amino acid residues, at least 15 amino acid residues, at least 20 amino acid residues, at least 21 amino acid residues, at least 22 amino acid residues, at least 23 amino acid residues, at least 24 amino acid residues, or at least 25 amino acid residues selected from the group consisting of an aspartic acid at a position corresponding to position 31 of SEQ ID NO:1 [D321], a proline at a position corresponding to position 55 of SEQ ID NO:1 [P345], a glycine at a position corresponding to position 56 of SEQ ID NO: 1 [G346], an aspartic acid at a position corresponding to position 57 of SEQ ID NO: 1 [D347], a isoleucine at a position corresponding to position 58 of SEQ ID NO:1 [I348], a isoleucine at a position corresponding to position 59 of SEQ ID NO:1 [1349], a proline at a position corresponding to position 60 of SEQ ID NO:1 [P350], an aspartic acid at a position corresponding to position 61 of SEQ ID NO:1 [D351], a phenylalanine at a position corresponding to position 64 of SEQ ID NO:1 [F354], a glutamine at a position corresponding to position 65 of SEQ ID NO:1 [Q355], a leucine at a position corresponding to position 74 of SEQ ID NO:1 [L364], a glutamic acid at a position corresponding to position 75 of SEQ ID NO:1 [E365], a proline at a position corresponding to position 76 of SEQ ID NO:1 [P366], a serine at a position corresponding to position 77 of SEQ ID NO:1 [P367], an isoleucine at a position corresponding to position 79 of SEQ ID NO:1 [1369], a tyrosine at apposition corresponding to position 81 of SEQ ID NO: 3 [Y371], a glutamic acid at a position corresponding to position 95 of SEQ ID NO:1 [E385], an aspartic acid a position corresponding to position 100 of SEQ ID NO:1 [D390], a lysine at a position corroding to position 102 of SEQ ID NO:1 [K392], a lysine at a position corresponding to position 104 of SEQ ID NO:1 [K394], an isoleucine at a position corresponding to position 121 of SEQ ID NO:1 [1411], a lysine at a position corresponding to position 123 of SEQ ID NO: 1 [K413], a lysine at a position corresponding to position 124 of SEQ ID NO:1 [K414], an aspartic acid at a position corresponding to position 125 of SEQ ID NO:1 [D415], a lysine at a position corresponding to position 126 of SEQ ID NO:1 [K416], and a serine at a position corresponding to position 128 of SEQ ID NO:1 [S418].

In certain aspects, the amino acid sequence may comprise an aspartic acid at a position corresponding to position 31 of SEQ ID NO:1 [D321], a proline at a position corresponding to position 55 of SEQ ID NO:1 [P345], a glycine at a position corresponding to position 56 of SEQ ID NO:1 [G346], an aspartic acid at a position corresponding to position 57 of SEQ ID NO:1 [D347], a isoleucine at a position corresponding to position 58 of SEQ ID NO:1 [1348], a isoleucine at a position corresponding to position 59 of SEQ ID NO:1 [1349], a proline at a position corresponding to position 60 of SEQ ID NO:1 [P350], an aspartic acid at a position corresponding to position 61 of SEQ ID NO:1 [D351], a phenylalanine at a position corresponding to position 64 of SEQ ID NO:1 [F354], a glutamine at a position corresponding to position 65 of SEQ ID NO:1 [Q355], a leucine at a position corresponding to position 74 of SEQ ID NO:1 [L364], a glutamic acid at a position corresponding to position 75 of SEQ ID NO:1 [E365], a proline at a position corresponding to position 76 of SEQ ID NO:1 [P366], a serine at a position corresponding to position 77 of SEQ ID NO:1 [P367], an isoleucine at a position corresponding to position 79 of SEQ ID NO:1 [1369], a tyrosine at apposition corresponding to position 81 of SEQ ID NO:1 [Y371], a lysine at a position corroding to position 102 of [K392], a lysine at a position corresponding to position 104 of SEQ ID NO: 1 [K394], an isoleucine at a position corresponding to position 121 of SEQ ID NO:1 [I411], a lysine at a position corresponding to position 123 of SEQ ID NO:1 [K413], a lysine at a position corresponding to position 124 of SEQ ID NO:1 [K414], an aspartic acid at a position corresponding to position 125 of SEQ ID NO:1 [D415], a lysine at a position corresponding to position 126 of SEQ ID NO:1 [K416], and a serine at a position corresponding to position 128 of SEQ ID NO:1 [S418]. In certain aspects, the amino acid sequence may, optionally, comprise a glutamic acid at a position corresponding to position 95 of SEQ ID NO:1 [E385] and/or an aspartic acid a position corresponding to position 100 of SEQ ID NO:1 [D390].

In certain aspects, the amino acid sequence may comprise at least 10 amino acid residues, at least 15 amino acid residues, at least 20 amino acid residues, at least 21 amino acid residues, at least 22 amino acid residues, at least 23 amino acid residues, at least 24 amino acid residues, or at least 25 amino acid residues selected from the group consisting of D31, P55, G56, D57, 158, 159, P60, D61, F64, Q65, L74, E75, P76, S77, 179, Y81, E95, D100, K102, K104, 1121, K123, K124, D125, K126, and S128 of SEQ ID NO:2 In certain aspects, the amino acid sequence may comprise D31, P55, G56, D57, 158, 159, P60, D61, F64, Q65, L74, E75, P76, S77, I79, Y81, K102, K104, 1121, K123, K124, D125, K126, and S128 of SEQ ID NO:2, and, optionally, E95 and/or D100, of SEQ ID NO:2.

In certain aspects, the amino acid sequence may comprise at least 10 amino acid residues, at least 15 amino acid residues, at least 20 amino acid residues, at least 21 amino acid residues, at least 22 amino acid residues, at least 23 amino acid residues, at least 24 amino acid residues, or at least 25 amino acid residues selected from the group consisting of an aspartic acid at position 31, a proline at position 55, a glycine at position 56, an aspartic acid at position 57, an isoleucine at position 58, an isoleucine at position 59, a proline at position 60, an aspartic acid at position 61, a phenylalanine at position 64, a glutamine at position 65, a leucine at position 74, a glutamic acid at position 75, a proline at position 76, a serine at position 77, an isoleucine at position 79, a tyrosine at position 81, a glutamic acid at position 95, an aspartic acid at position 100, a lysine at position 102, a lysine at position 104, an isoleucine at position 121, a lysine at position 123, a lysine at position 124, an aspartic acid at position 125, a lysine at position 126, and a serine at position 128. In certain aspects, the amino acid sequence may comprise an aspartic acid at position 31, a proline at position 55, a glycine at position 56, an aspartic acid at position 57, an isoleucine at position 58, an isoleucine at position 59, a proline at position 60, an aspartic acid at position 61, a phenylalanine at position 64, a glutamine at position 65, a leucine at position 74, a glutamic acid at position 75, a proline at position 76, a serine at position 77, an isoleucine at position 79, a tyrosine at position 81, a lysine at position 102, a lysine at position 104, an isoleucine at position 121, a lysine at position 123, a lysine at position 124, an aspartic acid at position 125, a lysine at position 126, and a serine at position 128, and, optionally, a glutamic acid at position 95 and/or an aspartic acid at position 100.

### Immunogen 15 Mutations

In certain aspects, the amino acid sequence differs from the SEQ ID NO: 2 at one or more amino acid positions selected from the group consisting amino acid position 3, amino acid position 6, amino acid position 11, amino acid position 14, amino acid position 15, amino acid position 18, amino acid position 19, amino acid, amino acid position 21, amino acid position 22, amino acid position 28, amino acid position 33, amino acid position 35, amino acid position 36, amino acid position 38, amino acid position 43, amino acid position 45, amino acid position 49, amino acid position 68, amino acid position 69, amino acid position 70, amino acid position 71, amino acid position 83, amino acid position 84, amino acid position 86, amino acid position 89, amino acid position 90, amino acid position 97, amino acid position 105, amino acid position 107, amino acid position 109, amino acid position 114, amino acid position 115, amino acid position 134, and amino acid position 138, of SEQ ID NO:2. In certain aspects, the amino acid sequence may comprise one or more amino acids selected from the group consisting of a serine at a position corresponding to position 3 of SEQ ID NO:2, an alanine at a position corresponding to position 6 of SEQ ID NO:2, an alanine at a position corresponding to position 11 of SEQ ID NO:2, a lysine at a position corresponding to position 14 of SEQ ID NO:2, an asparagine at a position corresponding to position 15 of SEQ ID NO:2, a tyrosine at a position corresponding to position 18 of SEQ ID NO:2, a lysine at a position corresponding to position 19 of SEQ ID NO:2, an alanine at a position corresponding to position 21 of SEQ ID NO:2, a lysine at a position corresponding to position 22 of SEQ ID NO:2, a glutamine at a position corresponding to position 28 of SEQ ID NO:2, a lysine at a position corresponding to position 33 of SEQ ID NO: 2, a valine at a position corresponding to position 35 of SEQ ID NO:2, an as arginine at a position corresponding to position 36 of SEQ ID NO:2, a glutamic acid at a position corresponding to position 38 of SEQ ID NO:2, a histidine at a position corresponding to position 43 of SEQ ID NO:2, a glutamic acid at a position corresponding to position 45 of SEQ ID NO:2, an isoleucine at a position corresponding to position 49 of SEQ ID NO:2, histidine at a position corresponding to position 68 of SEQ ID NO:2, an asparagine at a position corresponding to position 69 of SEQ ID NO:2, a serine at a position corresponding to position 70 of SEQ ID NO:2, an alanine at a position corresponding to position 71 of SEQ ID NO:2, a glutamic acid at a position corresponding to position 83 of SEQ ID NO:2, as aspartic acid at a position corresponding to position 84 of SEQ ID NO:2, a leucine at a position corresponding to position 86 of SEQ ID NO:2, an alanine at a position corresponding to position 89 of SEQ ID NO:2, an asparagine at a position corresponding to position 90 of SEQ ID NO:2, an arginine at a position corresponding to position 97 of SEQ ID NO:2, a methionine at a position corresponding to position 105 of SEQ ID NO:2, an alanine at a position corresponding to position 107 of SEQ ID NO:2, an isoleucine at a position corresponding to position 109 of SEQ ID NO:2, a glutamic acid at a position corresponding to position 114 of SEQ ID NO:2, a proline at a position corresponding to position 115 of SEQ ID NO:2, a phenylalanine at a position corresponding to position 134 of SEQ ID NO:2, and a serine at a position corresponding to position 138 of SEQ ID NO: 2. In certain aspects, the amino acid sequence may comprise one or more amino acid residues selected from the group consisting of S3, A6, A11, K14, N15, Y18, K19, A21, K22, Q28, K33, V35, R36, E38, H43, E45, 149, H68, N69, S70, A71, E83, D84, L86, A89, N90, R97, M105, A107, 1109, E114, P115, E134, and S138, of SEQ ID NO:2. In certain aspects, the amino acid sequence may comprise one or more amino acid residues selected from the group consisting of a serine at position 3, an alanine at position 6, an alanine at position 11, a lysine at position 14, an asparagine at position 15, a tyrosine at position 18, a lysine at position 19, an alanine at position 21, a lysine at position 22, a glutamine at position 28, a lysine at position 33, a valine at position 35, an as arginine at position 36, a glutamic acid at position 38, a histidine at position 43, a glutamic acid at position 45, an isoleucine at position 49, histidine at position 68, an asparagine at position 69, a serine at position 70, an alanine at position 71, a glutamic acid at position 83, as aspartic acid at position 84, a leucine at position 86, an alanine at position 89, an asparagine at position 90, an arginine at position 97, a methionine at position 105, an alanine at position 107, an isoleucine at position 109, a glutamic acid at position 114, a proline at position 115, a phenylalanine at position 134, and a serine at position 138. In certain aspects, the amino acid sequence may comprise a serine at position 3, an alanine at position 6, an alanine at position 11, a lysine at position 14, an asparagine at position 15, a tyrosine at position 18, a lysine at position 19, an alanine at position 21, a lysine at position 22, a glutamine at position 28, a lysine at position 33, a valine at position 35, an as arginine at position 36, a glutamic acid at position 38, a histidine at position 43, a glutamic acid at position 45, an isoleucine at position 49, histidine at position 68, an asparagine at position 69, a serine at position 70, an alanine at position 71, a glutamic acid at position 83, as aspartic acid at position 84, a leucine at position 86, an alanine at position 89, an asparagine at position 90, an arginine at position 97, a methionine at position 105, an alanine at position 107, an isoleucine at position 109, a glutamic acid at position 114, a proline at position 115, a phenylalanine at position 134, and a serine at position 138. In certain aspects, the amino acid sequence lacks an n-glycosylation signal. In certain aspects, the amino acid sequence may comprise SEQ ID NO:2.

### Immunogen 15-2 Mutations

In certain aspects, the amino acid sequence differs from SEQ ID NO: 3 at one or more amino acid positions selected from the group consisting of amino acid position 6, amino acid position 11, amino acid position 14, amino acid position 18, amino acid position 19, amino acid position 22, amino acid position 33, amino acid position 36, amino acid position 38, amino acid position 43, amino acid position 49, amino acid position 69, amino acid position 70, amino acid position 71, amino acid position 83, amino acid position 86, amino acid position 89, amino acid position 97, amino acid position 105, amino acid position 107, amino acid position 115, and amino acid position 134. In certain aspects, the amino acid sequence may comprise one or more amino acids selected from the group consisting of an alanine at a position corresponding to position 6 of SEQ ID NO:3, an alanine at a position corresponding to position 11 of SEQ ID NO:3, a lysine at a position corresponding to position 14 of SEQ ID NO: 3, an alanine at a position corresponding to position 15 of SEQ ID NO:3, a tyrosine at a position corresponding to position 18 of SEQ ID NO:3, a lysine at a position corresponding to position 19 of SEQ ID NO:3, a lysine at a position corresponding to position 22 of SEQ ID NO:3, a lysine at a position corresponding to position 33 of SEQ ID NO: 3, an arginine at a position corresponding to position 36 of SEQ ID NO:3, a glutamic acid at a position corresponding to position 38 of SEQ ID NO:3, a histidine at a position corresponding to position 43 of SEQ ID NO:3, an isoleucine at a position corresponding to position 49 of SEQ ID NO:3, an asparagine at a position corresponding to position 69 of SEQ ID NO:3, a serine at a position corresponding to position 70 of SEQ ID NO:3, an alanine at a position corresponding to position 71 of SEQ ID NO:3, a glutamic acid at a position corresponding to position 83 of SEQ ID NO:3, a leucine at a position corresponding to position 86 of SEQ ID NO:3, an alanine at a position corresponding to position 89 of SEQ ID NO:3, an arginine at a position corresponding to position 97 of SEQ ID NO:3, a methionine at a position corresponding to position 105 of SEQ ID NO:3, an alanine at a position corresponding to position 107 of SEQ ID NO:3, a proline at a position corresponding to position 115 of SEQ ID NO:3, and a phenylalanine at a position corresponding to position 134 of SEQ ID NO:3. In certain aspects, the amino acid sequence may comprise one or more amino acid residues selected from the group consisting of A6, A11, K14, A15, Y18, K19, K22, K33, R36, E38, H43, 149, N69, S70, A71, E83, L86, A89, R97, M105, A107, P115, F134, of SEQ ID NO:3. In certain aspects, the amino acid sequence may comprise one or more amino acids selected from the group consisting of an alanine at position 6, an alanine at position 11, a lysine at position 14, an alanine at position 15, a tyrosine at position 18, a lysine at position 19, a lysine at position 22, a lysine at position 33, an arginine at position 36, a glutamic acid at position 38, a histidine at position 43, an isoleucine at position 49, an asparagine at position 69, a serine at position 70, an alanine at position 71, a glutamic acid at a position corresponding to position 83, a leucine at position 86, an alanine at position 89, an arginine at position 97, a methionine at position 105, an alanine at position 107, a proline at position 115, and a phenylalanine at position 134. In certain aspects, the amino acid sequence may comprise an alanine at position 6, an alanine at position 11, a lysine at position 14, an alanine at position 15, a tyrosine at position 18, a lysine at position 19, a lysine at position 22, a lysine at position 33, an arginine at position 36, a glutamic acid at position 38, a histidine at position 43, an isoleucine at position 49, an asparagine at position 69, a serine at position 70, an alanine at position 71, a glutamic acid at a position corresponding to position 83, a leucine at position 86, an alanine at position 89, an arginine at position 97, a methionine at position 105, an alanine at position 107, a proline at position 115, and a phenylalanine at position 134. In certain aspects, the amino acid sequence may comprise SEQ ID NO:3.

### Immunogen 17

In certain aspects, the amino acid sequence differs from SEQ ID NO: 4 at one or more amino acid positions selected from the group consisting of position 3, position 11, position 14, position 15, position 19, position 21, position 22, position 28, position 33, position 35, position 36, position 38, position 40, position 43, position 44, position 45, position 49, position 52,position 68, position 69, position 70, position 84, position 85, position 89, position 97, position 107, position 115, position 134, and position 138, of SEQ ID NO:4. In certain aspects, the amino acid sequence may comprise one or more amino acids selected from the group consisting of a serine at a position corresponding to position 3 of SEQ ID NO:4, an alanine at a position corresponding to position 11 of SEQ ID NO:4, a lysine at a position corresponding to position 14 of SEQ ID NO:4, an asparagine at a position corresponding to position 15 of SEQ ID NO:4, a lysine at a position corresponding to position 19 of SEQ ID NO:4, an alanine at a position corresponding to position 21 of SEQ ID NO:4, a lysine at a position corresponding to position 22 of SEQ ID NO:4, a glutamine at a position corresponding to position 28 of SEQ ID NO:4, a lysine at a position corresponding to position 33 of SEQ ID NO: 4, a valine at a position corresponding to position 35 of SEQ ID NO:4, an arginine at a position corresponding to position 36 of SEQ ID NO:4, a glutamic acid at a position corresponding to position 38 of SEQ ID NO:4, a glutamic acid at a position corresponding to position 40 of SEQ ID NO:4, a histidine at a position corresponding to position 43 of SEQ ID NO:4, a lysine at a position corresponding to position 44 of SEQ ID NO: 4, a tyrosine at a position corresponding to position 45 of SEQ ID NO:4, an isoleucine at a position corresponding to position 49 of SEQ ID NO:4, a methionine at a position corresponding to position 52 of SEQ ID NO:4, a histidine at a position corresponding to position 68 of SEQ ID NO:4, a glutamine at a position corresponding to position 69 of SEQ ID NO:4, a serine at a position corresponding to position 70 of SEQ ID NO: 4, as aspartic acid at a position corresponding to position 84 of SEQ ID NO:4, a valine at a position corresponding to position 85 of SEQ ID NO:4, an aspartic acid at a position corresponding to position 89 of SEQ ID NO:4, an arginine at a position corresponding to position 97 of SEQ ID NO:4, a lysine at a position corresponding to position 107 of SEQ ID NO:4, a proline at a position corresponding to position 115 of SEQ ID NO:4, a lysine at a position corresponding to position 134 of SEQ ID NO:4, and a serine at a position corresponding to position 138 of SEQ ID NO:4. In certain aspects, the amino acid sequence may comprise one or more amin acid residues selected form the group consisting of S3, A11, K14, N15, K19, A21, K22, Q28, K33, V35, R36, E38, E40, H43, K44, Y45, 149, M52, H68, Q69, S70, D84, V85, D89, R97, K107, P115, K134, S138, of SEQ ID NO:4. In certain aspects, the amino acid sequence may comprise one or more amino acids selected from the group consisting of a serine at position 3, an alanine at position 11, a lysine at position 14, an asparagine at position 15, a lysine at position 19, an alanine at position 21, a lysine at position 22, a glutamine at position 28, a lysine at position 33, a valine at position 35, an arginine at position 36, a glutamic acid at position 38, a glutamic acid at position 40, a histidine at position 43, a lysine at position 44, a tyrosine at position 45, an isoleucine at position 49, a methionine at position 52, a histidine at position 68, a glutamine at position 69, a serine at position 70, as aspartic acid at position 84, a valine at position 85, an aspartic acid at position 89, an arginine at position 97, a lysine at position 107, a proline at position 115, a lysine at position 134, and a serine at position 138. In certain aspects, the amino acid sequence may comprise a serine at position 3, an alanine at position 11, a lysine at position 14, an asparagine at position 15, a lysine at position 19, an alanine at position 21, a lysine at position 22, a glutamine at position 28, a lysine at position 33, a valine at position 35, an arginine at position 36, a glutamic acid at position 38, a glutamic acid at position 40, a histidine at position 43, a lysine at position 44, a tyrosine at position 45, an isoleucine at position 49, a methionine at position 52, a histidine at position 68, a glutamine at position 69, a serine at position 70, as aspartic acid at position 84, a valine at position 85, an aspartic acid at position 89, an arginine at position 97, a lysine at position 107, a proline at position 115, a lysine at position 134, and a serine at position 138. In certain aspects, the amino acid sequence may comprise SEQ ID NO:4.

### Immunogen 17-4

In certain aspects, the amino acid sequence differs from SEQ ID NO: 5 at one or more amino acid positions selected from the group consisting of amino acid position 11, amino acid position 15, amino acid position 22, an amino acid position 44, amino acid position 52, amino acid position 69, and amino acid position 107. In certain aspects, the amino acid sequence may comprise one or more amino acid residues selected from the group consisting of an alanine at a position corresponding to position 11 of SEQ ID NO:1, an alanine at a position corresponding to position 15 of SEQ ID NO: 1, a lysine at a position corresponding to position 22 of SEQ ID NO:1, a lysine at a position corresponding to position 44 of SEQ ID NO:1, a methionine at a position corresponding to position 52 of SEQ ID NO:1, a glutamine at a position corresponding to position 69 of SEQ ID NO:1, and a lysine at a position corresponding to position 107 of SEQ ID NO:1. In certain aspects, the amino acid sequence may comprise one or more amino acid residues selected from the group consisting of A11, A15, K22, K44, M52, Q69, K107, of SEQ ID NO:5. In certain aspects, the amino acid sequence may comprise one or more amino acid residues selected from the group consisting of an alanine position 11, an alanine at position 15, a lysine at position 22, a lysine at position 44, a methionine at position 52, a glutamine at position 69, and a lysine at position 107. In certain aspects, the amino acid sequence may comprise an alanine position 11, an alanine at position 15, a lysine at position 22, a lysine at position 44, a methionine at position 52, a glutamine at position 69, and a lysine at position 107.

In certain aspects, the amino acid sequence may comprise at least 10 amino acid residues, at least 15 amino acid residues, at least 20 amino acid residues, at least 21 amino acid residues, at least 22 amino acid residues, at least 23 amino acid residues, at least 24 amino acid residues, or at least 25 amino acid residues selected from the group consisting of an aspartic acid at a position corresponding to position 31 of SEQ ID NO:1 [D321], a proline at a position corresponding to position 55 of SEQ ID NO:1 [P345], a glycine at a position corresponding to position 56 of SEQ ID NO: 1 [G346], an aspartic acid at a position corresponding to position 57 of SEQ ID NO: 1 [D347], a isoleucine at a position corresponding to position 58 of SEQ ID NO:1 [I348], a isoleucine at a position corresponding to position 59 of SEQ ID NO:1 [1349], a proline at a position corresponding to position 60 of SEQ ID NO:1 [P350], an aspartic acid at a position corresponding to position 61 of SEQ ID NO:1 [D351], a phenylalanine at a position corresponding to position 64 of SEQ ID NO:1 [F354], a glutamine at a position corresponding to position 65 of SEQ ID NO:1 [Q355], a leucine at a position corresponding to position 74 of SEQ ID NO:1 [L364], a glutamic acid at a position corresponding to position 75 of SEQ ID NO:1 [E365], a proline at a position corresponding to position 76 of SEQ ID NO:1 [P366], a serine at a position corresponding to position 77 of SEQ ID NO:1 [P367], an isoleucine at a position corresponding to position 79 of SEQ ID NO:1 [1369], a tyrosine at apposition corresponding to position 81 of SEQ ID NO: 3 [Y371], a glutamic acid at a position corresponding to position 95 of SEQ ID NO:1 [E385], an aspartic acid a position corresponding to position 100 of SEQ ID NO:1 [D390], a lysine at a position corroding to position 102 of SEQ ID NO:1 [K392], a lysine at a position corresponding to position 104 of SEQ ID NO:1 [K394], an isoleucine at a position corresponding to position 121 of SEQ ID NO:1 [1411], a lysine at a position corresponding to position 123 of SEQ ID NO: 1 [K413], a lysine at a position corresponding to position 124 of SEQ ID NO:1 [K414], an aspartic acid at a position corresponding to position 125 of SEQ ID NO:1 [D415], a lysine at a position corresponding to position 126 of SEQ ID NO:1 [K416], and a serine at a position corresponding to position 128 of SEQ ID NO:1 [S418].

In certain aspects, the amino acid sequence may comprise an aspartic acid at a position corresponding to position 31 of SEQ ID NO:1 [D321], a proline at a position corresponding to position 55 of SEQ ID NO:1 [P345], a glycine at a position corresponding to position 56 of SEQ ID NO:1 [G346], an aspartic acid at a position corresponding to position 57 of SEQ ID NO:1 [D347], a isoleucine at a position corresponding to position 58 of SEQ ID NO:1 [1348], a isoleucine at a position corresponding to position 59 of SEQ ID NO:1 [1349], a proline at a position corresponding to position 60 of SEQ ID NO:1 [P350], an aspartic acid at a position corresponding to position 61 of SEQ ID NO:1 [D351], a phenylalanine at a position corresponding to position 64 of SEQ ID NO:1 [F354], a glutamine at a position corresponding to position 65 of SEQ ID NO:1 [Q355], a leucine at a position corresponding to position 74 of SEQ ID NO:1 [L364], a glutamic acid at a position corresponding to position 75 of SEQ ID NO:1 [E365], a proline at a position corresponding to position 76 of SEQ ID NO:1 [P366], a serine at a position corresponding to position 77 of SEQ ID NO:1 [P367], an isoleucine at a position corresponding to position 79 of SEQ ID NO:1 [1369], a tyrosine at apposition corresponding to position 81 of SEQ ID NO:1 [Y371], a lysine at a position corroding to position 102 of [K392], a lysine at a position corresponding to position 104 of SEQ ID NO: 1 [K394], an isoleucine at a position corresponding to position 121 of SEQ ID NO:1 [I411], a lysine at a position corresponding to position 123 of SEQ ID NO:1 [K413], a lysine at a position corresponding to position 124 of SEQ ID NO:1 [K414], an aspartic acid at a position corresponding to position 125 of SEQ ID NO:1 [D415], a lysine at a position corresponding to position 126 of SEQ ID NO:1 [K416], and a serine at a position corresponding to position 128 of SEQ ID NO:1 [S418]. In certain aspects, a modified 6C domain of the disclosure may also, optionally, comprise a glutamic acid at a position corresponding to position 95 of SEQ ID NO:1 [E385] and/or an aspartic acid a position corresponding to position 100 of SEQ ID NO:1 [D390].

### Immunogen 27

In certain aspects, the amino acid sequence differs from SEQ ID NO: 1 at one or more amino acid positions selected from the group consisting of an amino acid position corresponding to position 3 of SEQ ID NO:1, an amino acid position corresponding to position 4 of SEQ ID NO:1, an amino acid position corresponding to position 6 of SEQ ID NO:1, an amino acid position corresponding to position 11 of SEQ ID NO: 1, an amino acid position corresponding to position 14 of SEQ ID NO:1, an amino acid position corresponding to position 15 of SEQ ID NO:1, an amino acid position corresponding to position 18 of SEQ ID NO:1, an amino acid position corresponding to position 19 of SEQ ID NO:1, an amino acid position corresponding to position 21 of SEQ ID NO:1, an amino acid position corresponding to position 22 of SEQ ID NO:1, an amino acid position corresponding to position 28 of SEQ ID NO:1, an amino acid position corresponding to position 36 of SEQ ID NO:1, an amino acid position corresponding to position 43 of SEQ ID NO:1, an amino acid position corresponding to position 49 of SEQ ID NO:1, an amino acid position corresponding to position 70 of SEQ ID NO:1, an amino acid position corresponding to position 85 of SEQ ID NO:1, an amino acid position corresponding to position 89 of SEQ ID NO:1, an amino acid position corresponding to position 90 of SEQ ID NO:1, an amino acid position corresponding to position 97 of SEQ ID NO:1, an amino acid position corresponding to position 105 of SEQ ID NO:1, an amino acid position corresponding to position 107 of SEQ ID NO:1, an amino acid position corresponding to position 115 of SEQ ID NO:1, an amino acid position corresponding to position 134 of SEQ ID NO:1, and an amino acid position corresponding to position 138 of SEQ ID NO:1.

In certain aspects, the amino acid sequence may comprise one or more amino acid residues selected from the group consisting of a serine at a position corresponding to position 3 of SEQ ID NO:1, a threonine at a position corresponding to position 4 of SEQ ID NO:1, an arginine at a position corresponding to position 6 of SEQ ID NO: 1, an alanine at a position corresponding to position 11 of SEQ ID NO:1, a lysine at a position corresponding to position 14 of SEQ ID NO:1, an asparagine at a position corresponding to position 15 of SEQ ID NO:1, a tryptophan at a position corresponding to position 18 of SEQ ID NO:1, a glutamine at a position corresponding to position 19 of SEQ ID NO:1, an aspartic acid at a position corresponding to position 21 of SEQ ID NO: 1, an asparagine at a position corresponding to position 22 of SEQ ID NO:1, a glutamine at a position corresponding to position 28 of SEQ ID NO:1, an arginine at a position corresponding to position 36 of SEQ ID NO:1, a tyrosine at a position corresponding to position 43 of SEQ ID NO:1, an isoleucine at a position corresponding to position 49 of SEQ ID NO:1, a serine at a position corresponding to position 70 of SEQ ID NO:1, as aspartic acid at a position corresponding to position 84 of SEQ ID NO: 1, a valine at a position corresponding to position 85 of SEQ ID NO:1, an aspartic acid at a position corresponding to position 89 of SEQ ID NO:1, an asparagine at a position corresponding to position 90 of SEQ ID NO:1,an arginine at a position corresponding to position 97 of SEQ ID NO:1, a methionine at a position corresponding to position 105 of SEQ ID NO:1, a lysine at a position corresponding to position 107 of SEQ ID NO:1, a proline at a position corresponding to position 115 of SEQ ID NO:1, a lysine at a position corresponding to position 134 of SEQ ID NO:1, and a serine at a position corresponding to position 138 of SEQ ID NO:1.

In certain aspects, the amino acid sequence may comprise at least 10 amino acid residues, at least 15 amino acid residues, at least 20 amino acid residues, at least 21 amino acid residues, at least 22 amino acid residues, or at least 23 residues selected from the group consisting of S3, T4, R6, A11, K14, N15, W18, Q19, D21, N22, Q28, R36, Y43, 149, S70, V85, D89, N90, R97, M105, K107, P115, E134, and S138, of SEQ ID NO:6. In certain aspects, the amino acid sequence may comprise S3, T4, R6, A11, K14, N15, W18, Q19, D21, N22, Q28, R36, Y43, 149, S70, V85, D89, N90, R97, M105, K107, P115, E134, and S138, of SEQ ID NO:6. In certain aspects, the amino acid sequence may comprise one or more amino acids selected from the group consisting of a serine at position 3, a threonine at position 4, an arginine at position 6, an alanine at position 11, a lysine at position 14, an asparagine at position 15, a tryptophan at position 18, a glutamine at position 19, an aspartic acid at position 21, an asparagine at position 22, a glutamine at position 28, a tyrosine at position 43, an isoleucine at position 49, a serine at position 70, a valine at position 85, an aspartic acid at position 89, an asparagine at position 90, an arginine at position 97, aa methionine at position 105, lysine at position 107, a proline at position 115, a glutamic acid at position 134, and a serine at position 138. In certain aspects, the amino acid sequence may comprise a serine at position 3, a threonine at position 4, an arginine at position 6, an alanine at position 11, a lysine at position 14, an asparagine at position 15, a tryptophan at position 18, a glutamine at position 19, an aspartic acid at position 21, an asparagine at position 22, a glutamine at position 28, a tyrosine at position 43, an isoleucine at position 49, a serine at position 70, a valine at position 85, an aspartic acid at position 89, an asparagine at position 90, an arginine at position 97, aa methionine at position 105, lysine at position 107, a proline at position 115, a glutamic acid at position 134, and a serine at position 138. In certain aspects, the amino acid sequence may comprise SEQ ID NO:6.

### Immunogen 15-1.5

In certain aspects, the amino acid sequence may differ from SEQ ID NO: 1 at one or more amino acid positions selected from the group consisting of an amino acid position corresponding to position 6 of SEQ ID NO:1, an amino acid position corresponding to position 12 of SEQ ID NO:1, an amino acid position corresponding to position 14 of SEQ ID NO:1, an amino acid position corresponding to position 15 of SEQ ID NO:1, an amino acid position corresponding to position 18 of SEQ ID NO:1, an amino acid position corresponding to position 19 of SEQ ID NO:1, an amino acid position corresponding to position 21 of SEQ ID NO:1, an amino acid position corresponding to position 22 of SEQ ID NO:1, an amino acid position corresponding to position 28 of SEQ ID NO:1, an amino acid position corresponding to position 33 of SEQ ID NO:1, an amino acid position corresponding to position 36 of SEQ ID NO:1, an amino acid position corresponding to position 38 of SEQ ID NO:1, an amino acid position corresponding to position 43 of SEQ ID NO:1, an amino acid position corresponding to position 49 of SEQ ID NO:1, an amino acid position corresponding to position 69 of SEQ ID NO:1, an amino acid position corresponding to position 70 of SEQ ID NO:1, an amino acid position corresponding to position 71 of SEQ ID NO:1, an amino acid position corresponding to position 83 of SEQ ID NO:1, an amino acid position corresponding to position 86 of SEQ ID NO:1, an amino acid position corresponding to position 89 of SEQ ID NO:1, an amino acid position corresponding to position 97 of SEQ ID NO:1, an amino acid position corresponding to position 105 of SEQ ID NO:1, an amino acid position corresponding to position 107 of SEQ ID NO:1, an amino acid position corresponding to position 115 of SEQ ID NO:1, and an amino acid position corresponding to position 134 of SEQ ID NO:1.

In certain aspects, the amino acid sequence may comprise one or more amino acids selected from the group consisting of an alanine at a position corresponding to position 6 of SEQ ID NO:1, an alanine at a position corresponding to position 12 of SEQ ID NO:1, a lysine at a position corresponding to position 14 of SEQ ID NO: 1, an alanine at a position corresponding to position 15 of SEQ ID NO:1, a tyrosine at a position corresponding to position 18 of SEQ ID NO:1, a lysine at a position corresponding to position 19 of SEQ ID NO:1, an alanine at a position corresponding to position 21 of SEQ ID NO:1, a lysine at a position corresponding to position 22 of SEQ ID NO: 1, a glutamine at a position corresponding to position 28 of SEQ ID NO:1, a lysine at a position corresponding to position 33 of SEQ ID NO:1, an arginine at a position corresponding to position 36 of SEQ ID NO:1, a glutamic acid at a position corresponding to position 38 of SEQ ID NO:1, a histidine at a position corresponding to position 43 of SEQ ID NO:1, an isoleucine at a position corresponding to position 49 of SEQ ID NO:1, an asparagine at a position corresponding to position 69 of SEQ ID NO:1, a serine at a position corresponding to position 70 of SEQ ID NO:1, an alanine at a position corresponding to position 71 of SEQ ID NO:1, glutamic acid at a position corresponding to position 83 of SEQ ID NO:1, a leucine at a position corresponding to position 86 of SEQ ID NO:1, an alanine at a position corresponding to position 89 of SEQ ID NO:1, an arginine at a position corresponding to position 97 of SEQ ID NO:1, a methionine at a position corresponding to position 105 of SEQ ID NO:1, an alanine at a position corresponding to position 107 of SEQ ID NO:1, a proline at a position corresponding to position 115 of SEQ ID NO:1, and a phenylalanine at a position corresponding to position 134 of SEQ ID NO:1.

In certain aspects, the amino acid sequence may comprise at least 10 amino acid residues, at least 15 amino acid residues, at least 20 amino acid residues, at least 21 amino acid residues, at least 22 amino acid residues, at least 23 amino acid residues, or at least 24 amino acid residues selected from the group consisting of A6, A12, K14, A15, Y18, K19, A21, K22, Q28, K33, R36, E38, H43, 149, N69, S70, A71, E83, L86, A89, R97, M105, A107, P115, and F134, of SEQ ID NO:7. In certain aspects, the amino acid sequence may comprise A6, A12, K14, A15, Y18, K19, A21, K22, Q28, K33, R36, E38, H43, 149, N69, S70, A71, E83, L86, A89, R97, M105, A107, P115, and F134, of SEQ ID NO:7. In certain aspects, the amino acid sequence may comprise one or more amino acids selected from the group consisting of an alanine at position 6, an alanine at position 12, a lysine at position 14, an alanine at position 15, a tyrosine at position 18, a lysine at position 19, an alanine at position 21, a lysine at position 22, a glutamine at position 28, a lysine at position 33, an arginine at position 36, a glutamic acid at position 38, a histidine at position 43, an isoleucine at position 49, an asparagine at position 69, a serine at position 70, an alanine at position 71, a glutamic acid at position 83, a leucine at position 86, an alanine at position 89, an arginine at position 97, a methionine at position 105, lysine at position 107, a proline at position 115, and a phenylalanine at position 134. In certain aspects, the amino acid sequence may comprise an alanine at position 6, an alanine at position 12, a lysine at position 14, an alanine at position 15, a tyrosine at position 18, a lysine at position 19, an alanine at position 21, a lysine at position 22, a glutamine at position 28, a lysine at position 33, an arginine at position 36, a glutamic acid at position 38, a histidine at position 43, an isoleucine at position 49, an asparagine at position 69, a serine at position 70, an alanine at position 71, a glutamic acid at position 83, a leucine at position 86, an alanine at position 89, an arginine at position 97, a methionine at position 105, lysine at position 107, a proline at position 115, and a phenylalanine at position 134. In certain aspects, the amino acid sequence may comprise SEQ ID NO:7.

### Immunogen 15-3

In certain aspects, amino acid sequence may differ from SEQ ID NO: 1 at one or more amino acid positions selected from the group consisting of an amino acid position corresponding to position 6 of SEQ ID NO:1, an amino acid position corresponding to position 11 of SEQ ID NO:1, an amino acid position corresponding to position 15 of SEQ ID NO:1, an amino acid position corresponding to position 38 of SEQ ID NO:1, an amino acid position corresponding to position 69 of SEQ ID NO:1, an amino acid position corresponding to position 70 of SEQ ID NO:1, an amino acid position corresponding to position 71 of SEQ ID NO:1, an amino acid position corresponding to position 83 of SEQ ID NO:1,an amino acid position corresponding to position 89 of SEQ ID NO:1, an amino acid position corresponding to position 97 of SEQ ID NO:1, an amino acid position corresponding to position 105 of SEQ ID NO:1, an amino acid position corresponding to position 107 of SEQ ID NO:1, an amino acid position corresponding to position 115 of SEQ ID NO:1, and an amino acid position corresponding to position 134 of SEQ ID NO:1.

In certain aspects, the amino acid sequence may comprise one or more amino acids selected from the group consisting of an alanine at a position corresponding to position 6 of SEQ ID NO:1, an alanine at a position corresponding to position 11 of SEQ ID NO:1, an alanine at a position corresponding to position 15 of SEQ ID NO:1, a lysine at a position corresponding to position 22 of SEQ ID NO:1, a glutamic acid at a position corresponding to position 38 of SEQ ID NO:1, an asparagine at a position corresponding to position 69 of SEQ ID NO:1, a serine at a position corresponding to position 70 of SEQ ID NO:1, an alanine at a position corresponding to position 71 of SEQ ID NO:1, glutamic acid at a position corresponding to position 83 of SEQ ID NO:1, an alanine at a position corresponding to position 89 of SEQ ID NO:1, an arginine at a position corresponding to position 97 of SEQ ID NO:1, a methionine at a position corresponding to position 105 of SEQ ID NO:1, an alanine at a position corresponding to position 107 of SEQ ID NO:1, a proline at a position corresponding to position 115 of SEQ ID NO:1, and a phenylalanine at a position corresponding to position 134 of SEQ ID NO:1.

In certain aspects, the amino acid sequence may comprise at least 10 amino acid residues, at least 10 amino acid residues, at least 12 amino acid residues, at least 13 amino acid residues, or at least 15 amino acid residues selected from the group consisting of A6, A11, A15, K22, E38, N69, S70, A71, E83, A89, R97, M105, A107, P115, and F134, of SEQ ID NO:8. In certain aspects, the modified 6C domain may comprise A6, A11, A15K22, E38, N69, S70, A71, E83, A89, R97, M105, A107, P115, and F134, of SEQ ID NO:8. In certain aspects, the amino acid sequence may comprise one or more amino acids selected from the group consisting of an alanine at position 6, an alanine at position 11, an alanine at position 15, a lysine at position 22, a glutamic acid at position 38, an asparagine at position 69, a serine at position 70, an alanine at position 71, a glutamic acid at position 83, an alanine at position 89, an arginine at position 97, a methionine at position 105, an alanine at position 107, a proline at position 115, and a phenylalanine at position 134. In certain aspects, the amino acid sequence may comprise an alanine at position 6, an alanine at position 11, an alanine at position 15, a lysine at position 22, a glutamic acid at position 38, an asparagine at position 69, a serine at position 70, an alanine at position 71, a glutamic acid at position 83, an alanine at position 89, an arginine at position 97, a methionine at position 105, an alanine at position 107, a proline at position 115, and a phenylalanine at position 134. In certain aspects, the amino acid sequence may comprise SEQ ID NO:8.

### Immunogen 15-4

In certain aspects, the amino acid sequence may differ from SEQ ID NO: 1 at one or more amino acid positions selected from the group consisting of an amino acid position corresponding to position 11 of SEQ ID NO:1, an amino acid position corresponding to position 15 of SEQ ID NO:1, an amino acid position corresponding to position 38 of SEQ ID NO:1, an amino acid position corresponding to position 69 of SEQ ID NO:1, an amino acid position corresponding to position 70 of SEQ ID NO:1, an amino acid position corresponding to position 71 of SEQ ID NO:1, an amino acid position corresponding to position 83 of SEQ ID NO:1, an amino acid position corresponding to position 86 of SEQ ID NO:1, an amino acid position corresponding to position 105 of SEQ ID NO:1, an amino acid position corresponding to position 107 of SEQ ID NO:1, and an amino acid position corresponding to position 115 of SEQ ID NO:1.

In certain aspects, the amino acid sequence may comprise one or more amino acids selected from the group consisting of an alanine at a position corresponding to position 11 of SEQ ID NO:1, an alanine at a position corresponding to position 15 of SEQ ID NO:1, a glutamic acid at a position corresponding to position 38 of SEQ ID NO:1, an asparagine at a position corresponding to position 69 of SEQ ID NO: 1, a serine at a position corresponding to position 70 of SEQ ID NO:1, an alanine at a position corresponding to position 71 of SEQ ID NO:1, glutamic acid at a position corresponding to position 83 of SEQ ID NO:1, a leucine at a position corresponding to position 86 of SEQ ID NO:1, an arginine at a position corresponding to position 97 of SEQ ID NO:1, a methionine at a position corresponding to position 105 of SEQ ID NO: 1, an alanine at a position corresponding to position 107 of SEQ ID NO:1, and a proline at a position corresponding to position 115 of SEQ ID NO:1.

In certain aspects, the amino acid sequence may comprise at least 10 amino acid residues, at least 8 amino acid residues, at least 9 amino acid residues, or at least 10 amino acid residues selected from the group consisting of A11, A15, E38, N69, S70, A71, E83, L86, M105, A107, and P115, of SEQ ID NO:9. In certain aspects, the modified 6C domain may comprise A11, A15, E38, N69, S70, A71, E83, L86, M105, A107, and P115, of SEQ ID NO:9. In certain aspects, the amino acid sequence may comprise one or more amino acids selected from the group consisting of an alanine at position 11, an alanine at position 15, a glutamic acid at position 38, an asparagine at position 69, a serine at position 70, an alanine at position 71, a glutamic acid at position 83, a leucine at position 86, a methionine at position 105, an alanine at position 107, and a proline at position 115. In certain aspects, the amino acid sequence may comprise an alanine at position 11, an alanine at position 15, a glutamic acid at position 38, an asparagine at position 69, a serine at position 70, an alanine at position 71, a glutamic acid at position 83, a leucine at position 86, a methionine at position 105, an alanine at position 107, and a proline at position 115. In certain aspects, the amino acid sequence may comprise SEQ ID NO:9.

### Immunogen 15-5

In certain aspects, amino acid sequence may differ from SEQ ID NO: 1 at one or more amino acid positions selected from the group consisting of an amino acid position corresponding to position 11 of SEQ ID NO:1, an amino acid position corresponding to position 15 of SEQ ID NO:1, an amino acid position corresponding to position 38 of SEQ ID NO:1, an amino acid position corresponding to position 69 of SEQ ID NO:1, an amino acid position corresponding to position 71 of SEQ ID NO:1, an amino acid position corresponding to position 86 of SEQ ID NO:1, an amino acid position corresponding to position 105 of SEQ ID NO:1, and an amino acid position corresponding to position 107 of SEQ ID NO:1.

In certain aspects, the amino acid sequence may comprise one or more amino acids selected from the group consisting of an alanine at a position corresponding to position 11 of SEQ ID NO:1, an alanine at a position corresponding to position 15 of SEQ ID NO:1, a glutamic acid at a position corresponding to position 38 of SEQ ID NO:1, an asparagine at a position corresponding to position 69 of SEQ ID NO: 1, an alanine at a position corresponding to position 71 of SEQ ID NO:1, a leucine at a position corresponding to position 86 of SEQ ID NO:1, an arginine at a position corresponding to position 97 of SEQ ID NO:1, a methionine at a position corresponding to position 105 of SEQ ID NO:1, and an alanine at a position corresponding to position 107 of SEQ ID NO:1.

In certain aspects, the amino acid sequence may comprise at least 10 amino acid residues, at least 4 amino acid residues, at least 5 amino acid residues, at least 6 amino acid residues, or at least 7 amino acid residues selected from the group consisting of A11, A15, E38, N69, A71, L86, M105, and A107, of SEQ ID NO: 10. In certain aspects, the modified 6C domain may comprise A11, A15, E38, N69, A71, L86, M105, A107, of SEQ ID NO:10. In certain aspects, the amino acid sequence may comprise one or more amino acids selected from the group consisting of an alanine at position 11, an alanine at position 15, a glutamic acid at position 38, an asparagine at position 69, an alanine at position 71, a leucine at position 86, a methionine at position 105, and an alanine at position 107. In certain aspects, the amino acid sequence may comprise an alanine at position 11, an alanine at position 15, a glutamic acid at position 38, an asparagine at position 69, an alanine at position 71, a leucine at position 86, a methionine at position 105, and an alanine at position 107. In certain aspects, the amino acid sequence may comprise SEQ ID NO:10.

### Immunogen 15-6

In certain aspects, amino acid sequence may differ from SEQ ID NO: 1 at one or more amino acid positions selected from the group consisting of an amino acid position corresponding to position 11 of SEQ ID NO:1, an amino acid position corresponding to position 15 of SEQ ID NO:1, an amino acid position corresponding to position 69 of SEQ ID NO:1, an amino acid position corresponding to position 71 of SEQ ID NO:1, and an amino acid position corresponding to position 105 of SEQ ID NO:1.

In certain aspects, the amino acid sequence may comprise one or more amino acids selected from the group consisting of an alanine at a position corresponding to position 11 of SEQ ID NO:1, an alanine at a position corresponding to position 15 of SEQ ID NO:1, an asparagine at a position corresponding to position 69 of SEQ ID NO:1, an alanine at a position corresponding to position 71 of SEQ ID NO:1, and a methionine at a position corresponding to position 105 of SEQ ID NO:1.

In certain aspects, the amino acid sequence may comprise at least 10 amino acid residues, at least 3 amino acid residues, or at least 4 amino acid residues selected from the group consisting of A11, A15, N69, A71, and M105, of SEQ ID NO: 11. In certain aspects, the modified 6C domain may comprise A11, A15, N69, A71, and M105, of SEQ ID NO:11. In certain aspects, the amino acid sequence may comprise one or more amino acids selected from the group consisting of an alanine at position 11, an alanine at position 15, an asparagine at position 69, an alanine at position 71, and a methionine at position 105. In certain aspects, the amino acid sequence may comprise an alanine at position 11, an alanine at position 15, an asparagine at position 69, an alanine at position 71, and a methionine at position 105. In certain aspects, the amino acid sequence may comprise SEQ ID NO:11.

### Immunogen 15-7

In certain aspects, amino acid sequence may differ from SEQ ID NO: 1 at one or more amino acid positions selected from the group consisting of an amino acid position corresponding to position 11 of SEQ ID NO:1, an amino acid position corresponding to position 15 of SEQ ID NO:1, an amino acid position corresponding to position 69 of SEQ ID NO:1, and an amino acid position corresponding to position 71 of SEQ ID NO:1.

In certain aspects, the amino acid sequence may comprise one or more amino acids selected from the group consisting of an alanine at a position corresponding to position 11 of SEQ ID NO:1, an alanine at a position corresponding to position 15 of SEQ ID NO:1, an asparagine at a position corresponding to position 69 of SEQ ID NO:1, and an alanine at a position corresponding to position 71 of SEQ ID NO: 1.

In certain aspects, the amino acid sequence may comprise at least 10 amino acid residues, at least 2 amino acid residues, or at least 3 amino acid residues selected from the group consisting of A11, A15, N69, and A71, of SEQ ID NO: 11. In certain aspects, the amino acid sequence may comprise A11, A15, N69, and A71, of SEQ ID NO:12. In certain aspects, the amino acid sequence may comprise one or more amino acids selected from the group consisting of an alanine at position 11, an alanine at position 15, an asparagine at position 69, and an alanine at position 71. In certain aspects, the amino acid sequence may comprise an alanine at position 11, an alanine at position 15, an asparagine at position 69, and an alanine at position 71. In certain aspects, the amino acid sequence may comprise SEQ ID NO:12.

### Immunogen 17-1.5

In certain aspects, amino acid sequence may differ from SEQ ID NO: 1 at one or more amino acid positions selected from the group consisting of an amino acid position corresponding to position 11 of SEQ ID NO:1, an amino acid position corresponding to position 14 of SEQ ID NO:1, an amino acid position corresponding to position 15 of SEQ ID NO:1, an amino acid position corresponding to position 16 of SEQ ID NO:1, an amino acid position corresponding to position 19 of SEQ ID NO:1, an amino acid position corresponding to position 21 of SEQ ID NO:1, an amino acid position corresponding to position 22 of SEQ ID NO:1, an amino acid position corresponding to position 28 of SEQ ID NO:1, an amino acid position corresponding to position 33 of SEQ ID NO:1, an amino acid position corresponding to position 36 of SEQ ID NO:1, an amino acid position corresponding to position 38 of SEQ ID NO:1, an amino acid position corresponding to position 40 of SEQ ID NO:1, an amino acid position corresponding to position 43 of SEQ ID NO:1, an amino acid position corresponding to position 44 of SEQ ID NO: 1, an amino acid position corresponding to position 49 of SEQ ID NO:1, an amino acid position corresponding to position 52 of SEQ ID NO:1, an amino acid position corresponding to position 69 of SEQ ID NO:1, an amino acid position corresponding to position 70 of SEQ ID NO:1, an amino acid position corresponding to position 85 of SEQ ID NO:1, an amino acid position corresponding to position 89 of SEQ ID NO:1, an amino acid position corresponding to position 97 of SEQ ID NO:1, an amino acid position corresponding to position 107 of SEQ ID NO:1, and an amino acid position corresponding to position 115 of SEQ ID NO:1.

In certain aspects, the amino acid sequence may comprise one or more amino acids selected from the group consisting of an alanine at a position corresponding to position 11 of SEQ ID NO:1, a lysine at a position corresponding to position 14 of SEQ ID NO:1, an alanine at a position corresponding to position 15 of SEQ ID NO:1, a serine at a position corresponding to position 16 of SEQ ID NO:1, a lysine at a position corresponding to position 19 of SEQ ID NO:1, an alanine at a position corresponding to position 21 of SEQ ID NO:1, a lysine at a position corresponding to position 22 of SEQ ID NO:1, a glutamine at a position corresponding to position 28 of SEQ ID NO:1, a lysine at a position corresponding to position 33 of SEQ ID NO: 1, an arginine at a position corresponding to position 36 of SEQ ID NO:1, a glutamic acid at a position corresponding to position 38 of SEQ ID NO:1, a glutamic acid at a position corresponding to position 40 of SEQ ID NO:1, a histidine at a position corresponding to position 43 of SEQ ID NO:1, a lysine at a position corresponding to position 44 of SEQ ID NO:1, an isoleucine at a position corresponding to position 49 of SEQ ID NO:1, a methionine at a position corresponding to position 52 of SEQ ID NO:1, a glutamine at a position corresponding to position 69 of SEQ ID NO:1, a serine at a position corresponding to position 70 of SEQ ID NO:1, a valine at a position corresponding to position 85 of SEQ ID NO:1, an aspartic acid at a position corresponding to position 89 of SEQ ID NO:1, an arginine at a position corresponding to position 97 of SEQ ID NO:1, a lysine at a position corresponding to position 107 of SEQ ID NO: 1, and a proline at a position corresponding to position 115 of SEQ ID NO:1.

In certain aspects, the amino acid sequence may comprise at least 10 amino acid residues, at least 18 amino acid residues, at least 20 amino acid residues, at least 21 amino acid residues, or at least 22 amino acid residues, selected from the group consisting of A11, K14, A15, S16, K19, A21, K22, Q28, K33, R36, E38, E40, H43, K44, 149, M52, Q69, S70, V85, D89, R97, K107, and P115, of SEQ ID NO: 13. In certain aspects, the amino acid sequence may comprise A11, K14, A15, S16, K19, A21, K22, Q28, K33, R36, E38, E40, H43, K44, 149, M52, Q69, $70, V85, D89, R97, K107, and P115, of SEQ ID NO:13. In certain aspects, the amino acid sequence may comprise one or more amino acids selected from the group consisting of an alanine at position 11, a lysine at position 14, an alanine at position 15, serine at position 16, a lysine at position 19, an alanine at position 21, a lysine at position 22, a glutamine at position 28, a lysine at position 33, an arginine at position 36, a glutamic acid at position 38, a glutamic acid at position 40, a histidine at position 43, a lysine at position 44, an isoleucine at position 49, a methionine at position 52, a glutamine at position 69, a serine at position 70, a valine at position 85, an aspartic acid at position 89, an arginine at position 97, a lysine at position 107, and a proline at position 115. In certain aspects, the amino acid sequence may comprise an alanine at position 11, a lysine at position 14, an alanine at position 15, serine at position 16, a lysine at position 19, an alanine at position 21, a lysine at position 22, a glutamine at position 28, a lysine at position 33, an arginine at position 36, a glutamic acid at position 38, a glutamic acid at position 40, a histidine at position 43, a lysine at position 44, an isoleucine at position 49, a methionine at position 52, a glutamine at position 69, a serine at position 70, a valine at position 85, an aspartic acid at position 89, an arginine at position 97, a lysine at position 107, and a proline at position 115. In certain aspects, the amino acid sequence may comprise SEQ ID NO:13.

### Immunogen 17-2

In certain aspects, amino acid sequence may differ from SEQ ID NO: 1 at one or more amino acid positions selected from the group consisting of an amino acid position corresponding to position 11 of SEQ ID NO:1, an amino acid position corresponding to position 14 of SEQ ID NO:1, an amino acid position corresponding to position 15 of SEQ ID NO:1, an amino acid position corresponding to position 19 of SEQ ID NO:1, an amino acid position corresponding to position 22 of SEQ ID NO:1, an amino acid position corresponding to position 33 of SEQ ID NO:1, an amino acid position corresponding to position 36 of SEQ ID NO:1, an amino acid position corresponding to position 38 of SEQ ID NO:1, an amino acid position corresponding to position 40 of SEQ ID NO:1, an amino acid position corresponding to position 43 of SEQ ID NO:1, an amino acid position corresponding to position 44 of SEQ ID NO:1, an amino acid position corresponding to position 69 of SEQ ID NO:1, an amino acid position corresponding to position 70 of SEQ ID NO:1, an amino acid position corresponding to position 85 of SEQ ID NO:1, an amino acid position corresponding to position 89 of SEQ ID NO:1, an amino acid position corresponding to position 97 of SEQ ID NO:1, an amino acid position corresponding to position 107 of SEQ ID NO:1, and an amino acid position corresponding to position 115 of SEQ ID NO: 1.

In certain aspects, the amino acid sequence may comprise one or more amino acids selected from the group consisting of an alanine at a position corresponding to position 11 of SEQ ID NO:1, a lysine at a position corresponding to position 14 of SEQ ID NO:1, an alanine at a position corresponding to position 15 of SEQ ID NO:1, a lysine at a position corresponding to position 19 of SEQ ID NO:1, a lysine at a position corresponding to position 22 of SEQ ID NO:1, a lysine at a position corresponding to position 33 of SEQ ID NO:1, an arginine at a position corresponding to position 36 of SEQ ID NO:1, a glutamic acid at a position corresponding to position 38 of SEQ ID NO:1, a glutamic acid at a position corresponding to position 40 of SEQ ID NO: 1, a histidine at a position corresponding to position 43 of SEQ ID NO:1, a lysine at a position corresponding to position 44 of SEQ ID NO:1, a glutamine at a position corresponding to position 69 of SEQ ID NO:1, a serine at a position corresponding to position 70 of SEQ ID NO:1, a valine at a position corresponding to position 85 of SEQ ID NO: 1, an aspartic acid at a position corresponding to position 89 of SEQ ID NO:1, an arginine at a position corresponding to position 97 of SEQ ID NO:1, a lysine at a position corresponding to position 107 of SEQ ID NO:1, and a proline at a position corresponding to position 115 of SEQ ID NO:1.

In certain aspects, the amino acid sequence may comprise at least 10 amino acid residues, at least 15 amino acid residues, at least 16 amino acid residues, or at least 17 amino acid residues selected from the group consisting of A11, K14, A15, K19, K22, K33, R36, E38, E40, H43, K44, Q69, S70, V85, D89, R97, K107, and P115, of SEQ ID NO:14. In certain aspects, the modified 6C domain may comprise A11, K14, A15, K19, K22, K33, R36, E38, E40, H43, K44, Q69, S70, V85, D89, R97, K107, and P115, of SEQ ID NO:14. In certain aspects, the amino acid sequence may comprise one or more amino acids selected from the group consisting of an alanine at position 11, a lysine at position 14, an alanine at position 15, a lysine at position 19, a lysine at position 22, a lysine at position 33, an arginine at position 36, a glutamic acid at position 38, a glutamic acid at position 40, a histidine at position 43, a lysine at position 44, a glutamine at position 69, a serine at position 70, a valine at position 85, an aspartic acid at position 89, an arginine at position 97, a lysine at position 107, and a proline at position 115. In certain aspects, the amino acid sequence may comprise an alanine at position 11, a lysine at position 14, an alanine at position 15, a lysine at position 19, a lysine at position 22, a lysine at position 33, an arginine at position 36, a glutamic acid at position 38, a glutamic acid at position 40, a histidine at position 43, a lysine at position 44, a glutamine at position 69, a serine at position 70, a valine at position 85, an aspartic acid at position 89, an arginine at position 97, a lysine at position 107, and a proline at position 115. In certain aspects, the amino acid sequence may comprise SEQ ID NO:14.

### Immunogen 17-3

In certain aspects, amino acid sequence may differ from SEQ ID NO: 1 at one or more amino acid positions selected from the group consisting of an amino acid position corresponding to position 11 of SEQ ID NO:1, an amino acid position corresponding to position 15 of SEQ ID NO:1, an amino acid position corresponding to position 22 of SEQ ID NO:1, an amino acid position corresponding to position 38 of SEQ ID NO:1, an amino acid position corresponding to position 43 of SEQ ID NO:1, an amino acid position corresponding to position 44 of SEQ ID NO:1, an amino acid position corresponding to position 49 of SEQ ID NO:1, an amino acid position corresponding to position 52 of SEQ ID NO:1, an amino acid position corresponding to position 69 of SEQ ID NO:1, an amino acid position corresponding to position 70 of SEQ ID NO:1, an amino acid position corresponding to position 85 of SEQ ID NO:1, an amino acid position corresponding to position 89 of SEQ ID NO:1, an amino acid position corresponding to position 97 of SEQ ID NO:1, and an amino acid position corresponding to position 107 of SEQ ID NO:1.

In certain aspects, the amino acid sequence may comprise may comprise one or more amino acids selected from the group consisting of an alanine at a position corresponding to position 11 of SEQ ID NO:1, an alanine at a position corresponding to position 15 of SEQ ID NO:1, a lysine at a position corresponding to position 22 of SEQ ID NO:1, a glutamic acid at a position corresponding to position 38 of SEQ ID NO:1, a histidine at a position corresponding to position 43 of SEQ ID NO:1, a lysine at a position corresponding to position 44 of SEQ ID NO:1, an isoleucine at apposition corresponding to position 49 of SEQ ID NO:1, a methionine at apposition corresponding to position 52 of SEQ ID NO:1, a glutamine at a position corresponding to position 69 of SEQ ID NO:1, a serine at a position corresponding to position 70 of SEQ ID NO: 1, a valine at a position corresponding to position 85 of SEQ ID NO:1, an aspartic acid at a position corresponding to position 89 of SEQ ID NO:1, an arginine at a position corresponding to position 97 of SEQ ID NO:1, and a lysine at a position corresponding to position 107 of SEQ ID NO:1.

In certain aspects, the amino acid sequence may comprise at least 10 amino acid residues, at least 10 amino acid residues, at least 11 amino acid residues, at least 12 amino acid residues, or at least 13 amino acid residues selected from the group consisting of A11, A15, K22, E38, H43, K44, 149, M52, Q69, S70, V85, D89, R97, and K107, of SEQ ID NO:15. In certain aspects, the modified 6C domain may comprise A11, A15, K22, E38, H43, K44, 149, M52, Q69, S70, V85, D89, R97, and K107, of SEQ ID NO:15. In certain aspects, the amino acid sequence may comprise one or more amino acids selected from the group consisting of an alanine at position 11, an alanine at position 15, a lysine at position 22, a glutamic acid at position 38, a histidine at position 43, a lysine at position 44, an isoleucine at position 49, a methionine at position 52, a glutamine at position 69, a serine at position 70, a valine at position 85, an aspartic acid at position 89, an arginine at position 97, and a lysine at position 107. In certain aspects, the amino acid sequence may comprise an alanine at position 11, an alanine at position 15, a lysine at position 22, a glutamic acid at position 38, a histidine at position 43, a lysine at position 44, an isoleucine at position 49, a methionine at position 52, a glutamine at position 69, a serine at position 70, a valine at position 85, an aspartic acid at position 89, an arginine at position 97, and a lysine at position 107. In certain aspects, the amino acid sequence may comprise SEQ ID NO:15.

### Immunogen 17-5

In certain aspects, amino acid sequence may differ from SEQ ID NO: 1 at one or more amino acid positions selected from the group consisting of an amino acid position corresponding to position 11 of SEQ ID NO:1, an amino acid position corresponding to position 15 of SEQ ID NO:1, an amino acid position corresponding to position 44 of SEQ ID NO:1, an amino acid position corresponding to position 52 of SEQ ID NO:1, an amino acid position corresponding to position 69 of SEQ ID NO:1, and an amino acid position corresponding to position 107 of SEQ ID NO: 1.

In certain aspects, the amino acid sequence may comprise one or more amino acids selected from the group consisting of an alanine at a position corresponding to position 11 of SEQ ID NO:1, an alanine at a position corresponding to position 15 of SEQ ID NO:1, a lysine at a position corresponding to position 44 of SEQ ID NO: 1, a methionine at apposition corresponding to position 52 of SEQ ID NO:1, a glutamine at a position corresponding to position 69 of SEQ ID NO:1, and a lysine at a position corresponding to position 107 of SEQ ID NO:1.

In certain aspects, the amino acid sequence may comprise at least 10 amino acid residues, at least 3 amino acid residues, at least 4 amino acid residues, or at least 5 amino acid residues, selected from the group consisting of A11, A15, K44, M52, Q69, and K107, of SEQ ID NO: 16. In certain aspects, the modified 6C domain may comprise A11, A15, K44, M52, Q69, and K107, of SEQ ID NO: 16. In certain aspects, the amino acid sequence may comprise one or more amino acids selected from the group consisting of an alanine at position 11, an alanine at position 15, a lysine at position 44, a methionine at position 52, a glutamine at position 69, and a lysine at position 107. In certain aspects, the amino acid sequence may comprise an alanine at position 11, an alanine at position 15, a lysine at position 44, a methionine at position 52, a glutamine at position 69, and a lysine at position 107. In certain aspects, the amino acid sequence may comprise SEQ ID NO:16.

### Immunogen 17-6

In certain aspects, amino acid sequence may differ from SEQ ID NO: 1 at one or more amino acid positions selected from the group consisting of an amino acid position corresponding to position 11 of SEQ ID NO:1, an amino acid position corresponding to position 15 of SEQ ID NO:1, an amino acid position corresponding to position 52 of SEQ ID NO:1, and an amino acid position corresponding to position 107 of SEQ ID NO:1.

In certain aspects, the amino acid sequence may comprise one or more amino acids selected from the group consisting of an alanine at a position corresponding to position 11 of SEQ ID NO:1, an alanine at a position corresponding to position 15 of SEQ ID NO:1, a methionine at apposition corresponding to position 52 of SEQ ID NO:1, and a lysine at a position corresponding to position 107 of SEQ ID NO:1.

In certain aspects, the amino acid sequence may comprise at least 10 amino acid residues, at least 2 amino acid residues, or at least 3 amino acid residues, selected from the group consisting of A11, A15, M52, and K107, of SEQ ID NO: 17. In certain aspects, the modified 6C domain may comprise A11, A15, M52, and K107, of SEQ ID NO:17. In certain aspects, the amino acid sequence may comprise one or more amino acids selected from the group consisting of an alanine at position 11, an alanine at position 15, a methionine at position 52, and a lysine at position 107. In certain aspects, the amino acid sequence may comprise SEQ ID NO:17.

One aspect of the disclosure is a fusion protein comprising at least two immunogens, 6C domains and/or CSP proteins joined together, thereby forming the fusion protein. In certain aspects, the fusion protein may comprise a modified 6C domain of the disclosure joined to a modified CSP of the disclosure. Any modified 6C domain of the disclosure may be joined to a modified CSP of the disclosure. In certain aspects, the carboxy-terminal end of the modified 6C domain may be joined to the amino terminal end of the modified CSP protein. In certain aspects, the carboxy-terminal end of the modified CSP may be joined to the amino terminal end of the modified 6C domain.

### Immunogen 17-4

In certain aspects, the modified 6C domain may comprise an amino acid sequence at least 90% identical, or at least 94% identical to SEQ ID NO:1, wherein the modified 6C domain comprises mutations at one or more amino acid positions selected from the group consisting of an amino acid position corresponding to position 11 of SEQ ID NO:1, an amino acid position corresponding to position 15 of SEQ ID NO:1, an amino acid position corresponding to position 22 of SEQ ID NO:1, an amino acid position corresponding to position 44 of SEQ ID NO:1, an amino acid position corresponding to position 52 of SEQ ID NO:1, an amino acid position corresponding to position 69 of SEQ ID NO:1, and an amino acid position corresponding to position 107 of SEQ ID NO:1. In certain aspects, the modified 6C domain may comprise one or more amino acid positions selected from the group consisting of an alanine at a position corresponding to position 11 of SEQ ID NO:1, an alanine at a position corresponding to position 15 of SEQ ID NO:1, a lysine at a position corresponding to position 22 of SEQ ID NO:1, a lysine at a position corresponding to position 44 of SEQ ID NO:1, a methionine at a position corresponding to position 52 of SEQ ID NO:1, a glutamine at a position corresponding to position 69 of SEQ ID NO:1, and a lysine at a position corresponding to position 107 of SEQ ID NO:1 In certain aspects, the modified 6C domain may comprise one or more amino acid residues selected from the group consisting of A11, A15, K22, K44, M52, Q69, and K107, of SEQ ID NO:5. In certain aspects, the modified 6C domain may comprise A11, A15, K22, K44, M52, Q69, and K107, of SEQ ID NO:5. In certain aspects, the modified 6C domain may comprise one or more amino acid residues selected from the group consisting of an alanine position 11, an alanine at position 15, a lysine at position 22, a lysine at position 44, a methionine at position 52, a glutamine at position 69, and a lysine at position 107. In certain aspects, the modified 6C domain may comprise an alanine position 11, an alanine at position 15, a lysine at position 22, a lysine at position 44, a methionine at position 52, a glutamine at position 69, and a lysine at position 107. In certain aspects, the modified 6C domain may comprise SEQ ID NO:5.

In certain aspects, the modified CSP protein may comprise a CSP junction region, or a portion thereof, preferably comprising the nAB CIS43 epitope, joined to a NANP repeat region joined to a portion of the conserved C-terminal region, wherein the NANP repeat region comprises, consists of, or consists essentially of, 3-7 consecutive NANP amino acid sequence repeats, and wherein the modified CSP lacks the N-terminal region of a wild-type (wt) CSP protein. In certain aspects, the junction region, or a portion thereof, may comprise an amino acid sequence corresponding to amino acids 120-131 of SEQ ID NO:31. In certain aspects, the junction region, or a portion thereof, may comprise, consist of, or consist essentially of, amino acid sequence at least 83%, or 100%, identical to SEQ ID NO:37. In certain aspects, the NANP repeat region may comprise five consecutive NANP amino acid sequences. In certain aspects, the portion of the conserved-C terminal region may comprise, consist of, or consist essentially of, an amino acid sequence corresponding to amino acids 313-386 of SEQ ID NO: 27. In certain aspects, the junction region, or a portion thereof, may comprise, consist of, or consist of, amino acid sequence at least 90% %, at least 95%, at least 97%, or 100%, identical to SEQ ID NO:38. In certain aspects, the modified CSP may lack an amino acid sequence corresponding to amino acids 1-135, or 18-135, of SEQ ID NO:27. In certain aspects, a modified CSP of the disclosure may comprise, consist of, or consist of, a junction region, or a portion thereof, joined a NANP repeat region, which is joined to a conserved C-terminal region, wherein the junction region, or a portion thereof, comprises, consists of, or consist essentially of, an amino acid sequence at least 83%, or 100%, identical to SEQ ID NO:37, wherein the NANP repeat region consists, or consists essentially of, 3-7 consecutive NANP amino acid sequences, and wherein the conserved-C-terminal region may comprise, consist of, or consist essentially of, an amino acid sequence at least 90% %, at least 95%, at least 97%, or 100%, identical to SEQ ID NO: 38. In certain aspects, the modified CSP may lack an amino acid sequence corresponding to amino acids 1-135, or 18-135, of SEQ ID NO:27.

In certain aspects, the modified CSP of the disclosure may comprise, consist of, or consist essentially of, an amino acid sequence at least at least 90% %, at least 95%, at least 97%, or at least 99% identical to SEQ ID NO:32, wherein the modified CSP comprises a first amino acid sequence at least 83%, or 100% identical to SEQ ID NO:37 joined to a second amino acid sequence consisting of, or consisting essentially of, three, four, five, six or seven consecutive NANP sequences, wherein the second amino acid sequence is joined to a third amino acid sequence comprising, consisting of, or consisting essentially of, an amino acid sequence at least 90% %, at least 95%, at least 97%, or 100%, identical to SEQ ID NO:38. In certain aspects, a modified CSP of the disclosure may comprise, consist of, or consist essentially of SEQ ID NO:32.

In certain aspects, the carboxy terminal end of the modified 6C domain may be joined to the amino terminal end of the modified CSP. In certain aspects, the carboxy terminal end of the modified CSP domain may be joined to the amino terminal end of the modified 6C domain. In certain aspects, the fusion protein may comprise an amino acid sequence at least an amino acid sequence a least 90%, at least 95%, or at least 97%, identical to SEQ ID NO:33 or 34, wherein the fusion protein comprises one or more amino acid positions selected from the group consisting of an alanine at a position corresponding to position 11 of SEQ ID NO:1, an alanine at a position corresponding to position 15 of SEQ ID NO:1, a lysine at a position corresponding to position 22 of SEQ ID NO:1, a lysine at a position corresponding to position 44 of SEQ ID NO:1, a methionine at a position corresponding to position 52 of SEQ ID NO:1, a glutamine at a position corresponding to position 69 of SEQ ID NO:1, and a lysine at a position corresponding to position 107 of SEQ ID NO:1 In certain aspects, the modified 6C domain may comprise one or more amino acid residues selected from the group consisting of A11, A15, K22, K44, M52, Q69, and K107, of SEQ ID NO:5. In certain aspects, the fusion protein may comprise an amino acid sequence at least an amino acid sequence a least 90%, at least 95%, or at least 97%, identical to SEQ ID NO:33 or 34, wherein the fusion protein comprises an alanine at a position corresponding to position 11 of SEQ ID NO:1, an alanine at a position corresponding to position 15 of SEQ ID NO:1, a lysine at a position corresponding to position 22 of SEQ ID NO:1, a lysine at a position corresponding to position 44 of SEQ ID NO: 1, a methionine at a position corresponding to position 52 of SEQ ID NO:1, a glutamine at a position corresponding to position 69 of SEQ ID NO:1, and a lysine at a position corresponding to position 107 of SEQ ID NO:1. In certain aspects, the fusion protein may comprise SQEQ ID NO: 5 joined to SEQ ID NO:32. In certain aspects, the fusion protein may comprise SEQ ID NO:33 or 34.

One aspect of the disclosure is a fusion protein comprising a self-assembling subunit protein joined to a modified 6C domain of the disclosure or an isolated protein of the disclosure. As used herein, a fusion protein is a recombinant protein containing amino acid sequence from at least two unrelated proteins that have been joined together, via a peptide bond, to make a single protein. The unrelated amino acid sequences can be joined directly to each other, or they can be joined using a linker sequence. As used herein, proteins are unrelated, if their amino acid sequences are not normally found joined together via a peptide bond in their natural environment(s) (e.g., inside a cell). For example, the amino acid sequences of monomeric subunits that make up ferritin, and the amino acid sequences of P48/45 are not normally found joined together via a peptide bond.

As used herein, a self-assembling (SA) subunit protein of the present invention is a full length, monomeric polypeptide, or at least 50, or at least 100 contiguous amino acids from any portion thereof, which can direct self-assembly of monomeric self-assembling subunit proteins into a nanoparticle. Examples of self-assembly proteins of the present invention include, but are not limited to, ferritin, encapsulin, sulfur oxygenase reductase (SOR), lumazine synthase (LS) and pyruvate dehydrogenase complex (PDC) dihydrolipoamide acetyltransferase (E2).

Thus, one embodiment of the present invention is fusion protein comprising a self-assembling subunit protein selected from the group consisting of ferritin, encapsulin, sulfur oxygenase reductase, lumazine synthase and dihydrolipoamide acetyltransferase (E2), joined to a modified 6C domain or an isolated protein disclosed herein, wherein the fusion protein is capable of self-assembling into nanoparticles.

In one embodiment, the self-assembly protein is ferritin. Ferritin forms a spherical protein found in all animals, bacteria, and plants, that acts primarily to control the rate and location of polynuclear Fe(III)₂O₃ formation through the transportation of hydrated iron ions and protons to and from a mineralized core. The spherical form of ferritin is made up of monomeric subunit proteins (also referred to as monomeric ferritin subunits), which are polypeptides having a molecule weight of approximately 17-20 kDa. Examples of the sequences of suitable monomeric ferritin subunits include, but are not limited to, SEQ ID NO:22-24 and SEQ ID NO:40.

According to the present invention, a monomeric ferritin subunit of the present invention is a full length, single polypeptide of a ferritin protein, or at least 50, at least 100, or at least 150 contiguous amino acids from any portion thereof, which can direct self-assembly of monomeric ferritin subunits into the spherical form of the protein. In certain aspects, the monomeric ferritin subunit may comprise an amino acid sequence at least 80%, at least 85%, at least 90%, at least 95%, at least 97%, or 100%, identical to a known ferritin protein, such as SEQ ID NO:22, 23, 24 or 40, wherein the monomeric ferritin subunit self-assembles into nanoparticles. Amino acid sequences from monomeric ferritin subunits of any known ferritin protein can be used to produce fusion proteins of the present invention, so long as the monomeric ferritin subunit is capable of directing self-assembly of the fusion protein into a nanoparticle displaying a modified 6C domain or an isolated protein of the disclosure on its surface. In certain aspects, the monomeric subunit is from a ferritin protein selected from the group consisting of a bacterial ferritin protein, a plant ferritin protein, an algal ferritin protein, an insect ferritin protein, a fungal ferritin protein and a mammalian ferritin protein. In certain aspects, the ferritin protein is from Helicobacter pylori. In one embodiment, the ferritin protein is from E. coli. In certain aspects, the ferritin protein is bullfrog ferritin. In certain aspects, the ferritin protein may comprise amino acid sequences from one or more ferritin proteins selected from the group consisting of H. pylori ferritin, E. coli ferritin and bullfrog ferritin (e.g., UniProt entry P07797). Amino acid sequences from representative ferritin proteins of the present invention are disclosed herein as SEQ ID NO: 22 (H. pylori ferritin), SEQ ID NO:23 (H. pylori ferritin), SEQ ID NO:24 (H. pylori ferritin-bullfrog ferritin fusion), and SEQ ID NO:40.

In certain aspects, only a portion of a ferritin protein may be fused to a modified 6C domain of the disclosure, an isolated protein of the disclosure, an immunogen of the disclosure, a modified CSP of the disclosure, or a fusion protein of the disclosure. Such portions include, but are not limited to, amino acid residues 2-9 of bullfrog ferritin, amino acid residues 3-167, or amino acid residues 5-167 of H. pylori ferritin. In certain aspects, mutations made be introduced into ferritin proteins to prevent glycosylation of the fusion protein or to preserve intra-molecular bridges. For example, N8 of bullfrog ferritin may be altered to prevent glycosylation. Similarly, 6R of H. pylori ferritin may be altered to prevent glycosylation. As a further example, 17 of H. pylori ferritin may be mutated to preserve salt bridges.

In certain aspects, the self-assembly protein is encapsulin. According to the present invention, a monomeric encapsulin subunit of the present invention is a full length, single polypeptide of an encapsulin protein, or at least 50, or at least 100 contiguous amino acids from any portion thereof, which can direct self-assembly of monomeric encapsulin subunits into a nanoparticle. Amino acid sequences from monomeric encapsulin subunits of any known encapsulin protein can be used to produce fusion proteins of the present invention, so long as the monomeric encapsulin subunit can direct self-assembly of the fusion protein into a nanoparticle displaying a modified 6C domain or an isolated protein disclosed herein on its surface.

In certain aspects, the self-assembly protein is artificially designed Salmonella enteritis 03-33 subunit protein. A monomeric 03-33 subunit is a full length, single polypeptide of an 03-33 protein, or any portion thereof, which can direct self-assembly of monomeric 03-33 subunits into a nanoparticle. Amino acid sequences from monomeric 03-33 subunits of any known 03-33 protein can be used to produce fusion proteins of the disclosure, so long as the monomeric 03-33 subunit can direct self-assembly of the fusion protein into a nanoparticle displaying a modified 6C domain or an isolated protein disclosed herein on its surface.

In certain aspects, the self-assembly protein is sulfur oxygenase reductase (SOR). According to the present invention, a monomeric SOR subunit of the present invention is a full length, single polypeptide of an SOR protein, or at least 100 contiguous amino acids from any portion thereof, which can direct self-assembly of monomeric SOR subunits into a nanoparticle. Amino acid sequences from monomeric SOR subunits of any known SOR protein can be used to produce fusion proteins of the present invention, so long as the monomeric SOR subunit can direct self-assembly of the fusion protein into a nanoparticle displaying a modified 6C domain or an isolated protein disclosed herein on its surface.

In one embodiment, the self-assembly protein is lumazine synthase (LS). According to the present invention, a monomeric LS subunit of the present invention is a full length, single polypeptide of an LS protein, or at least 100 contiguous amino acids from any portion thereof, which can direct self-assembly of monomeric LS subunits into a nanoparticle. Amino acid sequences from monomeric LS subunits of any known LS protein can be used to produce fusion proteins of the present invention, so long as the monomeric LS subunit can direct self-assembly of the fusion protein into a nanoparticle displaying a modified 6C domain or an isolated protein disclosed on its surface.

In one embodiment, the self-assembly protein is pyruvate dehydrogenase complex (PDC) dihydrolipoamide acetyltransferase (E2p). According to the present invention, a monomeric E2p subunit of the present invention is a full length, single polypeptide of an E2p protein, or at least 100 contiguous amino acids from any portion thereof, which can direct self-assembly of monomeric E2p subunits into a nanoparticle. Amino acid sequences from monomeric E2p subunits of any known E2p protein can be used to produce fusion proteins of the present invention, so long as the monomeric E2p subunit can direct self-assembly of the fusion protein into a nanoparticle displaying a modified 6C domain or an isolated protein disclosed on its surface.

Fusion proteins of the present disclosure need not comprise the full-length sequence of a monomeric subunit polypeptide of a self-assembly (SA) protein. Portions, or regions, of the SA monomeric subunit protein can be utilized so long as the portion may comprise at least 100 contiguous amino acids from an amino acid sequence that directs self-assembly of the 6C-SA fusion protein into a nanoparticle. Examples of such portions include between amino acids 3 and 167 or between amino acids 5 and 167 of the Helicobacter pylori ferritin protein. More specific regions of the ferritin protein are described in Zhang, Y. Self-Assembly in the Ferritin Nano-Cage Protein Super Family. 2011, Int. J. Mol. Sci., 12, 5406-5421.

In certain aspects, it may be useful to engineer mutations into the amino acid sequences of self-assembly proteins of the present invention. For example, it may be useful to alter sites such as enzyme recognition sites or glycosylation sites in the monomeric ferritin subunit, the trimerization domain, or linker sequences, in order to give the fusion protein beneficial properties (e.g., stability, solubility, half-life, mask portions of the protein from immune surveillance). For example, it is known that the monomeric subunit of ferritin is not glycosylated naturally. However, it can be glycosylated if it is expressed as a secreted protein in mammalian or yeast cells. Thus, in certain aspects, potential N-linked glycosylation sites in the amino acid sequences from the monomeric ferritin subunit are mutated so that the mutated ferritin subunit sequences are no longer glycosylated at the mutated site.

In certain aspects, joining of the self-assembling subunit and a modified 6C domain of the disclosure, an isolated protein of the disclosure, an immunogen of the disclosure, a modified CSP of the disclosure, or a fusion protein of the disclosure, may be done such that the sequences are directly linked. In certain aspects, it may be necessary to employ linkers (also referred to as a spacer sequences) between the self-assembling subunit and the modified 6C domain or isolated protein of the disclosure so that the so that they are in the proper orientation. More specifically, linker sequence can be inserted so that the 6C domain or isolated protein of the disclosure is positioned in such a way to maintain the ability to elicit an immune response against the 85RF45.1 epitope. Linker sequences of the present disclosure comprise amino acids, preferably those having small side chains and/or those which are not charged. Such amino acids are less likely to interfere with proper folding and activity of the fusion protein. Accordingly, preferred amino acids to use in linker sequences, either alone or in combination are serine, glycine and alanine. Examples of such linker sequences include, but are not limited to, a single serine or glycine, dipeptides thereof, SGG, GSGG (SEQ ID NO:26), GSG, GG, GTGSGGGG (SEQ ID NO:25), variations thereof and iterations thereof. Amino acids can be added, subtracted, or substituted as needed. It is within the ability of a person killed in the art to determine appropriate linker sequences for fusion proteins of the present disclosure.

In certain aspects, a fusion protein may comprise an immunogen of the disclosure fused to a ferritin protein, or fragments thereof, wherein the ferritin protein, or fragment thereof, directs assembly of the fusion protein into nanoparticles. In certain aspects, the immunogen may comprise a modified C6 domain comprising an amino acid sequence having at least about 70%, at least about 72%, at least about 74%, at least about 75%, at least about 80%, at least about 85%, at least about 90%, at least about 92%, at least about 94%, at least about 96%, at least about 98%, or at least about 99%, homology over the entire length of a 6C domain from a Plasmodium 48/45 protein, wherein the amino acid sequence comprises one or more mutations relative to the wt Plasmodium 6C amino acid sequence, that increase the stability of the immunogen relative to the stability of a second immunogen comprising the amino acid sequence of the wt Plasmodium 6C domain. In certain aspects, the immunogen may comprise a 85RF45.1 epitope.

In certain aspects, the immunogen may comprise one or more amino acids selected from the group consisting of a serine at a position corresponding to position 3 of SEQ ID NO:1, an alanine at a position corresponding to position 6 of SEQ ID NO: 1, an alanine at a position corresponding to position 11 of SEQ ID NO: 1, a lysine at a position corresponding to position 14 of SEQ ID NO: 1, an asparagine at a position corresponding to position 15 of SEQ ID NO: 1, a tyrosine at a position corresponding to position 18 of SEQ ID NO:1, a lysine at a position corresponding to position 19 of SEQ ID NO: 1, an alanine at a position corresponding to position 21 of SEQ ID NO: 1, a lysine at a position corresponding to position 22 of SEQ ID NO: 1, a glutamine at a position corresponding to position 28 of SEQ ID NO:1, a lysine at a position corresponding to position 33 of SEQ ID NO:1, a valine at a position corresponding to position 35 of SEQ ID NO: 1, an as arginine at a position corresponding to position 36 of SEQ ID NO:1, a glutamic acid at a position corresponding to position 38 of SEQ ID NO:1, a histidine at a position corresponding to position 43 of SEQ ID NO:1, a glutamic acid at a position corresponding to position 45 of SEQ ID NO:1, an isoleucine at a position corresponding to position 49 of SEQ ID NO:1, histidine at a position corresponding to position 68 of SEQ ID NO:1, an asparagine at a position corresponding to position 69 of SEQ ID NO:1, a serine at a position corresponding to position 70 of SEQ ID NO: 1, an alanine at a position corresponding to position 71 of SEQ ID NO:1, a glutamic acid at a position corresponding to position 83 of SEQ ID NO:1, as aspartic acid at a position corresponding to position 84 of SEQ ID NO:1, a leucine at a position corresponding to position 86 of SEQ ID NO:1, an alanine at a position corresponding to position 89 of SEQ ID NO:1, an asparagine at a position corresponding to position 90 of SEQ ID NO:1, an arginine at a position corresponding to position 97 of SEQ ID NO: 1, a methionine at a position corresponding to position 105 of SEQ ID NO:1, an alanine at a position corresponding to position 107 of SEQ ID NO:1, an isoleucine at a position corresponding to position 109 of SEQ ID NO:1, a glutamic acid at a position corresponding to position 114 of SEQ ID NO:1, a proline at a position corresponding to position 115 of SEQ ID NO: 1, a phenylalanine at a position corresponding to position 134 of SEQ ID NO:1, and a serine at a position corresponding to position 138 of SEQ ID NO: 1.

In certain aspects, the fusion protein may comprise an amino acid sequence at least 85%, at least 90%, at least 95%, or at least 97% identical to SEQ ID NO: 18, wherein the fusion protein may comprise one or more amino acids selected from the group consisting of a serine at a position corresponding to position 3 of SEQ ID NO: 1, an alanine at a position corresponding to position 6 of SEQ ID NO:1, an alanine at a position corresponding to position 11 of SEQ ID NO:1, a lysine at a position corresponding to position 14 of SEQ ID NO:1, an asparagine at a position corresponding to position 15 of SEQ ID NO:1, a tyrosine at a position corresponding to position 18 of SEQ ID NO:1, a lysine at a position corresponding to position 19 of SEQ ID NO:1, an alanine at a position corresponding to position 21 of SEQ ID NO:1, a lysine at a position corresponding to position 22 of SEQ ID NO:1, a glutamine at a position corresponding to position 28 of SEQ ID NO:1, a lysine at a position corresponding to position 33 of SEQ ID NO: 1, a valine at a position corresponding to position 35 of SEQ ID NO:1, an as arginine at a position corresponding to position 36 of SEQ ID NO:1, a glutamic acid at a position corresponding to position 38 of SEQ ID NO:1, a histidine at a position corresponding to position 43 of SEQ ID NO:1, a glutamic acid at a position corresponding to position 45 of SEQ ID NO:1, an isoleucine at a position corresponding to position 49 of SEQ ID NO:1, histidine at a position corresponding to position 68 of SEQ ID NO:1, an asparagine at a position corresponding to position 69 of SEQ ID NO:1, a serine at a position corresponding to position 70 of SEQ ID NO:1, an alanine at a position corresponding to position 71 of SEQ ID NO:1, a glutamic acid at a position corresponding to position 83 of SEQ ID NO:1, as aspartic acid at a position corresponding to position 84 of SEQ ID NO:1, a leucine at a position corresponding to position 86 of SEQ ID NO:1, an alanine at a position corresponding to position 89 of SEQ ID NO:1, an asparagine at a position corresponding to position 90 of SEQ ID NO:1, an arginine at a position corresponding to position 97 of SEQ ID NO:1, a methionine at a position corresponding to position 105 of SEQ ID NO:1, an alanine at a position corresponding to position 107 of SEQ ID NO:1, an isoleucine at a position corresponding to position 109 of SEQ ID NO:1, a glutamic acid at a position corresponding to position 114 of SEQ ID NO:1, a proline at a position corresponding to position 115 of SEQ ID NO:1, a phenylalanine at a position corresponding to position 134 of SEQ ID NO:1, and a serine at a position corresponding to position 138 of SEQ ID NO: 1. In certain aspects, the fusion protein may comprise SEQ ID NO:18.

In certain aspects, the immunogen may comprise one or more amino acids selected from the group consisting of an alanine at a position corresponding to position 6 of SEQ ID NO:1, an alanine at a position corresponding to position 11 of SEQ ID NO:1, a lysine at a position corresponding to position 14 of SEQ ID NO:1, an alanine at a position corresponding to position 15 of SEQ ID NO:1, a tyrosine at a position corresponding to position 18 of SEQ ID NO:1, a lysine at a position corresponding to position 19 of SEQ ID NO:1, a lysine at a position corresponding to position 22 of SEQ ID NO:1, a lysine at a position corresponding to position 33 of SEQ ID NO: 1, an arginine at a position corresponding to position 36 of SEQ ID NO:1, a glutamic acid at a position corresponding to position 38 of SEQ ID NO:1, a histidine at a position corresponding to position 43 of SEQ ID NO:1, an isoleucine at a position corresponding to position 49 of SEQ ID NO:1, an asparagine at a position corresponding to position 69 of SEQ ID NO:1, a serine at a position corresponding to position 70 of SEQ ID NO:1, an alanine at a position corresponding to position 71 of SEQ ID NO:1, a glutamic acid at a position corresponding to position 83 of SEQ ID NO:1, a leucine at a position corresponding to position 86 of SEQ ID NO:1, an alanine at a position corresponding to position 89 of SEQ ID NO:1, an arginine at a position corresponding to position 97 of SEQ ID NO:1, a methionine at a position corresponding to position 105 of SEQ ID NO:1, an alanine at a position corresponding to position 107 of SEQ ID NO:1, a proline at a position corresponding to position 115 of SEQ ID NO:1, and a phenylalanine at a position corresponding to position 134 of SEQ ID NO:1.

In certain aspects, the fusion protein may comprise an amino acid sequence at least 85%, at least 90%, at least 95%, or at least 97% identical to SEQ ID NO: 19, wherein the fusion protein may comprise one or more amino acids selected from the group consisting of an alanine at a position corresponding to position 6 of SEQ ID NO: 1, an alanine at a position corresponding to position 11 of SEQ ID NO:1, a lysine at a position corresponding to position 14 of SEQ ID NO:1, an alanine at a position corresponding to position 15 of SEQ ID NO:1, a tyrosine at a position corresponding to position 18 of SEQ ID NO:1, a lysine at a position corresponding to position 19 of SEQ ID NO: 1, a lysine at a position corresponding to position 22 of SEQ ID NO:1, a lysine at a position corresponding to position 33 of SEQ ID NO:1, an arginine at a position corresponding to position 36 of SEQ ID NO:1, a glutamic acid at a position corresponding to position 38 of SEQ ID NO:1, a histidine at a position corresponding to position 43 of SEQ ID NO:1, an isoleucine at a position corresponding to position 49 of SEQ ID NO:1, an asparagine at a position corresponding to position 69 of SEQ ID NO: 1, a serine at a position corresponding to position 70 of SEQ ID NO:1, an alanine at a position corresponding to position 71 of SEQ ID NO:1, a glutamic acid at a position corresponding to position 83 of SEQ ID NO:1, a leucine at a position corresponding to position 86 of SEQ ID NO:1, an alanine at a position corresponding to position 89 of SEQ ID NO:1, an arginine at a position corresponding to position 97 of SEQ ID NO:1, a methionine at a position corresponding to position 105 of SEQ ID NO:1, an alanine at a position corresponding to position 107 of SEQ ID NO:1, a proline at a position corresponding to position 115 of SEQ ID NO:1, and a phenylalanine at a position corresponding to position 134 of SEQ ID NO:1. In certain aspects, the fusion protein may comprise SEQ ID NO:19.

In certain aspects, the immunogen may comprise one or more amino acids selected from the group consisting of a serine at a position corresponding to position 3 of SEQ ID NO:1, an alanine at a position corresponding to position 11 of SEQ ID NO: 1, a lysine at a position corresponding to position 14 of SEQ ID NO:1, an asparagine at a position corresponding to position 15 of SEQ ID NO: 1, a lysine at a position corresponding to position 19 of SEQ ID NO:1, an alanine at a position corresponding to position 21 of SEQ ID NO:1, a lysine at a position corresponding to position 22 of SEQ ID NO:1, a glutamine at a position corresponding to position 28 of SEQ ID NO:1, a lysine at a position corresponding to position 33 of SEQ ID NO:1, a valine at a position corresponding to position 35 of SEQ ID NO:1, an arginine at a position corresponding to position 36 of SEQ ID NO:1, a glutamic acid at a position corresponding to position 38 of SEQ ID NO:1, a glutamic acid at a position corresponding to position 40 of SEQ ID NO: 1, a histidine at a position corresponding to position 43 of SEQ ID NO:1, a lysine at a position corresponding to position 44 of SEQ ID NO: 1, a tyrosine at a position corresponding to position 45 of SEQ ID NO: 1, an isoleucine at a position corresponding to position 49 of SEQ ID NO:4, a methionine at a position corresponding to position 52 of SEQ ID NO:1, a histidine at a position corresponding to position 68 of SEQ ID NO:1, a glutamine at a position corresponding to position 69 of SEQ ID NO: 1, a serine at a position corresponding to position 70 of SEQ ID NO: 1, as aspartic acid at a position corresponding to position 84 of SEQ ID NO:1, a valine at a position corresponding to position 85 of SEQ ID NO:1, an aspartic acid at a position corresponding to position 89 of SEQ ID NO:1, an arginine at a position corresponding to position 97 of SEQ ID NO:1, a lysine at a position corresponding to position 107 of SEQ ID NO: 1, a proline at a position corresponding to position 115 of SEQ ID NO:1, a lysine at a position corresponding to position 134 of SEQ ID NO:1, and a serine at a position corresponding to position 138 of SEQ ID NO:1.

In certain aspects, the fusion protein may comprise an amino acid sequence at least 85%, at least 90%, at least 95%, or at least 97% identical to SEQ ID NO: 20, wherein the fusion protein may comprise one or more amino acids selected from the group consisting of a serine at a position corresponding to position 3 of SEQ ID NO: 1, an alanine at a position corresponding to position 11 of SEQ ID NO:1, a lysine at a position corresponding to position 14 of SEQ ID NO:1, an asparagine at a position corresponding to position 15 of SEQ ID NO:1, a lysine at a position corresponding to position 19 of SEQ ID NO:1, an alanine at a position corresponding to position 21 of SEQ ID NO:1, a lysine at a position corresponding to position 22 of SEQ ID NO:1, a glutamine at a position corresponding to position 28 of SEQ ID NO:1, a lysine at a position corresponding to position 33 of SEQ ID NO:1, a valine at a position corresponding to position 35 of SEQ ID NO:1, an arginine at a position corresponding to position 36 of SEQ ID NO:1, a glutamic acid at a position corresponding to position 38 of SEQ ID NO:1, a glutamic acid at a position corresponding to position 40 of SEQ ID NO: 1, a histidine at a position corresponding to position 43 of SEQ ID NO:1, a lysine at a position corresponding to position 44 of SEQ ID NO:1, a tyrosine at a position corresponding to position 45 of SEQ ID NO:1, an isoleucine at a position corresponding to position 49 of SEQ ID NO:4, a methionine at a position corresponding to position 52 of SEQ ID NO:1, a histidine at a position corresponding to position 68 of SEQ ID NO:1, a glutamine at a position corresponding to position 69 of SEQ ID NO:1, a serine at a position corresponding to position 70 of SEQ ID NO:1, as aspartic acid at a position corresponding to position 84 of SEQ ID NO:1, a valine at a position corresponding to position 85 of SEQ ID NO:1, an aspartic acid at a position corresponding to position 89 of SEQ ID NO:1, an arginine at a position corresponding to position 97 of SEQ ID NO: 1, a lysine at a position corresponding to position 107 of SEQ ID NO:1, a proline at a position corresponding to position 115 of SEQ ID NO:1, a lysine at a position corresponding to position 134 of SEQ ID NO:1, and a serine at a position corresponding to position 138 of SEQ ID NO:1. In certain aspects, the fusion protein may comprise SEQ ID NO:20.

In certain aspects, the immunogen may comprise one or more amino acids selected from the group consisting of an alanine at a position corresponding to position 11 of SEQ ID NO:1, an alanine at a position corresponding to position 15 of SEQ ID NO:1, a lysine at a position corresponding to position 22 of SEQ ID NO:1, a lysine at a position corresponding to position 44 of SEQ ID NO:1, a methionine at a position corresponding to position 52 of SEQ ID NO:1, a glutamine at a position corresponding to position 69 of SEQ ID NO:1, and a lysine at a position corresponding to position 107 of SEQ ID NO:1.

In certain aspects, the fusion protein may comprise an amino acid sequence at least 85%, at least 90%, at least 95%, or at least 97% identical to SEQ ID NO: 21, wherein the fusion protein may comprise one or more amino acids selected from the group consisting of an alanine at a position corresponding to position 11 of SEQ ID NO: 1, an alanine at a position corresponding to position 15 of SEQ ID NO:1, a lysine at a position corresponding to position 22 of SEQ ID NO:1, a lysine at a position corresponding to position 44 of SEQ ID NO:1, a methionine at a position corresponding to position 52 of SEQ ID NO:1, a glutamine at a position corresponding to position 69 of SEQ ID NO:1, and a lysine at a position corresponding to position 107 of SEQ ID NO:1. In certain aspects, the fusion protein may comprise SEQ ID NO:21.

In certain aspects, the immunogen may comprise a modified CSP protein of the disclosure. In certain aspects, the modified CSP protein may comprise a CSP junction region, or a portion thereof, joined to a NANP repeat region joined to a portion of the conserved C-terminal region, wherein the NANP repeat region comprises, consists of, or consists essentially of, 3-7 consecutive NANP amino acid sequence repeats, and wherein the modified CSP lacks the N-terminal region of a wild-type (wt) CSP protein. In certain aspects, the junction region, or a portion thereof, may comprise an amino acid sequence corresponding to amino acids 136-147 of SEQ ID NO:27. In certain aspects, the junction region, or a portion thereof, may comprise, consist of, or consist essentially of, amino acid sequence at least 83%, or 100%, identical to SEQ ID NO: 37. In certain aspects, the NANP repeat region may comprise five consecutive NANP amino acid sequences. In certain aspects, the portion of the conserved-C terminal region may comprise, consist of, or consist essentially of, an amino acid sequence corresponding to amino acids 313-386 of SEQ ID NO:27. In certain aspects, the junction region, or a portion thereof, may comprise, consist of, or consist of, amino acid sequence at least 90% %, at least 95%, at least 97%, or 100%, identical to SEQ ID NO: 38. In certain aspects, the modified CSP may lack an amino acid sequence corresponding to amino acids 1-135, or 18-135, of SEQ ID NO:27. In certain aspects, a modified CSP of the disclosure may comprise, consist of, or consist of, a junction region, or a portion thereof, joined a NANP repeat region, which is joined to a conserved C-terminal region, wherein the junction region, or a portion thereof, comprises, consists of, or consist essentially of, an amino acid sequence at least 83%, or 100%, identical to SEQ ID NO: 37, wherein the NANP repeat region consists, or consists essentially of, 3-7 consecutive NANP amino acid sequences, and wherein the conserved-C-terminal region may comprise, consist of, or consist essentially of, an amino acid sequence at least 90% %, at least 95%, at least 97%, or 100%, identical to SEQ ID NO:38. In certain aspects, the modified CSP may lack an amino acid sequence corresponding to amino acids 1-135, or 18-135, of SEQ ID NO:27.

In certain aspects, the modified CSP of the disclosure may comprise, consist of, or consist essentially of, an amino acid sequence at least at least 90% %, at least 95%, at least 97%, or at least 99% identical to SEQ ID NO:32, wherein the modified CSP comprises a first amino acid sequence at least 83%, or 100% identical to SEQ ID NO:37 joined to a second amino acid sequence consisting of, or consisting essentially of, three, four, five, six or seven consecutive NANP sequences, wherein the second amino acid sequence is joined to a third amino acid sequence comprising, consisting of, or consisting essentially of, an amino acid sequence at least 90% %, at least 95%, at least 97%, or 100%, identical to SEQ ID NO:38. In certain aspects, a modified CSP of the disclosure may comprise, consist of, or consist essentially of SEQ ID NO:32.

In certain aspects, fusion proteins of the disclosure may comprise SA monomeric subunit protein joined to multiple immunogens. Such fusion proteins may provide benefits not provided by current malaria vaccines. For example, the modified CSP elicits an immune response that protects against infection of the liver, whereas the modified C6 domain elicits an immune response against transmission of Plasmodium. Consequently, a vaccine comprising both a modified CSP and a modified C6 domain will elicit an immune response that inhibits the Plasmodium life cycle at two separate stages, namely reproduction and transmission of Plasmodium. Thus, in certain aspect, the fusion protein may comprise a SA monomeric subunit protein joined to a modified CSP of the disclosure joined, which is joined to a modified 6C domain of the disclosure. Alternatively, the fusion protein may comprise a SA monomeric subunit protein joined to a modified 6C domain of the disclosure, which is joined to a modified 6C domain of the disclosure. In certain aspects, the modified CSP and the modified 6C domain may be linked directly to one another, or they may be joined by a linker, examples of which have been disclosed herein. In certain aspects, the modified 6C domain may comprise an amino acid sequence at least 85%, at least 90%, at least 95%, or at least 97% identical to SEQ ID NO:21, wherein the fusion protein may comprise one or more amino acids selected from the group consisting of an alanine at a position corresponding to position 11 of SEQ ID NO:1, an alanine at a position corresponding to position 15 of SEQ ID NO:1, a lysine at a position corresponding to position 22 of SEQ ID NO: 1, a lysine at a position corresponding to position 44 of SEQ ID NO:1, a methionine at a position corresponding to position 52 of SEQ ID NO:1, a glutamine at a position corresponding to position 69 of SEQ ID NO:1, and a lysine at a position corresponding to position 107 of SEQ ID NO:1. In certain aspects, the modified 6C domain protein may comprise SEQ ID NO:21. In certain aspects, the modified CSP protein may comprise a CSP junction region, or a portion thereof, joined to a NANP repeat region joined to a portion of the conserved C-terminal region, wherein the NANP repeat region comprises, consists of, or consists essentially of, 3-7 consecutive NANP amino acid sequence repeats, and wherein the modified CSP lacks the N-terminal region of a wild-type (wt) CSP protein. In certain aspects, the junction region, or a portion thereof, may comprise an amino acid sequence corresponding to amino acids 136-147 of SEQ ID NO:27. In certain aspects, the junction region, or a portion thereof, may comprise, consist of, or consist essentially of, amino acid sequence at least 83%, or 100%, identical to SEQ ID NO:37. In certain aspects, the NANP repeat region may comprise five consecutive NANP amino acid sequences. In certain aspects, the portion of the conserved-C terminal region may comprise, consist of, or consist essentially of, an amino acid sequence corresponding to amino acids 313-386 of SEQ ID NO:27. In certain aspects, the junction region, or a portion thereof, may comprise, consist of, or consist of, amino acid sequence at least 90% %, at least 95%, at least 97%, or 100%, identical to SEQ ID NO:38. In certain aspects, the modified CSP may lack an amino acid sequence corresponding to amino acids 1-135, or 18-135, of SEQ ID NO:27. In certain aspects, a modified CSP of the disclosure may comprise, consist of, or consist of, a junction region, or a portion thereof, joined a NANP repeat region, which is joined to a conserved C-terminal region, wherein the junction region, or a portion thereof, comprises, consists of, or consist essentially of, an amino acid sequence at least 83%, or 100%, identical to SEQ ID NO: 37, wherein the NANP repeat region consists, or consists essentially of, 3-7 consecutive NANP amino acid sequences, and wherein the conserved-C-terminal region may comprise, consist of, or consist essentially of, an amino acid sequence at least 90% %, at least 95%, at least 97%, or 100%, identical to SEQ ID NO:38. In certain aspects, the modified CSP may lack an amino acid sequence corresponding to amino acids 1-135, or 18-135, of SEQ ID NO:27. In certain aspects, the modified CSP of the disclosure may comprise, consist of, or consist essentially of, an amino acid sequence at least at least 90% %, at least 95%, at least 97%, or at least 99% identical to SEQ ID NO: 32, wherein the modified CSP comprises a first amino acid sequence at least 83%, or 100% identical to SEQ ID NO:37 joined to a second amino acid sequence consisting of, or consisting essentially of, three, four, five, six or seven consecutive NANP sequences, wherein the second amino acid sequence is joined to a third amino acid sequence comprising, consisting of, or consisting essentially of, an amino acid sequence at least 90% %, at least 95%, at least 97%, or 100%, identical to SEQ ID NO:38. In certain aspects, a modified CSP of the disclosure may comprise, consist of, or consist essentially of SEQ ID NO:32. In certain aspects, the fusion protein may comprise an amino acid sequence at last 80%, at least 90%, at least 95%, at least 97% or at least 99%, identical to SEQ ID NO:35 or 36, wherein the fusion protein may comprise one or more amino acids selected from the group consisting of an alanine at a position corresponding to position 11 of SEQ ID NO:1, an alanine at a position corresponding to position 15 of SEQ ID NO:1, a lysine at a position corresponding to position 22 of SEQ ID NO: 1, a lysine at a position corresponding to position 44 of SEQ ID NO:1, a methionine at a position corresponding to position 52 of SEQ ID NO:1, a glutamine at a position corresponding to position 69 of SEQ ID NO:1, and a lysine at a position corresponding to position 107 of SEQ ID NO:1. In certain aspects, the fusion protein may comprise A122, A126, K133, K155, M163, Q180 and K218. In certain aspects, the fusion protein may comprise A11, A15, K22, K44, M52, Q69, and K107. In certain aspects, the fusion protein may comprise SEQ ID NO:35 or 36.

In certain aspects, the fusion protein may comprise a self-assembling protein of the disclosure, joined to an immunogen of the disclosure and a CSP protein, wherein the fusion protein comprises an amino acid sequence at least 80%, at least 85%, at least 90%, at least 95%, at least 97%, at least 99%, or 100%, identical to SEQ ID NO:32 or SEQ ID NO:33.

One aspect of the disclosure is a nucleic acid molecule encoding a modified 6C domain of the disclosure, an isolated protein of the disclosure, an immunogen of the disclosure, a modified CSP of the disclosure, or a fusion protein of the disclosure. In certain aspects, a modified 6C domain of the disclosure, an isolated protein of the disclosure, a modified CSP of the disclosure, or a fusion protein of the disclosure may be expressed by a nucleic acid construct of the disclosure. As used herein a nucleic acid construct is a recombinant expression vector, i.e., a vector linked comprising a nucleic acid molecule encoding a modified domain, an isolated protein or a fusion protein, such that the nucleic acid molecule can affect expression of the encoded modified domain, isolated protein or fusion protein when the nucleic acid construct is administered to, for example, a subject or an organ, tissue or cell. The vector may also enable transport of the nucleic acid molecule to a cell within an environment, such as, but not limited to, an organism, tissue, or cell culture. A nucleic acid construct of the present disclosure is produced by human intervention. The nucleic acid construct can comprise DNA, RNA or variants thereof. The vector may be a DNA plasmid, a viral vector, or other vector. In one aspect, a vector can be a cytomegalovirus (CMV), retrovirus, adenovirus, adeno-associated virus, herpes virus, vaccinia virus, poliovirus, Sindbis virus, or any other DNA or RNA virus vector. In one aspect, a vector can be a pseudotyped lentiviral or retroviral vector. In one aspect, a vector can be a DNA plasmid. In one aspect, a vector can be a DNA plasmid comprising viral components and plasmid components to enable nucleic acid molecule delivery and expression. Methods for the construction of nucleic acid constructs of the present disclosure are well known. See, for example, Molecular Cloning: a Laboratory Manual, 3rd edition, Sambrook et al. 2001 Cold Spring Harbor Laboratory Press, and Current Protocols in Molecular Biology, Ausubel et al. eds., John Wiley & Sons, 1994. In one aspect, the vector is a DNA plasmid, such as a CMV/R plasmid such as CMV/R or CMV/R 8 KB (also referred to herein as CMV/R 8 kb). Examples of CMV/R and CMV/R 8 kb are provided herein. CMV/R is also described in U.S. Pat. No. 7,094,598 B2, issued Aug. 22, 2006.

As used herein, a nucleic acid molecule may comprise a nucleic acid sequence that encodes a modified 6C domain of the disclosure, an isolated protein of the disclosure, an immunogen of the disclosure, a modified CSP of the disclosure, or a fusion protein of the disclosure. A nucleic acid molecule may be produced recombinantly, synthetically, or by a combination of recombinant and synthetic procedures. A nucleic acid molecule of the disclosure can have a wild-type nucleic acid sequence or a codon-modified nucleic acid sequence to, for example, incorporate codons better recognized by the human translation system. In one embodiment, a nucleic acid molecule can be genetically engineered to introduce, or eliminate, codons encoding different amino acids, such as to eliminate codons that encode an N-linked glycosylation site. Methods to produce nucleic acid molecules of the disclosure are known in the art, particularly once the nucleic acid sequence is known. It is to be appreciated that a nucleic acid construct can comprise one nucleic acid molecule or more than one nucleic acid molecule. It is also to be appreciated that a nucleic acid molecule can encode one protein or more than one protein.

One aspect of the disclosure is a nanoparticle comprising a modified 6C domain of the disclosure, an isolated protein of the disclosure, an immunogen of the disclosure, a modified CSP of the disclosure, or a fusion protein of the disclosure. In certain aspects, the nanoparticle may comprise a SA monomeric subunit disclosed herein joined to a modified 6C domain of the disclosure, an isolated protein of the disclosure, an immunogen of the disclosure, a modified CSP of the disclosure, or a fusion protein of the disclosure. In certain aspects, the nanoparticle is an octahedron. In certain aspects, the nanoparticle elicits an immune response to P48/45 and/or CSP. In certain aspects, the nanoparticle elicits a neutralizing immune response to a Plasmodium spp. In certain aspects, the SA monomeric subunit may comprise at least 50, at least 100 amino acids, or at least 150 contiguous amino acids from an amino acid sequence selected from the group consisting of SEQ ID NOS: 22, 23, 24 and 40, wherein the SA monomeric subunit self-assembles into a nanoparticle. In certain aspects the SA monomeric subunit may comprise an amino acid sequence at least 85%, at least 90%, at least 95%, at least 97% at least 99% identical to an amino acid sequence selected from the group consisting of SEQ ID NOS: 22, 23, 24 and 40, wherein the SA monomeric subunit self-assembles into a nanoparticle. In one embodiment, the SA monomeric subunit may comprise an amino acid sequence selected from the group consisting of SEQ ID NOS: 22, 23, 24 and 40.

In certain aspects, the nanoparticle may comprise a fusion protein comprising a SA monomeric subunit disclosed herein, joined to a modified 6C domain of the disclosure. In certain aspects, the modified 6C domain may be any modified 6C domain disclosed herein. In certain aspects, the modified 6C domain may comprise an amino acid sequence at least 90% identical, or at least 94% identical to SEQ ID NO: 1, wherein the modified 6C domain comprises mutations at one or more amino acid positions selected from the group consisting of an amino acid position corresponding to position 11 of SEQ ID NO:1, an amino acid position corresponding to position 15 of SEQ ID NO:1, an amino acid position corresponding to position 22 of SEQ ID NO:1, an amino acid position corresponding to position 44 of SEQ ID NO:1, an amino acid position corresponding to position 52 of SEQ ID NO:1, an amino acid position corresponding to position 69 of SEQ ID NO:1, and an amino acid position corresponding to position 107 of SEQ ID NO:1. In certain aspects, the modified 6C domain may comprise one or more amino acid positions selected from the group consisting of an alanine at a position corresponding to position 11 of SEQ ID NO:1, an alanine at a position corresponding to position 15 of SEQ ID NO:1, a lysine at a position corresponding to position 22 of SEQ ID NO:1, a lysine at a position corresponding to position 44 of SEQ ID NO:1, a methionine at a position corresponding to position 52 of SEQ ID NO:1, a glutamine at a position corresponding to position 69 of SEQ ID NO:1, and a lysine at a position corresponding to position 107 of SEQ ID NO:1 In certain aspects, the modified 6C domain may comprise one or more amino acid residues selected from the group consisting of A11, A15, K22, K44, M52, Q69, and K107, of SEQ ID NO:5. In certain aspects, the modified 6C domain may comprise A11, A15, K22, K44, M52, Q69, and K107, of SEQ ID NO:5. In certain aspects, the modified 6C domain may comprise one or more amino acid residues selected from the group consisting of an alanine position 11, an alanine at position 15, a lysine at position 22, a lysine at position 44, a methionine at position 52, a glutamine at position 69, and a lysine at position 107. In certain aspects, the modified 6C domain may comprise an alanine position 11, an alanine at position 15, a lysine at position 22, a lysine at position 44, a methionine at position 52, a glutamine at position 69, and a lysine at position 107. In certain aspects, the modified 6C domain may comprise SEQ ID NO:5.

In certain aspects, the nanoparticle may comprise a fusion protein comprising a SA monomeric subunit disclosed herein, joined to a modified CSP domain of the disclosure. In certain aspects, the modified CSP may be any modified CSP disclosed herein. In certain aspects, the modified CSP protein may comprise a CSP junction region, or a portion thereof, joined to a NANP repeat region joined to a portion of the conserved C-terminal region, wherein the NANP repeat region comprises, consists of, or consists essentially of, 3-7 consecutive NANP amino acid sequence repeats, and wherein the modified CSP lacks the N-terminal region of a wild-type (wt) CSP protein. In certain aspects, the junction region, or a portion thereof, may comprise an amino acid sequence corresponding to amino acids 136-147 of SEQ ID NO:27. In certain aspects, the junction region, or a portion thereof, may comprise, consist of, or consist essentially of, amino acid sequence at least 83%, or 100%, identical to SEQ ID NO:37. In certain aspects, the NANP repeat region may comprise five consecutive NANP amino acid sequences. In certain aspects, the portion of the conserved-C terminal region may comprise, consist of, or consist essentially of, an amino acid sequence corresponding to amino acids 313-386 of SEQ ID NO:27. In certain aspects, the junction region, or a portion thereof, may comprise, consist of, or consist of, amino acid sequence at least 90% %, at least 95%, at least 97%, or 100%, identical to SEQ ID NO:38. In certain aspects, the modified CSP may lack an amino acid sequence corresponding to amino acids 1-135, or 18-135, of SEQ ID NO:27. In certain aspects, a modified CSP of the disclosure may comprise, consist of, or consist of, a junction region, or a portion thereof, joined a NANP repeat region, which is joined to a conserved C-terminal region, wherein the junction region, or a portion thereof, comprises, consists of, or consist essentially of, an amino acid sequence at least 83%, or 100%, identical to SEQ ID NO:37, wherein the NANP repeat region consists, or consists essentially of, 3-7 consecutive NANP amino acid sequences, and wherein the conserved-C-terminal region may comprise, consist of, or consist essentially of, an amino acid sequence at least 90% %, at least 95%, at least 97%, or 100%, identical to SEQ ID NO:38. In certain aspects, the modified CSP may lack an amino acid sequence corresponding to amino acids 1-135, or 18-135, of SEQ ID NO: 27.

In certain aspects, the modified CSP of the disclosure may comprise, consist of, or consist essentially of, an amino acid sequence at least at least 90% %, at least 95%, at least 97%, or at least 99% identical to SEQ ID NO:32, wherein the modified CSP comprises a first amino acid sequence at least 83%, or 100% identical to SEQ ID NO:37 joined to a second amino acid sequence consisting of, or consisting essentially of, three, four, five, six or seven consecutive NANP sequences, wherein the second amino acid sequence is joined to a third amino acid sequence comprising, consisting of, or consisting essentially of, an amino acid sequence at least 90% %, at least 95%, at least 97%, or 100%, identical to SEQ ID NO:38. In certain aspects, a modified CSP of the disclosure may comprise, consist of, or consist essentially of SEQ ID NO:32.

In certain aspects, the nanoparticle may comprise a fusion protein comprising, consisting of, or consisting essentially of, an amino acid sequence at last 80%, at least 90%, at least 95%, at least 97% or at least 99%, identical to SEQ ID NO: 35 or 36, wherein the fusion protein may comprise one or more amino acids selected from the group consisting of an alanine at a position corresponding to position 11 of SEQ ID NO: 1, an alanine at a position corresponding to position 15 of SEQ ID NO:1, a lysine at a position corresponding to position 22 of SEQ ID NO:1, a lysine at a position corresponding to position 44 of SEQ ID NO:1, a methionine at a position corresponding to position 52 of SEQ ID NO:1, a glutamine at a position corresponding to position 69 of SEQ ID NO:1, and a lysine at a position corresponding to position 107 of SEQ ID NO:1. In certain aspects, the fusion protein may comprise A122, A126, K133, K155, M163, Q180 and K218. In certain aspects, the fusion protein may comprise A11, A15, K22, K44, M52, Q69, and K107. In certain aspects, the fusion protein may comprise, consist of, or consist essentially of, SEQ ID NO:35 or 36.

One aspect of the disclosure is a composition comprising a modified 6C domain of the disclosure, an immunogen of the disclosure, an isolated protein, a fusion protein, a nucleic acid molecule or a nanoparticle of the disclosure. In certain aspects, compositions of the disclosure may comprise additional components to improve the stability and stability of the composition. Such components include, but are not limited to, diluents, buffers, salts, sugars, detergents, and stabilizers.

Because a modified 6C domain of the disclosure, an immunogen of the disclosure, a modified CSP of the disclosure, an isolated protein, a fusion protein, or a nanoparticles of the disclosure can elicit a neutralizing immune response against Plasmodium spp, they can be used as vaccines to protect individuals against infection by a Plasmodium or to treat individuals infected with a Plasmodium. According to the disclosure a vaccine may comprise a modified 6C domain of the disclosure, an immunogen of the disclosure, an isolated protein, a fusion protein, or a nanoparticle of the disclosure. Thus, one aspect of the disclosure is a vaccine comprising a modified 6C domain of the disclosure, an immunogen of the disclosure, an isolated protein, a fusion protein, a nanoparticle of the disclosure, a nucleic acid molecule of the disclosure, or a composition of the disclosure. Vaccines of the disclosure may also contain other components such as adjuvants, buffers and the like. Although any adjuvant may be used, preferred aspects can contain: chemical adjuvants such as aluminum phosphate, benzyalkonium chloride, ubenimex, and QS21; genetic adjuvants such as the IL-2 gene or fragments thereof, the granulocyte macrophage colony-stimulating factor (GM-CSF) gene or fragments thereof, the IL-18 gene or fragments thereof, the chemokine (C—C motif) ligand 21 (CCL21) gene or fragments thereof, the IL-6 gene or fragments thereof, CpG, LPS, TLR agonists, and other immune stimulatory genes; protein adjuvants such IL-2 or fragments thereof, the granulocyte macrophage colony-stimulating factor (GM-CSF) or fragments thereof, IL-18 or fragments thereof, the chemokine (C—C motif) ligand 21 (CCL21) or fragments thereof, IL-6 or fragments thereof, CpG, LPS, TLR agonists and other immune stimulatory cytokines or fragments thereof; lipid adjuvants such as cationic liposomes, N3 (cationic lipid), monophosphoryl lipid A (MPL1); other adjuvants including cholera toxin, enterotoxin, Fms-like tyrosine kinase-3 ligand (Flt-3L), bupivacaine, marcaine, and levamisole.

One aspect of the disclosure is a method of preventing infection of an individual by a Plasmodium spp, the method comprising administering to the individual a therapeutically effective amount of a modified 6C domain of the disclosure, an isolated protein of the disclosure, an immunogen of the disclosure, a modified CSP of the disclosure, a fusion protein of the disclosure, a composition of the disclosure, a nanoparticle of the disclosure, a nuclei acid molecule of the disclosure, or a vaccine of the disclosure.

One aspect of the disclosure is a method treating an individual against malaria, the method comprising administering to the individual a therapeutically effective amount of a modified 6C domain of the disclosure, an isolated protein of the disclosure, an immunogen of the disclosure, a modified CSP of the disclosure, a fusion protein of the disclosure, a composition of the disclosure, a nanoparticle of the disclosure, a nuclei acid molecule of the disclosure, or a vaccine of the disclosure.

One aspect of the disclosure is a method treating an individual infected with a Plasmodium spp, the method comprising administering to the individual a therapeutically effective amount of a modified 6C domain of the disclosure, an isolated protein of the disclosure, an immunogen of the disclosure, a modified CSP of the disclosure, a fusion protein of the disclosure, a composition of the disclosure, a nanoparticle of the disclosure, a nuclei acid molecule of the disclosure, or a vaccine of the disclosure.

One aspect of the disclosure is a method treating an individual for malaria, the method comprising administering to the individual a therapeutically effective amount of a modified 6C domain of the disclosure, an isolated protein of the disclosure, an immunogen of the disclosure, a modified CSP of the disclosure, a fusion protein of the disclosure, a composition of the disclosure, a nanoparticle of the disclosure, a nuclei acid molecule of the disclosure, or a vaccine of the disclosure.

One aspect of the disclosure is use of a modified 6C domain of the disclosure, an isolated protein of the disclosure, an immunogen of the disclosure, a modified CSP of the disclosure, a fusion protein of the disclosure, a composition of the disclosure, a nanoparticle of the disclosure, a nuclei acid molecule of the disclosure, or a vaccine of the disclosure in preventing infection of an individual with Plasmodium.

One aspect of the disclosure is use of a modified 6C domain of the disclosure, an isolated protein of the disclosure, an immunogen of the disclosure, a modified CSP of the disclosure, a fusion protein of the disclosure, a composition of the disclosure, a nanoparticle of the disclosure, a nuclei acid molecule of the disclosure, or a vaccine of the disclosure for treating or preventing malaria.

One aspect of the disclosure is a kit for practicing methods of the disclosure. A kit may include, for example, a modified 6C domain of the disclosure, an isolated protein of the disclosure, an immunogen of the disclosure, a modified CSP of the disclosure, a fusion protein of the disclosure, a nucleic acid molecule of the disclosure, an expression vector of the disclosure, a cell of the disclosure, a nanoparticle of the disclosure, a composition of the disclosure, or a vaccine composition of the disclosure, as well components for making such components. As such, kits can include, for example, primers, nucleic acid molecules, expression vectors, DNA constructs encoding proteins of the present disclosure, cells, buffers, reagents, syringes, and directions for using any of said components. It should be appreciated that a kit may comprise more than one container comprising any of the aforementioned, or related, components. For example, certain parts of the kit may require refrigeration, whereas other parts may be stored at room temperature. Thus, as used herein, a kit may comprise components sold in separate containers by one or more entity, with the intention that the components contained therein be used together.

Publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

This written description uses examples to disclose the invention, including the best mode, and to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

### EXAMPLES

### Methods

### Example 1. Prediction of the D3 Interaction Surface with D2

A model of the Pfs48/45 D2/D3 interaction surface was created by aligning the structures of Pfs48/45-D3 and the C-terminal 6-cys domains of P12 and P41. P12 and P41 are tandem 6-Cys domains with similar D2/D3 interaction interfaces, suggesting this interface is relatively conserved in tandem 6-Cys domains. Residues of Pfs48/45-D3 that contact either N-terminal 6-Cys domain of P12 or P41 were then defined as a putative interface for design. Contact residues, and other interfaces, were defined as those that have a >1 Å change in solvent accessible surface area upon removal of the interacting partner, and calculations were performed in PyMOL.

### SPEEDesign Computational Steps

The SPEEDesign pipeline categorizes each residue as fixed, intermediate, or deep search to define the amino acids identities sampled at each position during the computational design process. Pfs48/45-D3 residues that form the interface with the neutralizing mAb 85RF45.1 were defined as fixed, leaving them unchanged through the design process.

Residues exposed upon the extraction of a domain from a larger protein require are defined as deep search in SPEEDesign. Since these residues are not exposed in homologous proteins, conservation or evolutionary-based design principles may not prove sufficient to redesign these new non-natural surfaces. For Pfs48/45-D3, these newly exposed residues were defined by modeling the D2/D3 interface (see above). The NxS/T motif and all residues that contact the glycan were also defined as deep search, allowing them to sample all amino acids, except cysteine.

All other residues were defined as intermediate. These residues were allowed to vary to a limited extent that is driven by conservation and evolutionary analysis of similar protein sequences to identify potential amino acid changes. These amino acid constraints were used to create a ResFile for each strategy that was used by the FastDesign module in ROSETTA.

SPEEDesign uses the four ROSETTA design strategies previously described (Dickey, T. H. et al. Design of the SARS-COV-2 RBD vaccine antigen improves neutralizing antibody response. Sci Adv 8, eabq8276, doi: 10.1126/sciadv.abq8276 (2022). All ROSETTA strategies leave fixed residues unchanged to preserve neutralizing epitopes, and each strategy differs in the amino acid changes allowed for the intermediate and deep search categories of residues

Decoy clustering was performed as described previously (Ibid. For each computational strategy, decoys with scores in the 95th percentile were clustered by sequence similarity using CD-HIT and the top scoring decoy form each cluster was selected as a representative sequence (Li, W. & Godzik, A. Cd-hit: a fast program for clustering and comparing large sets of protein or nucleotide sequences. Bioinformatics 22, 1658-1659, (2006)). The number of clusters was selected based on the sequence diversity produced in each computational strategy.

### Example 2. SPEEDesign In Vitro Screening

Immunogen screening was performed as described previously. Synthetic DNA coding for secreted immunogens was cloned (GenScript) into a customized pHL-sec expression plasmid. pHL-sec was a gift from Edith Yvonne Jones (Addgene plasmid #99845; http://n2t.net/addgene: 99845; RRID: Addgene_99845). Plasmid was transfected into human expi293F cells and grown in a 96-well plate according to manufacturer instructions (ThermoFisher Scientific). Cell-free supernatant was harvested after 4 days of expression.

Cell-free supernatant was diluted 1:3 in phosphate buffered saline (137 mM NaCl, 2.7 mM KCl, 10 mM Na₂HPO₄, 1.8 mM KH₂PO₄) with 0.05% Tween20 (PBST)+2% bovine serum albumin (BSA) and added to nickel-nitrilotriacetic acid (Ni-NTA) HisSorb Plates (Qiagen) to capture His-tagged immunogens. After incubation for 1 hour at room temperature, plates were washed three times with PBST. Neutralizing epitopes were probed using a TB31F-Fc (IgG1) protein (0.05 μg/well). After incubation for 1 hour at room temperature, plates were washed three times with PBST and 200 μl 1:5000 peroxidase-conjugated anti-human IgG was added (Jackson ImmunoResearch Laboratories, Inc. Cat. #109-035-098). Plates were incubated 30 minutes at room temperature and washed three times with PBST. Finally, 70 μl Tetramethylbenzidine (TMB) (MilliporeSigma) was added and incubated 2 minutes at room temperature before quenching with 70 μl 0.16 M H2SO4. Absorbance at 450 nm was measured using a Biotek Synergy H1 plate reader.

### Example 3. Amino Acid Reversion to Native Sequence

Amino acids that were changed in the original immunogens 15 and 17 were individually reverted to their native identity. The energetic effect of this reversion was calculated using ROSETTA filterscan mover. The suffix of the reversion construct indicates the energetic penalty that was allowed during the reversion process. For example, 15-2 contains all individual reversion mutations with a delta filter threshold of 2 in filterscan, and 17-4 contains all mutations with a delta filter threshold of 4 in filterscan.

### Example 4. Immunogen Expression, Purification, and Calculation of Yields

Recombinant immunogens were expressed in expi293F cells, as described for SPEEDesign in vitro screening above. Cell-free supernatant was harvested 4 days after transfection and His-tagged antigens were purified by gravity chromatography using Ni Sepharose excel resin according to manufacturer instructions (Cytiva). Antigens were further purified by size-exclusion chromatography using a Superdex 75 Increase 10/300 GL column equilibrated in 1×PBS. Fractions corresponding to monomeric immunogen were pooled, snap frozen in liquid nitrogen, and stored at −80° C.

Transfection, expression, and purification of original designs was performed in triplicate on three separate days to calculate immunogen purification yields. Each replicate consisted of a 30 mL culture, and yields were calculated by integrating the area under the monomeric peak on the Abs280 chromatogram during size-exclusion chromatography. These yields closely matched yields calculated from pooled fractions. Purification yields for reversion mutants was calculated after nickel purification by measuring the Abs280 of eluant. Extinction coefficients were calculated using the ExPASy ProtParam tool.

### TB31F Cloning, Expression, and Purification

TB31F was created by fusing the variable regions to the human IGHG*01 or IGLC2*02 constant regions and cloning into the pHL-sec plasmid (GenScript). Heavy and light chain plasmids were mixed in equal amounts and transfected into expi293F cells according to manufacturer instructions and cell-free supernatant was harvested after 4 days of expression.

Cell-free supernatant was batch incubated with protein A agarose resin (GoldBio) for 1 hour at room temperature. Resin was collected and washed with 10 column volumes (CV) protein A IgG binding buffer (ThermoFisher Scientific). Protein was eluted with 10 CV IgG elution buffer (ThermoFisher Scientific) and neutralized with 1 CV 1 M Tris pH 9.0. Antibody was concentrated and buffer exchanged into PBS using an Amicon centrifugal filter (MilliporeSigma).

### Nanoparticle Expression and Purification

Nanoparticles were created by genetically fusing ferritin to the C-terminus of Pfs48/45 immunogens. There is a GSGGGG (SEQ ID NO:25) linker between the immunogens and ferritin. The ferritin construct is a previously described engineered construct from bullfrog and H. pylori ferritin. All constructs were created without additional purification or solubility tags.

Proteins were expressed in expi293F cells, as described above. Cell-free supernatant was harvested after 4 days of expression at 37° C. and concentrated using 100,000 kDa molecular weight cutoff Amicon centrifugal filter units. Concentrated supernatant was purified by size-exclusion chromatography using a Superose 6 Increase 10/300 GL column (Cytiva) equilibrated in 1×PBS. Fractions corresponding to assembled nanoparticle were pooled, snap frozen in liquid nitrogen, and stored at −80° C.

### Differential Scanning Fluorimetry

DSF was performed using the Protein Thermal Shift Dye Kit according to manufacturer instructions (ThermoFisher Scientific). Final reactions contained 0.4 mg/mL purified immunogen, 1× Protein Thermal Shift buffer, 1× Thermal Shift Dye, and 0.63× PBS. Fluorescence was monitored using a 7500 Fast Real-Time PCR system (ThermoFisher Scientific) as the temperature was increased from 25° C. to 95° C. at a ramp rate of 1%. Melting temperature (Tₘ) was calculated using the Boltzmann method. DSF reactions were performed in technical quadruplicate on each plate and in biological triplicate using three different protein preps on 3 separate days for original designs. Reversion mutants were analyzed as a single biological replicate. Technical replicates were averaged to calculate the Tₘ for a biological replicate, and biological replicates were averaged to calculate the reported Tₘ.

### Biolayer Interferometry

The binding affinity of purified immunogens to TB31F was measured using a kinetic BLI assay using an Octet-Red96e (Sartorius), as described previously. TB31F was buffer exchanged into HBS-EP buffer (10 mM Na-HEPES pH 7.4, 150 mM NaCl, 3 mM EDTA, 0.005% v/v P20 surfactant) using Zeba spin desalting columns (ThermoFisher Scientific). TB31F was loaded onto Anti-hIgG Fc Capture (AHC) biosensors (Sartorius) over the course of 300 seconds, until reaching a signal of ~0.6 nm. BLI pins were then immersed in immunogens 2-fold serially diluted in HBS-EP buffer (30 nM to 0.469 nM). After 300 seconds, pins were immersed in HBS-EP buffer to measure dissociation. Association rate (kₐ), dissociation rate (k_{dis}), and dissociation constant (K_{D}) were globally fit using a 1:1 binding model in Data Analysis HT 12.0 (Sartorius). Three independent protein preps (biological replicates) were each measured in technical triplicate. Values reported are the average and standard deviation between biological replicates.

### Negative-Stain Electron Microscopy

Purified nanoparticles (0.01 mg/ml) were adsorbed on a glow discharged 300 mesh carbon-coated copper grids (EMD Science) for 30s followed by NanoW (Nanoprobes) staining. Raw Micrographs were recorded in Thermo Scientific Tecnai T20 microscope equipped with a charge-coupled device (CCD) camera. Particles were auto-picked using Gautomatch (http://www.mrc-lmb.cam.ac.uk/kzhang/) and 2D classes were generated using RELION 3.0.

### Rat Immunizations

For CFA/IFA studies, immunizations were performed by Noble Life Sciences in an AAALAC-accredited facility under the guidelines and approval of the Institutional Animal Care and Use Committee (IACUC) approved, and under OLAW assured conditions. Male Wistar rats (Envigo) weighing 225-250 grams were immunized by subcutaneous injection, delivering 40 μg antigen per animal in a 100 μl volume. Monomeric antigens were purified, as described above, snap frozen in liquid nitrogen, and stored at −80. On the day of immunization, antigen was thawed and formulated as a 1:1 ratio in Complete Freund's Adjuvant (MilliporeSigma) on day 0 and Incomplete Freund's Adjuvant (MilliporeSigma) on days 21 and 42. Blood was collected on indicated days, and serum was separated and stored at −80° C.

AddaSO3 immunizations were performed in an AAALAC-accredited facility under the guidelines and approval of the Institutional Animal Care and Use Committee (IACUC) at the National Institutes of Health. Nine-week-old female CD (Charles River Laboratories) per group were immunized with 1 μg antigen in a 100 μl volume by subcutaneous injections given on days 0 and 21. Nanoparticle antigens were purified, as described above, snap frozen in liquid nitrogen, and stored at −80. On the day of immunization, antigen was thawed and formulated as a 1:1 ratio in AddaS03, according to manufacturer recommendations (Invivogen). Blood was collected on indicated days, and serum was separated and stored at −80° C.

### Serum Antibody Titer ELISAs

Nunc MaxiSorp plates (ThermoFisher Scientific) were coated with 100 μl 0.005 mg/mL purified WT Pfs48/45-D3 diluted in 50 mM Na-carbonate pH 9.5. Plates were incubated overnight at 4° C. then washed three times with PBST. Plates were blocked 1 hour at room temperature with 2% BSA in PBST then washed three times with PBST. Serum was diluted in 2% BSA in PBST and 100 μl was added to each well. After 1 hour incubation at room temperature, plates were washed three times with PBST and 200 μl 1:10,000 peroxidase-conjugated anti-rat IgG (Jackson ImmunoResearch Laboratories, Inc.) was added. Plates were incubated 30 minutes at room temperature and washed three times with PBST. Finally, 70 μl Tetramethylbenzidine (TMB) (MilliporeSigma) was added and incubated 20 minutes at room temperature before quenching with 70 μl 0.16 M H2SO4. Absorbance at 450 nm was measured using a Biotek Synergy H1 plate reader.

Pooled serum from rats immunized with WT D3 in CFA/IFA was used as a standard curve on each plate to calculate the antibody titers of individual animals in all groups. One antibody unit (AU) was defined as the dilution of the standard serum required to achieve an Abs450 value of 1. Each plate included triplicate 2-fold serial dilutions of the standard serum from 20 to 0.01 AU. Serum from each animal was diluted such that the Abs450 fell in the informative portion of the standard curve between 0.1 and 2.0. The Abs450 values for the standard curve were fit to a 4-parameter logistic curve, which was used to convert Abs450 values to AU for each individual animal. AU values for each individual animal were measured in triplicate on separate plates and the average was reported.

### Standard Membrane Feeding Assay (SMFA)

Functional activity of immune sera was assayed by an ex vivo standard membrane feeding assay (SMFA) in terms of their transmission reducing activity (TRA, reduction in mosquito infection intensity) as described previously. In vitro 14-16-day-old gametocyte culture of P. falciparum (NF54 line) was diluted with washed O+ red blood cells (RBCs) from a malaria naïve donor and AB+ human naïve serum pool to achieve 0.12±0.05% concentration of Stage V gametocytes and 50% hematocrit. Test samples, containing 30 μL of test serum and 30 μL of naïve human AB+ serum pool (60 μL total volume each), were prepared in advance. Each test sample (60 μL) was mixed with 200 μL aliquot of diluted culture and immediately fed to pre-starved (~24 h) 3-8-day-old Anopheles stephensi (Nijmegen strain) mosquitoes through a membrane feeding device connected to a 40° C. circulating water bath. After feeding, mosquitoes were maintained at 26° C. and 80% humidity conditions to allow for the development of parasites. On day 8 mosquito midguts were dissected and stained with 0.05% mercurochrome and the number of oocysts on each midgut were counted. TRA was calculated by the following formula:

$$T R A = 100 \times ( \frac{Mean Oocyst {Number}_{neg ctrl} - Mean Oocyst {Number}_{test}}{Mean Oocyst {Number}_{neg ctrl}}$$

where the negative control (neg ctrl) feed used serum from rats immunized with adjuvant only.

SMFAs were performed with heat-inactivated and not heat-inactivated AB+naïve human serum pool and rat test sera were heat-inactivated. RBCs from a malaria naïve donor and AB+ human naïve sera for the pool were received from Interstate Blood Bank, Memphis, Tennessee.

### Results

Non-Glycosylated Immunogens have Improved Yield and Thermostability, and Retain the Neutralizing Epitope

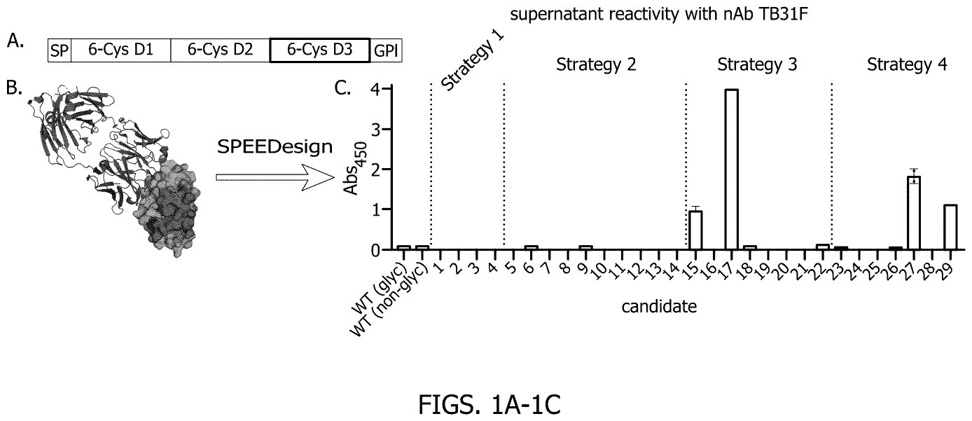

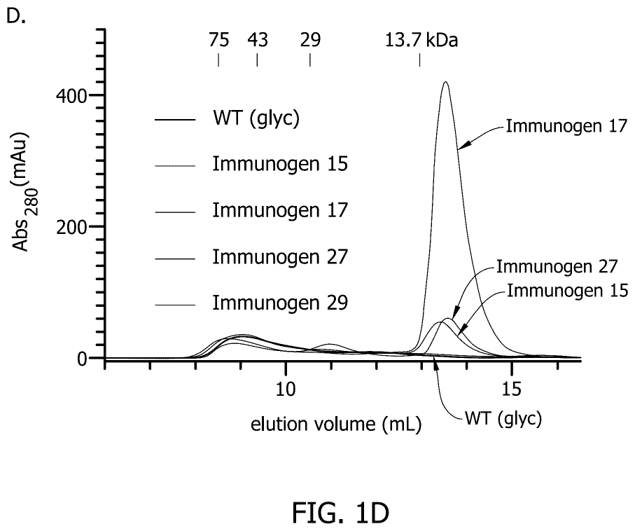

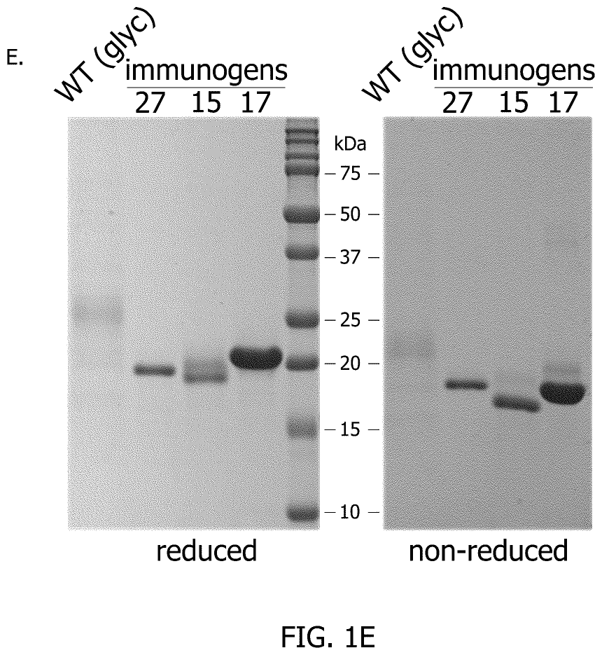

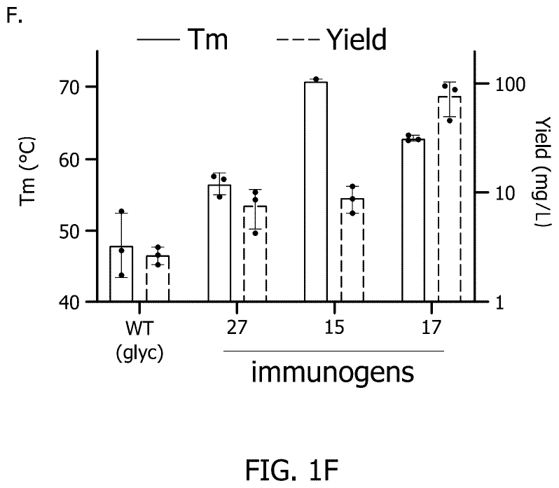

SPEEDesign was used to create an enhanced pfs48/45 D3 immunogen (FIG. 1B). The goal was to create a pfs48/45 D3 immunogen that could be expressed as a non-glycosylated stand-alone antigen in eukaryotic systems without any fusion partners that may detract from the immune response to pfs48/45. The standard SPEEDesign protocol was modified to include ablation of the glycosylation site during design.

SPEEDesign first requires a definition of the role of each amino acid within the protein, and consequently its mutability during the computational design process. The neutralizing epitope of mAb 85RF45.1 was retained by disallowing mutation during the computational design process. The residues predicted to contact the N-terminal domain 2 of pfs48/45, including the location of the stabilizing NAG303, were thoroughly searched during the design process to identify changes that would stabilize domain 3 upon extraction from the full-length protein. All remaining residues were allowed to sample a sequence space limited by energetic and evolutionary constraints.

SPEEDesign uses four different computational strategies that differ in their depth of design for each class of residue to generate 40,000 computational decoys. A clustering algorithm was then used to identify 29 candidates that sample a diverse sequence space (FIG. 1C). These candidates were screened in a mammalian expression system by capturing His-tagged immunogens from cell-free supernatant using a Ni-coated plate and measuring binding of the neutralizing mAb TB31F by enzyme-linked immunoassay (ELISA). Screening identified four candidates that had reactivity to TB31F greater than WT D3. All four candidates derive from strategies 3 and 4, which sample an intermediate sequence space that is less restrictive than strategy 1 and more confined than strategy 2 (see methods).

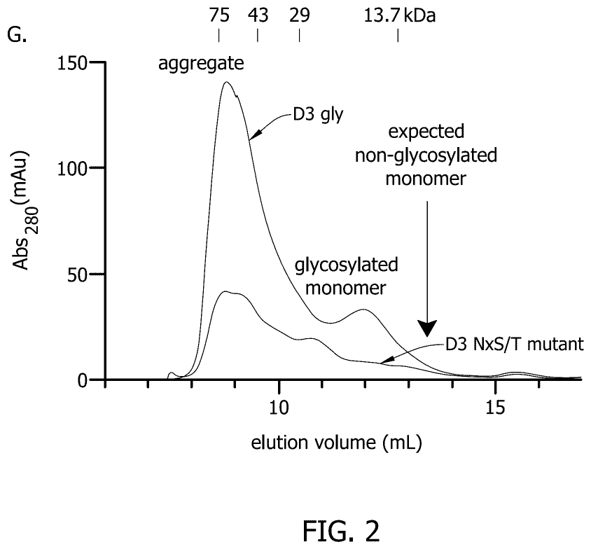

Three lead immunogens (15, 17, and 27) could be expressed and purified as monomeric proteins with yields far greater than WT D3 (FIGS. 1D-1F). Mutation of the N-linked glycosylation sites in WT D3 resulted in no monomeric protein, and the glycosylated form had low yields of 2-3 mg/L, consistent with previous observations (FIG. 1F and FIG. 2). Conversely, non-glycosylated immunogens 15 and 27 had yields of 9 and 8 mg/L, respectively, and immunogen 17 had an average yield of 77 mg/L, more than 25-fold higher than WT D3.

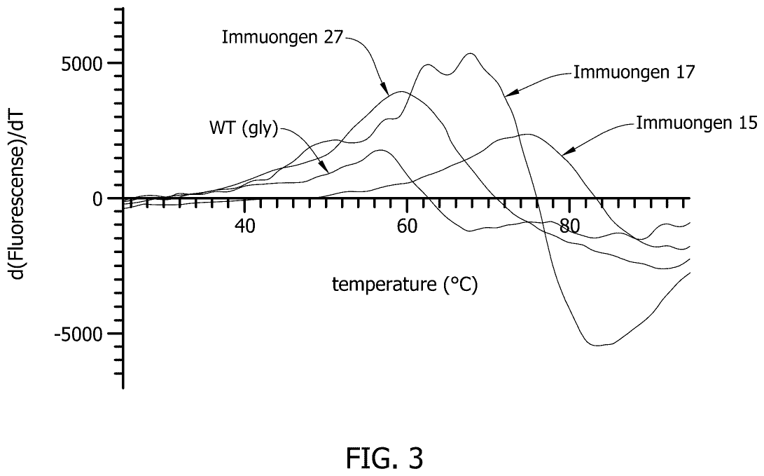

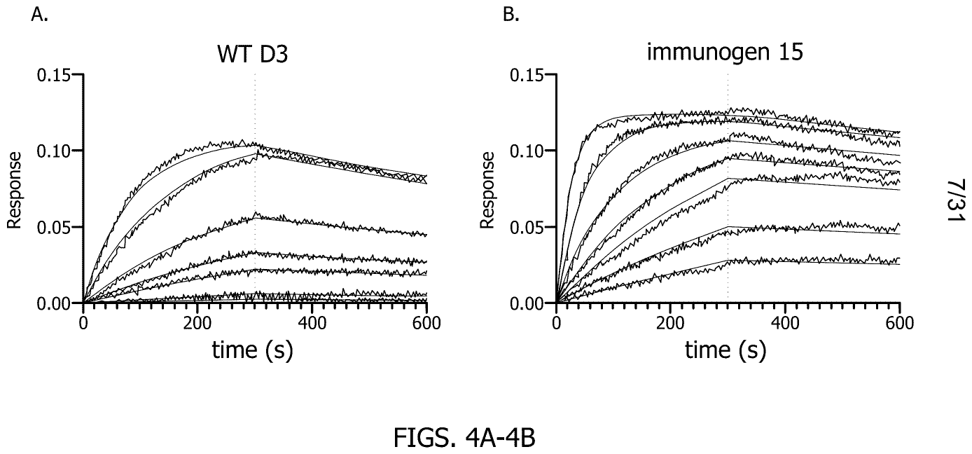

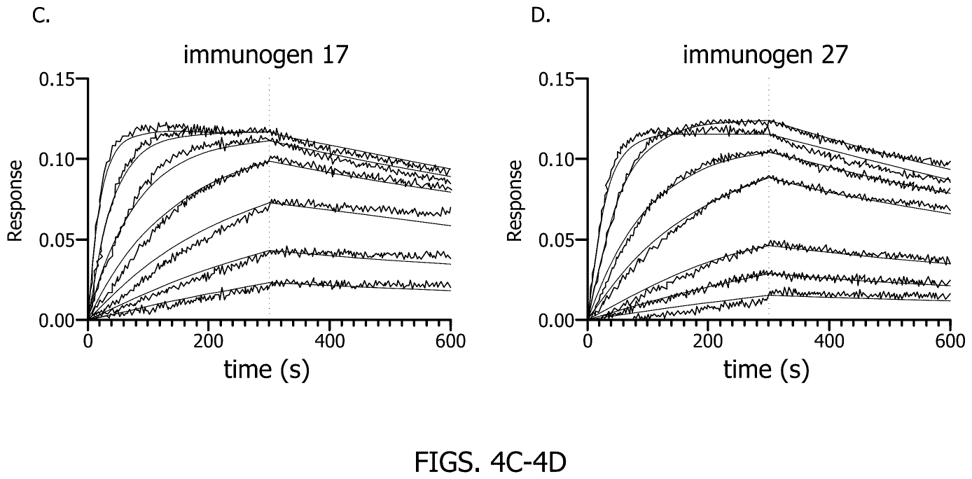

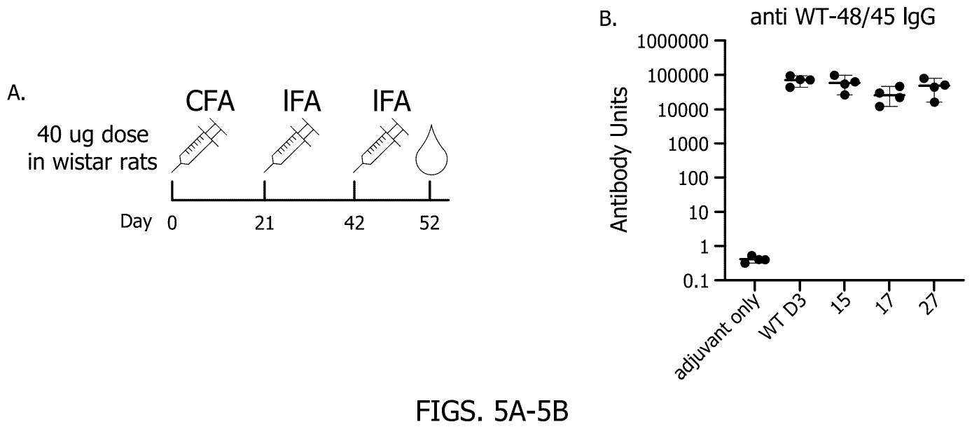

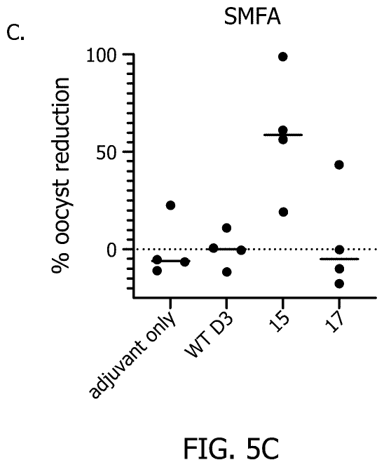

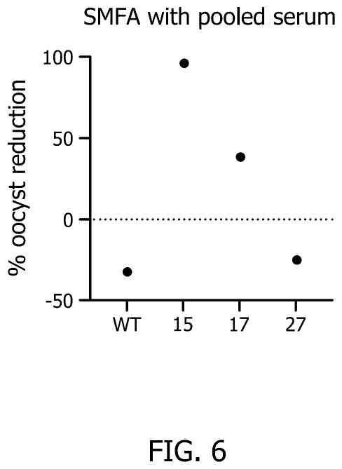

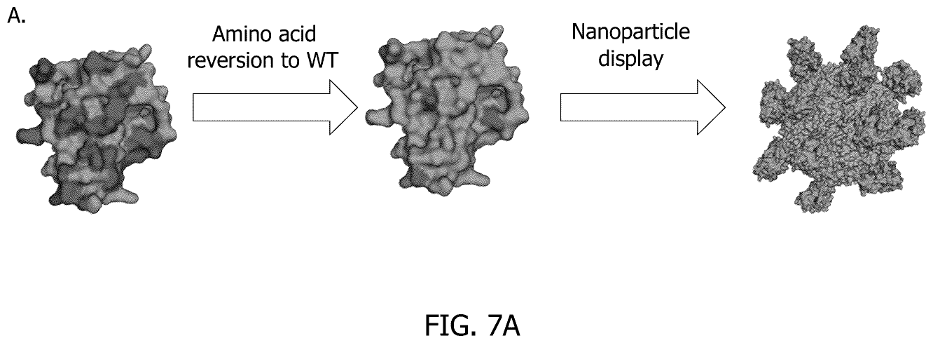

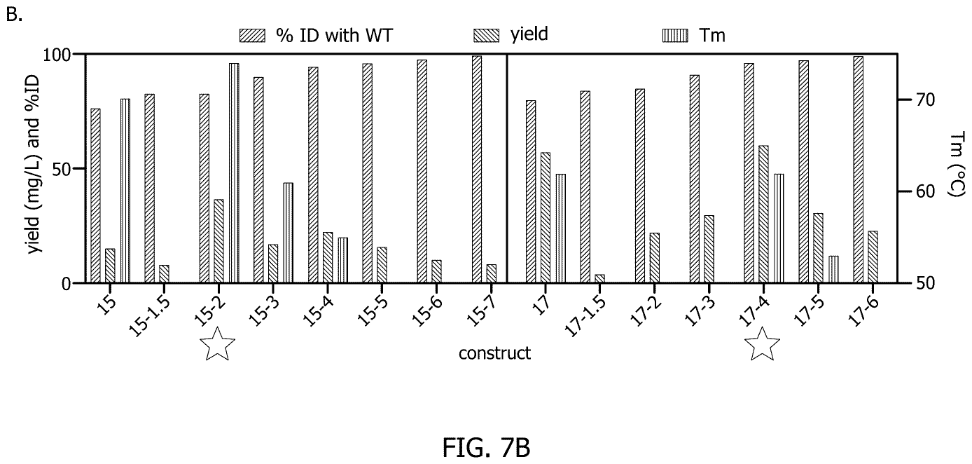

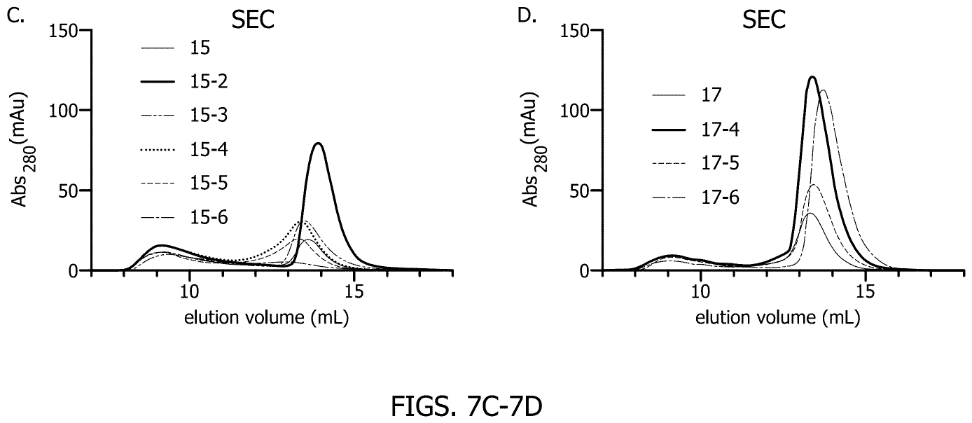

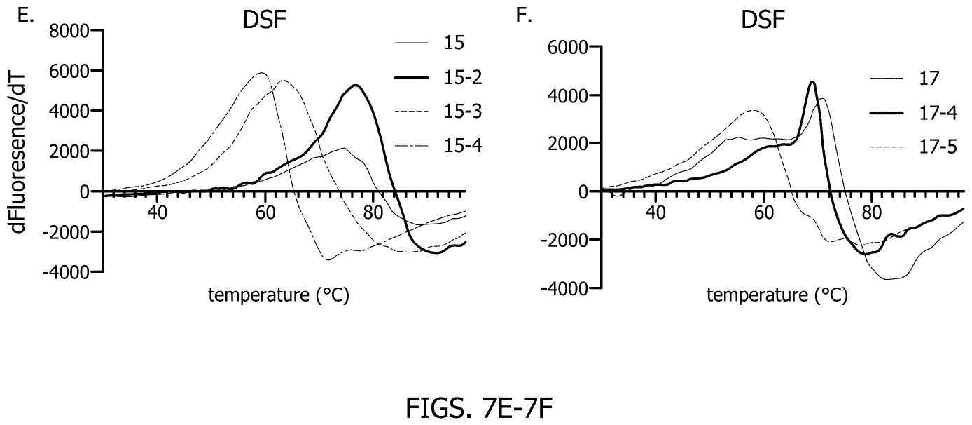

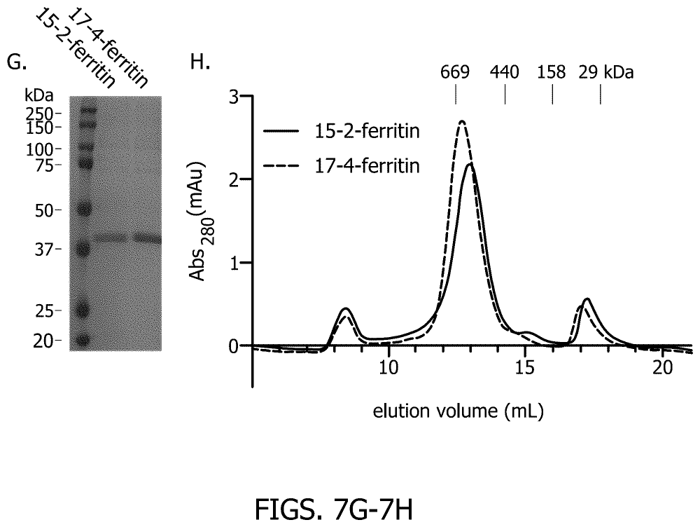

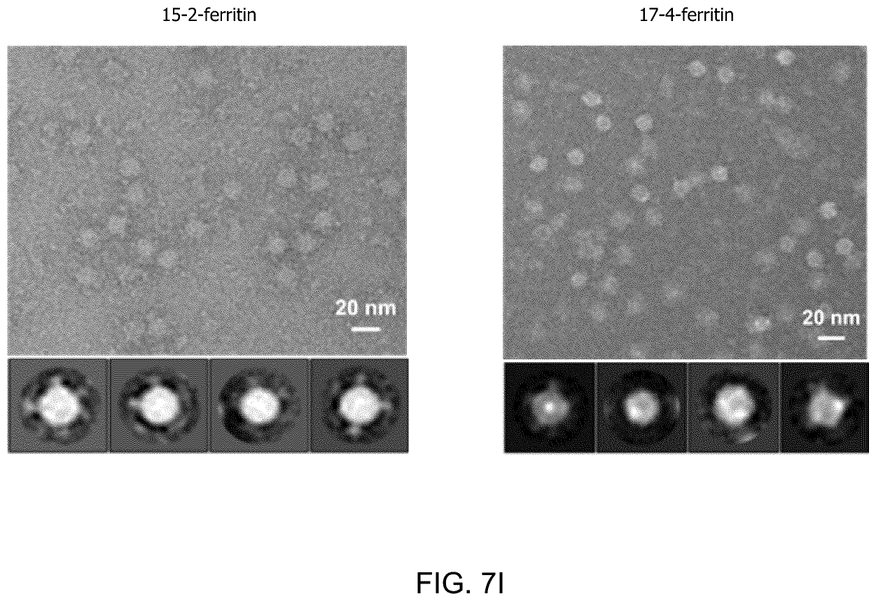

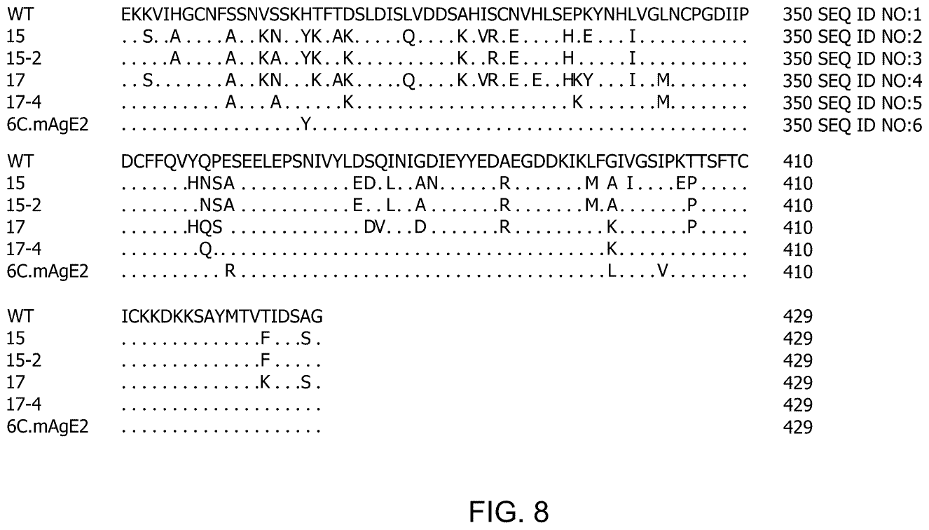

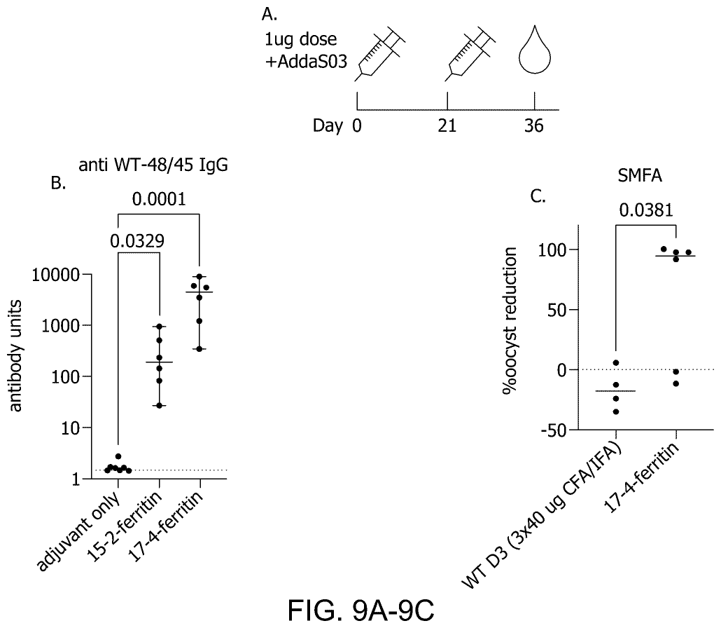

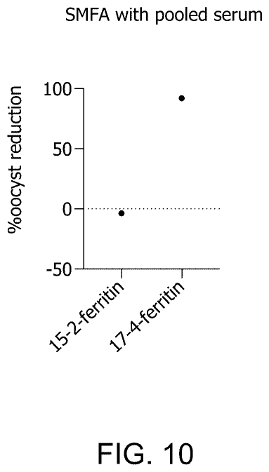

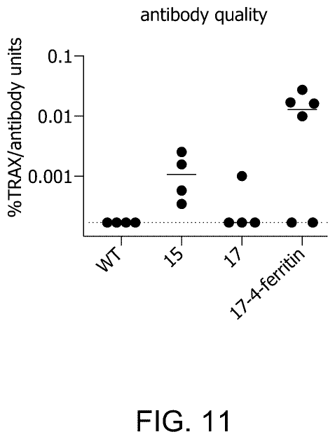

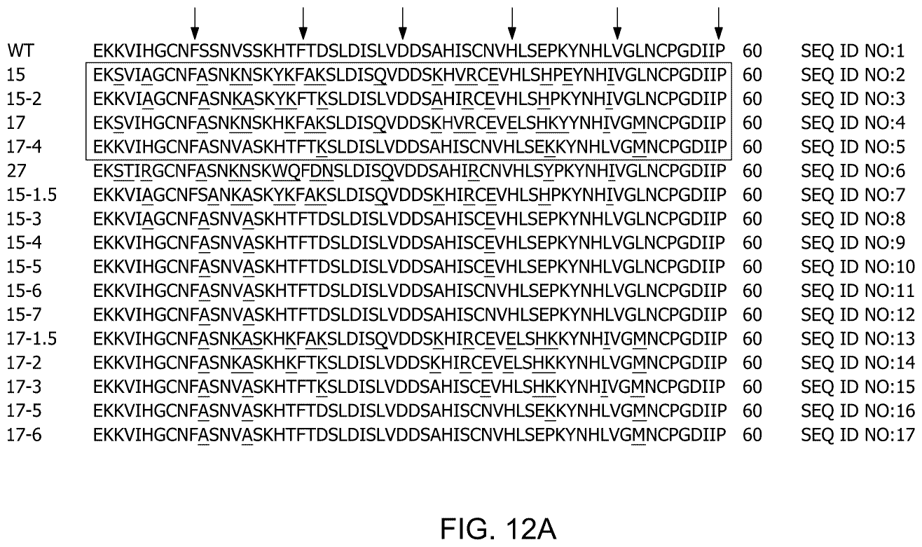

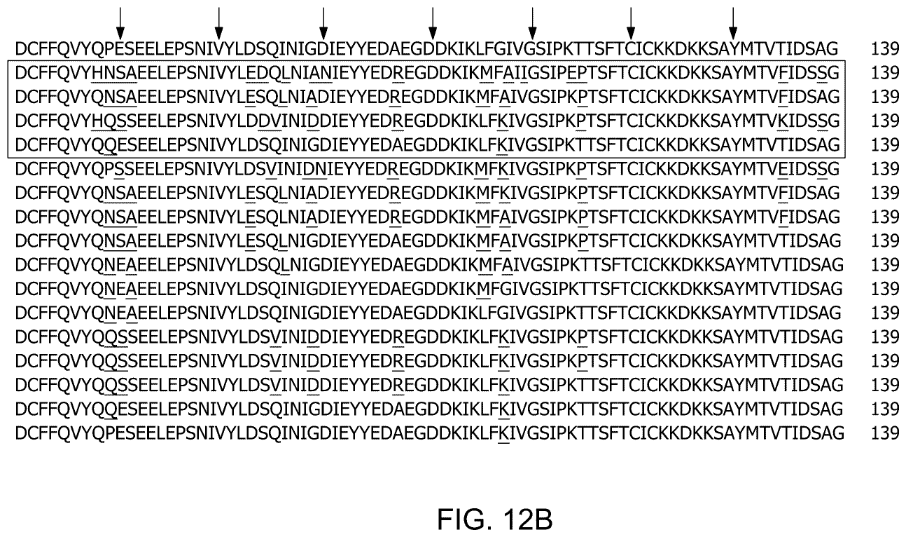

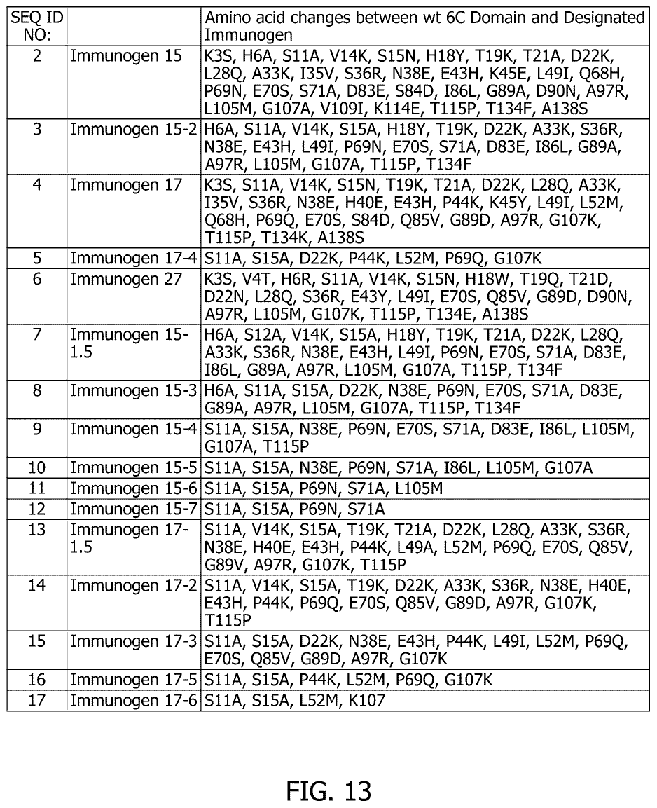

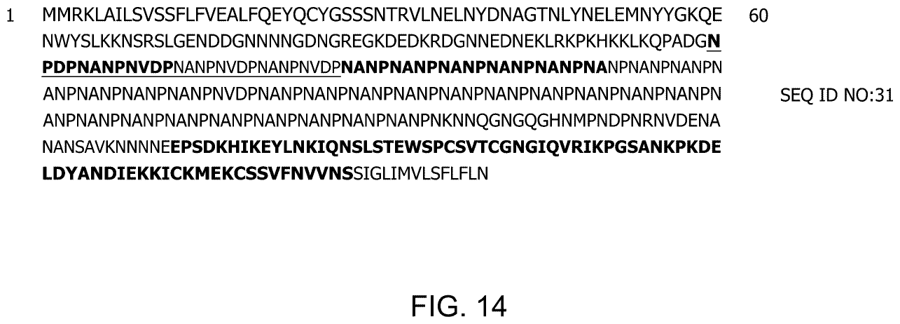

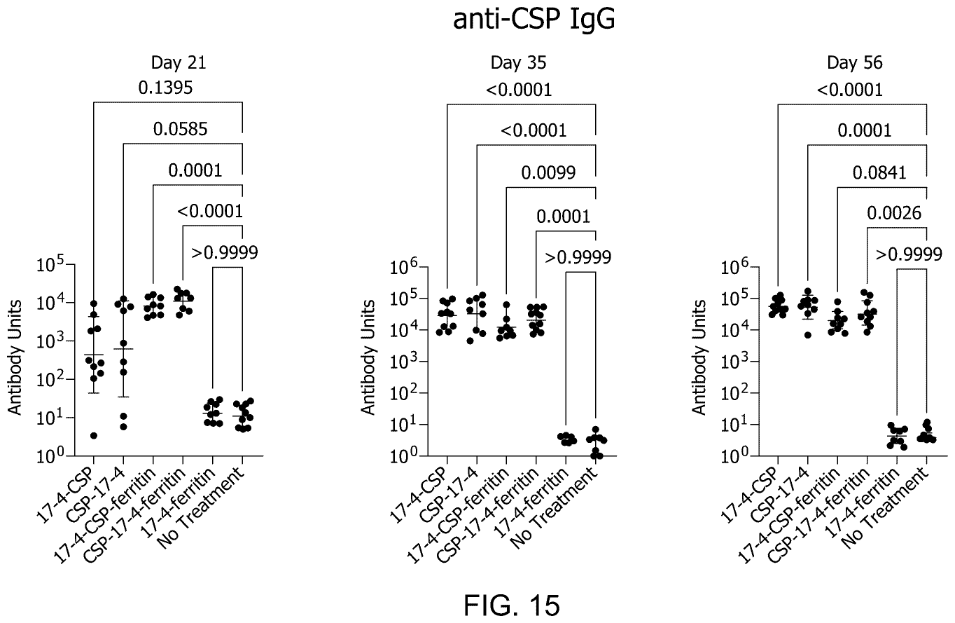

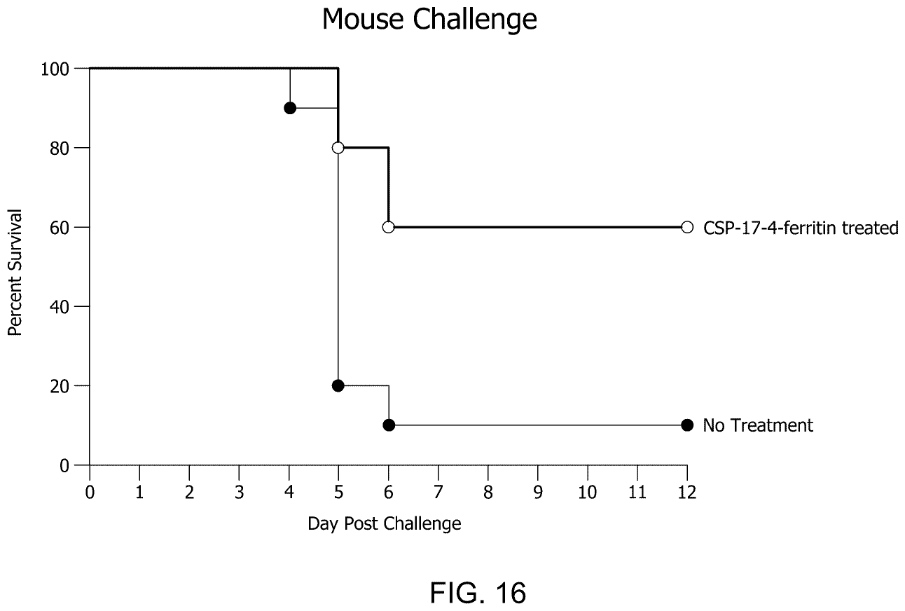

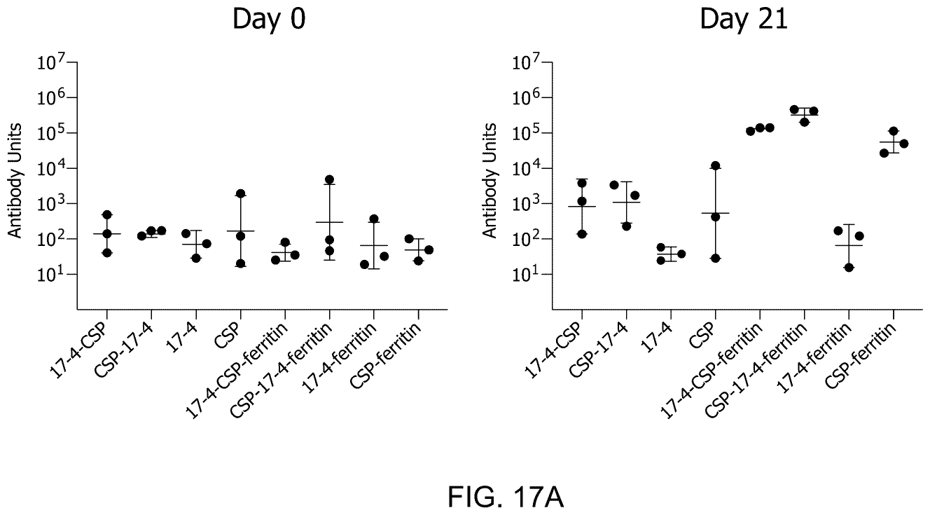

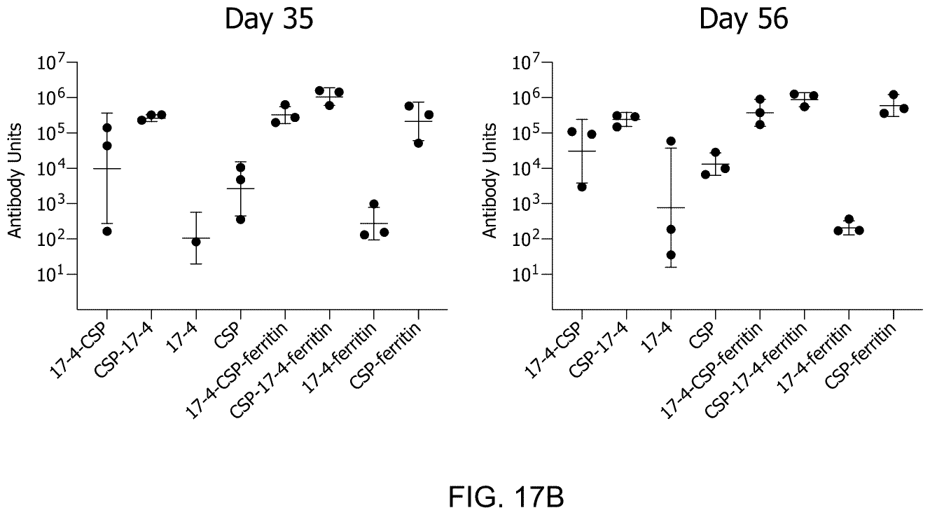

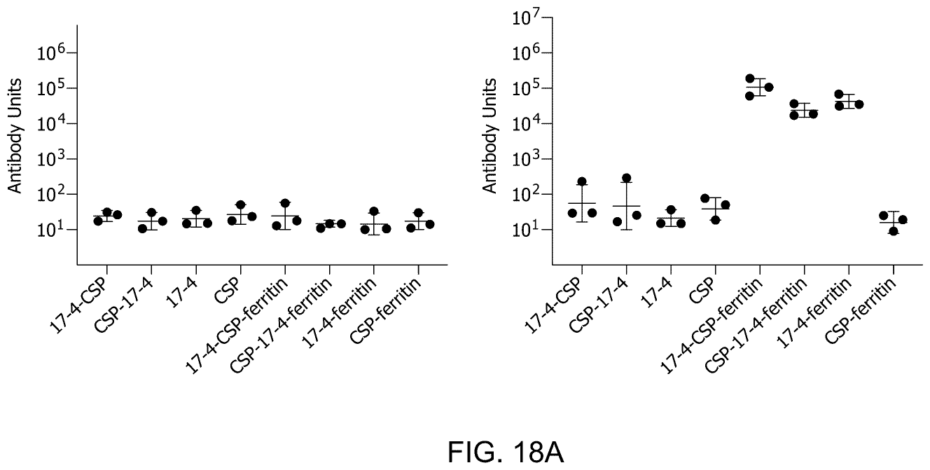

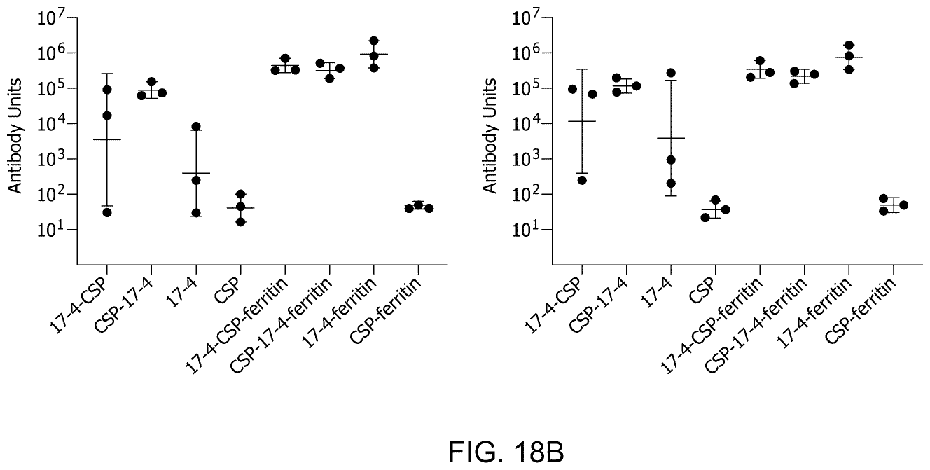

All three lead immunogens were more thermostable than WT D3 (FIG. 1F and FIG. 3). Melting temperatures (Tₘ) were measured by differential scanning fluorimetry (DSF) to determine if the enhancing mutations stabilize the designed antigens and fully compensate for the removal of the stabilizing glycan. Glycosylated WT D3 had a Tₘ of 48° C., while the non-glycosylated designed immunogens had Tₘs 8-23° C. higher. Interestingly, the immunogen with the highest Tₘ (immunogen 15) was not the same as the immunogen with the highest yield (immunogen 17), suggesting these characteristics are not strictly correlated.

Biolayer interferometry (BLI) was used to measure the integrity of the neutralizing epitope in a quantitative fashion (FIGS. 4A-4D and Table 1). The neutralizing antibody TB31F bound all immunogens with sub-nanomolar affinity that was better than WT D3. These affinities suggest that the neutralizing epitope is unperturbed in all three lead immunogens, and that glycosylation of the WT protein may interfere slightly with the binding of neutralizing antibodies. Similarly, glycosylation of a WT vaccine antigen may mask neutralizing epitopes, supporting the need for a non-glycosylated immunogen.

TABLE 1
The neutralizing mAb TB31F binds lead immunogens
15, 17, and 27 stronger than WT D3. Binding
affinities and kinetic data determined by BLI.

| | K_{D} | Kₐ | K_{dis} | |
|---|---|---|---|---|
| | (nM) | (1/Ms) × 10⁵ | (1/s) × 10⁻⁴ | N |
| WT D3 | 2.5 ± 0.4 | 3.5 ± 0.7 | 8.2 ± 0.9 | 3 |
| Immunogen 15 | 0.8 ± 0.06 | 11 ± 6 | 8.5 ± 0.4 | 3 |
| Immunogen 17 | 0.69 ± 0.09 | 12 ± 6 | 8.0 ± 0.7 | 3 |
| Immunogen 27 | 0.62 ± 0.05 | 13 ± 6 | 7.9 ± 0.4 | 3 |

### Immunogens Elicit Functional Antibodies in Rodents

Rats were immunized with the three lead immunogens and the control antigen WT D3 to determine if the immunogens could elicit an improved immune response (FIG. 5A). Rats were immunized with three doses of 40 μg antigen adjuvanted in Freund's adjuvant (CFA/IFA). All four antigens elicited high titers of IgG that recognized WT D3 (FIG. 5B). The immunogens elicited slightly lower titers than WT D3 consistent with the loss of non-neutralizing epitopes outside of the TB31F epitope.

It was hypothesized that the immunogens would elicit higher levels of functional antibodies, despite the lower overall antibody titers, because the immune response would be focused to the neutralizing epitope. Indeed, pooled serum from rats immunized with immunogens 15 and 17 contained higher transmission-reducing activity (TRA) than WT D3 in a standard membrane feeding assay (SMFA) (FIG. 6). Immunogen 27 elicited no detectable TRA, potentially due to its lower thermostability. The serum from individual animals were further analyzed and found that immunogen 15 elicited moderate TRA in three of the four animals and immunogen 17 elicited moderate TRA in one animal (FIG. 5C). WT D3, however, elicited very little TRA in all four animals, suggesting that the immunogens improved the functional antibody response in rats.

### Optimization of Immunogens Through Reversion to WT Sequence

The partial TRA conferred by immunogens 15 and 17 provided a starting point for further optimization of these immunogens (FIG. 7). mAb 85RF45.1 (and the humanized version TB31F) is the only antibody for which structural information exists, meaning that a comprehensive epitope map of pfs48/45 D3 is lacking. Therefore, mutations in immunogens 15 and 17 may perturb undiscovered neutralizing epitopes, limiting the efficacy of these vaccine antigens. To restore potential neutralizing epitopes, amino acids were iteratively reverted to their WT identity without restoring the N-glycosylation site.

A computational strategy for amino acid reversion that prioritized changes based on predicted thermodynamic stability in ROSETTA was used. Those amino acid reversions that had beneficial or neutral energetic effects were made first and additional designs were created with increasingly energetically unfavorable reversions added. For example, 17-6 has more amino acid reversions and is more homologous to WT D3 than 17-2, but 17-6 is predicted to be less energetically stable than 17-2.

The reversion candidates for immunogens 15 and 17 were expressed in mammalian cell culture and identified several that had purification yields as good or better than the parent immunogen (FIG. 7B). Most of these candidates could be purified as well-behaved monomeric proteins with 15-2 and 17-4 having the highest yields (FIGS. 7D & 7C). The reversion candidates were down-selected based on Tₘ, resulting in two optimized leads: immunogens 15-2 and 17-4 (FIG. 7B and FIG. 7E & 7F). Immunogens 15-2 and 17-4 have yields and thermostabilities at least as good as their parent antigens, which were already improved over WT D3. Furthermore, immunogens 15-2 and 17-4 have 83% and 95% identity to WT D3, compared to the 76% and 79% identities of their respective parent antigens. Thus, immunogens 15-2 and 17-4 retain the benefits of the parent antigens over WT D3 and they may have undiscovered neutralizing epitopes restored.

### Enhancing Immunogenicity of the Antigens Through Nanoparticle Display

Nanoparticle display was used to further improve the efficacy of the immunogens (FIG. 7A). Presentation of an antigen on a protein nanoparticle can dramatically increase functional antibody titers, especially when the antigen is small and poorly immunogenic, like pfs48/45 D3. While the immunogen is likely compatible with many nanoparticle platforms, a genetic fusion to H. pylori ferritin was used to create a single-component self-assembling particle that can be easily manufactured and has an established record of safety in human clinical trials (Houser, K. V. et al. Safety and immunogenicity of a ferritin nanoparticle H2 influenza vaccine in healthy adults: a phase 1 trial. Nat Med 28, 383-391, doi: 10.1038/s41591-021-01660-8 (2022).

Immunogens 15-2 and 17-4 could be clearly expressed as stable intact ferritin fusions. In contrast, WT D3-ferritin exhibited no visible expression. A single purification step using size-exclusion chromatography yielded pure fusion protein that eluted at a size consistent with the formation of a 24-copy nanoparticle (FIGS. 3C & 3D 7G & 7H). Negative-stain electron microscopy images revealed pure nanoparticles and the 2D class averages showed visible antigen displayed on the exterior (FIG. 7I). In summary, antigen optimization resulted in 24-copy single-component nanoparticles displaying pfs48/45 D3 immunogens 83% and 95% identical to the WT sequence (FIG. 8).

Optimized Immunogen Nanoparticles Elicit Significantly Higher TRA than WT D3

Rats were immunized with the optimized immunogen nanoparticles to determine if they elicited functional antibodies (FIG. 9). To better scrutinize their suitability for clinical development, the animals were administered two low doses of antigen (1 μg) adjuvanted in AddaS03, a mimic of the AS03 adjuvant approved for human use (FIG. 9A). Serum was analyzed 14 days after the second immunization, and it was found that both particles elicited high titers of IgG that recognized WT D3 (FIG. 9B). However, the titers elicited by 17-4-ferritin were higher than for 15-2-ferritin. Pooled serum from the group immunized with 17-4-ferritin also exhibited high levels of TRA, while 15-2-ferritin elicited no TRA (FIG. 10). Serum from the individual animals immunized with 17-4-ferritin was further analyzed to find that serum from 4/6 animals had very high TRA >90%, indicating a very potent functional immune response. The two animals in the 17-4-ferritin group that had low TRA also had the lowest antibody titers, suggesting that increasing titers through simple steps like higher dosing or more immunizations may lead to a complete response. In summary, the TRA elicited by two low doses of 17-4-ferritin in a human-grade adjuvant is significantly higher than that elicited by three high doses of WT D3 adjuvanted in CFA/IFA (FIG. 4C).

### Example 4. Mouse Immunization and Challenge to Assess CSP Response

To test the ability of immunogens of the disclosure to protect against infection by Plasmodium in vivo, mice were immunized and then challenged with Pf sporozoites. Briefly, five- to six-week-old CJ57B16 mice (Charles River Laboratories) were immunized with 2.5 μg of antigen on day 0, day 21, and day 42. Each group contained ten mice. Antigens were formulated as a 1:1 ratio in AddaS03™ Adjuvant (InvivoGen) with 100 μl of formulated antigen delivered by subcutaneous injection. Blood was collected on day 21, day 35, and day 56 for analysis. Serum was separated and stored at −80° C.

To determine the protection against sporozoite infection, immunized mice were challenged intradermally at day 64 with 1,000 PbPfCSP sporozoites in 0.1 ml of dissecting medium with 50 ul at two injection sites. The salivary glands of A. stephensi mosquitoes were dissected and gently placed in dissection medium E-199 without l-glutamine containing 0.2% Bovine Serum Albumin. Salivary gland suspension was passed 25 times through 1-ml syringe with a 26-gauge needle to release the SPZ, and then dissecting medium was added to achieve a 0.5 ml final volume. Sporozoites were maintained on ice throughout the process and counted using hemocytometer. Mice were monitored for blood-stage parasitemia by Giemsa-stained thin blood smears from day 4-day 12 post challenge. Sterile protection was defined as mice without detectable parasitemia in the blood during the 14-day follow-up.

The results are shown in FIGS. 15 &16. FIG. 15 shows that mice immunized with either the 17-4-CSp fusion protein or the CSP-17-4 fusion protein showed a wide range of antibody titers. Mice immunized with the fusion proteins attached to ferritin nanoparticles all showed a high titer of anti-CSP antibody. FIG. 16 shows that mice immunized with the CSP-17-4 were protected against lethal challenge with Pf sporozoites.

### Example 5. Elicitation of Anti-Pfs48/45 Antibody in Rabbits

To determine the immunogenicity of the 17-4/CSP fusion proteins, and nanoparticles displaying such fusions, anti-Pfs48/45 antibodies were measured in immunized rabbits. Briefly, approximately eleven-week-old female rabbits (Charles River Laboratories) were immunized with 10 μg of antigen on day 0, day 21, and day 42. Each group contained three rabbits. Antigens were formulated as a 1:1 ratio in AddaS03™ Adjuvant (InvivoGen) with 500 μl of formulated antigen delivered by subcutaneous injection. Blood was collected on day 0, day 21, day 35, and day 56 for analysis. The results are shown in FIGS. 17A & 17.

### DISCUSSION

The examples demonstrate the creation an effective and potent stand-alone Pfs48/45 immunogen through structure-based design. Rats immunized with a low dose of antigen formulated in a clinically relevant adjuvant produce high titers of transmission reducing antibodies. The success of this vaccine, and the SPEEDesign process, can be attributed to an increase in the transmission-blocking quality of the antibody response rather than a simple increase in anti-Pfs48/45 antibody titers (FIG. 11). For example, immunization with WT D3 produces very high antibody titers, but no TRA (FIG. 2). Immunogen 15 produces slightly lower titers than WT D3, but higher TRA, suggesting the quality of the antibody response is improved. Similarly, 17-4-ferritin elicits higher TRA than the parent immunogen 17, but with lower antibody titers. The quality of the antibody response can be quantified by expressing TRA normalized to the level of Pfs48/45 antibodies (i.e., calculating the ratio of % TRA to the titer of Pfs48/45 antibodies measured by ELISA), which clearly indicates that the quality of the antibody response improves through the design process (FIG. 11).

There are several reasons that the design process might improve the quality of the antibody response. First, removal of the large non-native glycan could expose neutralizing epitopes, increasing the antibody response to these epitopes in immunized animals. This conclusion is supported by the fact that the neutralizing mAb TB31F binds more tightly to the immunogens than WT D3. Second, the increased thermostability of the immunogens may increase their duration and conformational stability in the body of immunized animals, particularly in germinal centers where affinity maturation can continue for weeks. Third, redesign of the exposed D2/D3 interaction surface on D3 may reduce immunogenicity to non-natural surfaces created upon extraction of D3 from the full length Pfs48/45. Fourth, the amino acid reversion process may restore undiscovered neutralizing epitopes. Fifth, nanoparticle display may selectively present neutralizing epitopes and/or affect the B cell maturation process in a way that increases neutralizing antibody activity. These factors are not mutually exclusive and there are likely several factors that contribute to the improved antibody response elicited by the designed immunogens.

The immunogens presented here enable a wide array of vaccination strategies that were previously impossible. The creation of a Pfs48/45 antigen without the NxS/T glycosylation motif allows production of the antigen in any organism without the risk of a non-native glycan obscuring functional epitopes. This means the antigen can be faithfully produced in human cells, enabling the use of viral vectored and nucleic acid vaccine platforms. Widely used recombinant platforms like yeast, insect cells, and CHO cells can also be used to produce these antigens without the addition of large branched glycans completely unlike those found in the malaria parasite. These recombinant platforms are particularly enticing because they are well-suited for producing proteins with complex disulfide patterns like Pfs48/45. Furthermore, these recombinant platforms allow co-expression or fusion of many different proteins such as the nanoparticle carrier showcased here.

While the disclosure herein was limited to the analysis of TRA against a homologous parasite strain the Pfs48/45 immunogen may be used to elicit strain-transcending blocking activity. In the Pf3k database of >2,500 P. falciparum samples, there are only 9 polymorphic positions within Pfs48/45 D3, and only three rare polymorphisms (<0.1% frequency) lie within the 85RF45.1/TB31F epitope. Two of these polymorphisms do not perturb 85RF45.1 binding, and while the third has not been tested, there are likely to be conserved neutralizing epitopes on the protein. The immunogens retain the 85RF45.1/TB31F epitope and surrounding residues, suggesting they should elicit strain-transcending antibodies.

The design process presented here is generalizable and may be used to create enhanced non-glycosylated antigens for various pathogens. Many different pathogens have glycosylation patterns that are not recapitulated by recombinant production platforms and that may result in occluded neutralizing epitopes. Simple mutation of the NxS/T motif often destabilizes an antigen, as shown for Pfs48/45, but SPEEDesign can counteract this destabilization to create an effective antigen suitable for recombinant production. The disclosed design method is especially applicable to antigens from parasites that have unique glycosylation pathways, such as Plasmodium, Toxoplasma, Leishmania, and Trypanosoma. RTS,S is the only parasite vaccine approved for human use, but SPEEDesign will facilitate the production of many new parasite antigens and vaccine candidates where the prerequisite information is available. Vaccines against viruses could also benefit from SPEEDesign stabilization of de-glycosylated antigens. Viruses often use glycans to shield neutralizing epitopes from antibodies, and glycan removal may elicit an antibody response to neutralizing epitopes that would otherwise be occluded. For example, arenaviruses with fewer N-linked glycosylation sites elicit higher titers of neutralizing antibodies, suggesting that additional removal of glycans through SPEEDesign will produce even higher neutralizing antibody titers.

Non-glycosylated proteins also have manufacturing and safety benefits over glycoproteins. The precise glycan structure and composition on a glycoprotein depends on production platform and can be influenced by subtle changes in growth conditions. This can lead to heterogeneity within a batch of glycoprotein and variation between batches. Alternatively, the composition of a non-glycosylated antigen is much more homogenous and reproducible. Additionally, glycoproteins can cause unwanted immune reactions if they contain non-human glycans, as was observed in the severe hypersensitivity reaction to a murine glycan on cetuximab. Non-glycosylated proteins would not be at risk for this problem, making SPEEDesign potentially applicable to therapeutic proteins, as well as vaccine antigens, as demonstrated by the development of a potent transmission blocking vaccine for malaria.

1. A modified 6C domain from a 48/45 protein (P48/45) of a Plasmodium spp., wherein the amino acid sequence of the modified 6C domain has at least about 70% homology over the entire length of the sequence of a wild-type (wt) P48/45 6C domain, wherein the amino acid sequence of the modified 6C domain comprises one or more mutations, relative to the wt 6C domain amino acid sequence, that increase the stability of the modified 6C domain relative to the stability of the wt 6C domain, wherein the modified 6C domain retains a 85RF45.1 epitope such that mAb 85RF45.1, or a humanized version thereof, binds the modified 6C domain with high affinity, and wherein at least one of the one or more mutations are at an amino acid position selected from the group consisting of an amino acid position corresponding to S11 of SEQ ID NO: 1, an amino acid position corresponding to S15 of SEQ ID NO:1, an amino acid position corresponding to D22 of SEQ ID NO:1, and an amino acid position corresponding to P69 of SEQ ID NO:1.

2. The modified 6C domain of claim 1, wherein the modified 6C domain comprises five or more mutations, wherein at least five of the mutations are at amino acid positions selected from the group consisting of an amino acid position corresponding to S11 of SEQ ID NO:1, an amino acid position corresponding to S15 of SEQ ID NO:1, an amino acid position corresponding to D22 of SEQ ID NO:1, an amino acid position corresponding to P44 of SEQ ID NO:1, an amino acid position corresponding to L52 of SEQ ID NO: 1, an amino acid position corresponding to P69 of SEQ ID NO:1, and an amino acid position corresponding to G107 of SEQ ID NO:1.

3. The modified 6C domain of claim 1, wherein the modified 6C domain comprises seven or more mutations, wherein at least seven of the mutations are at amino acid positions selected from the group consisting of an amino acid position corresponding to S11 of SEQ ID NO:1, an amino acid position corresponding to S15 of SEQ ID NO:1, an amino acid position corresponding to D22 of SEQ ID NO:1, an amino acid position corresponding to P44 of SEQ ID NO:1, an amino acid position corresponding to L52 of SEQ ID NO: 1, an amino acid position corresponding to P69 of SEQ ID NO:1, and an amino acid position corresponding to G107 of SEQ ID NO:1.

4. The modified 6C domain of claim 1, wherein the mutations are not at a position comprising a contact residue for mAb 85RF45.1, or a humanized version thereof.

5. The modified 6C domain of claim 4, wherein the contact residues of the 85RF45.1 epitope comprise at least 20, optionally at least 24, or all, amino acid residues selected from the group consisting of:
an aspartic acid at a position corresponding to position 31 of SEQ ID NO:1,
a proline at a position corresponding to position 55 of SEQ ID NO:1,
a glycine at a position corresponding to position 56 of SEQ ID NO:1,
an aspartic acid at a position corresponding to position 57 of SEQ ID NO:1,
a isoleucine at a position corresponding to position 58 of SEQ ID NO:1,
a isoleucine at a position corresponding to position 59 of SEQ ID NO:1,
a proline at a position corresponding to position 60 of SEQ ID NO:1,
an aspartic acid at a position corresponding to position 61 of SEQ ID NO:1,
a phenylalanine at a position corresponding to position 64 of SEQ ID NO:1,
a glutamine at a position corresponding to position 65 of SEQ ID NO:1,
a leucine at a position corresponding to position 74 of SEQ ID NO:1,
a glutamic acid at a position corresponding to position 75 of SEQ ID NO:1,
a proline at a position corresponding to position 76 of SEQ ID NO:1,
a serine at a position corresponding to position 77 of SEQ ID NO:1,
an isoleucine at a position corresponding to position 79 of SEQ ID NO:1,
a tyrosine at apposition corresponding to position 81 of SEQ ID NO: 1,
a glutamic acid at a position corresponding to position 95 of SEQ ID NO:1,
an aspartic acid a position corresponding to position 100 of SEQ ID NO:1,
a lysine at a position corroding to position 102 of SEQ ID NO:1,
a lysine at a position corresponding to position 104 of SEQ ID NO:1,
an isoleucine at a position corresponding to position 121 of SEQ ID NO:1,
a lysine at a position corresponding to position 123 of SEQ ID NO:1,
a lysine at a position corresponding to position 124 of SEQ ID NO:1,
an aspartic acid at a position corresponding to position 125 of SEQ ID NO:1,
a lysine at a position corresponding to position 126 of SEQ ID NO:1, and,
a serine at a position corresponding to position 128 of SEQ ID NO:1.

6. The modified 6C domain of claim 1, wherein the modified 6C domain is derived from a wt 6C domain from a Plasmodium species selected from the group consisting of P. falciparum (Pfs), P. vivax (Pvx), P. malariae (Pml), and P. ovale (Pov).

7. The modified 6C domain of claim 1, wherein the wt 6C domain comprises SEQ ID NO:1.

8. The modified 6C domain of claim 1, wherein the modified 6C domain comprises a Tₘ of at least 60° C.

9. The modified 6C domain of claim 1, wherein the one or more mutations comprise an alanine at a position corresponding to position 11 of SEQ ID NO:1, an alanine at a position corresponding to position 15 of SEQ ID NO:1, a lysine at a position corresponding to position 22 of SEQ ID NO:1, a lysine at a position corresponding to position 44 of SEQ ID NO:1, a methionine at a position corresponding to position 52 of SEQ ID NO:1, a glutamine at a position corresponding to position 69 of SEQ ID NO: 1, and a lysine at a position corresponding to position 107 of SEQ ID NO:1.

10. The modified 6C domain of claim 1, wherein the amino acid sequence of the modified 6C domain has at least about 90% homology to SEQ ID NO:5, and wherein the modified 6C domain comprises an alanine at a position corresponding to position 11 of SEQ ID NO:1, an alanine at a position corresponding to position 15 of SEQ ID NO:1, a lysine at a position corresponding to position 22 of SEQ ID NO:1, a lysine at a position corresponding to position 44 of SEQ ID NO:1, a methionine at a position corresponding to position 52 of SEQ ID NO:1, a glutamine at a position corresponding to position 69 of SEQ ID NO:1, and a lysine at a position corresponding to position 107 of SEQ ID NO: 1.

11. The modified 6C domain of claim 1, wherein the modified 6C domain comprises a sequence selected from the group consisting of SEQ ID NO:5, SEQ ID NO:4, SEQ ID NO:13, SEQ ID NO14, and SEQ ID NO:15.

12. An isolated protein comprising the modified 6C domain of claim 1.

13. A fusion protein comprising a monomeric, self-assembling subunit protein joined to the modified 6C domain of claims 1-11 or the isolated protein of claim 12.

14. The fusion protein of claim 13, wherein the monomeric, self-assembling subunit protein comprises ferritin, dihydrolipoyl acetyltransferase (E2P), Lumazine Synthase (LuS), hepatitis B surface antigen (HBsAg), or human papilloma virus protein LI (HPV LI).

15. A nucleic acid molecule comprising a nucleotide sequence encoding the modified 6C domain of claim 1.

16. A nanoparticle comprising the modified 6C domain of claim 1.

17. A method of eliciting an immune response against Plasmodium falciparum in an individual, the method comprising administering to the individual a modified 6C domain of claim 1.
